(19) United States
(12) Patent Application Publication
(10) Pub. No.: US 2026/0325556 A1
(43) Pub. Date: Oct. 22, 2026

(54) ORAL TABLET FORMULATIONS

(71) Applicant: Gilead Sciences, Inc., Foster City, CA (US)

(72) Inventors: James B. C. Mack, San Mateo, CA (US); Isabel A. Olson, Livermore, CA (US); Murali Subramanian, Foster City, CA (US); Zaikuan Yu, Foster City, CA (US)

(21) Appl. No.: 19/544,666

(22) Filed: Feb. 19, 2026

Related U.S. Application Data
(60) Provisional application No. 63/761,619, filed on Feb. 21, 2025.

(51) Int. Cl. A61K 31/675; A61K 9/20; A61K 9/28; A61K 31/4725

(52) U.S. Cl. CPC A61K 31/675; A61K 9/2013; A61K 9/2018; A61K 9/2027; A61K 9/2031; A61K 9/2054; A61K 9/28; A61K 31/4725

(57) ABSTRACT

The present disclosure relates to pharmaceutical formulations comprising a HIV capsid inhibitor and methods for the prevention of a human immunodeficiency virus (HIV) infection in a patient.

### CROSS REFERENCE TO RELATED APPLICATIONS

This application claims the benefit of U.S. Provisional Application No. 63/761,619, filed Feb. 21, 2025, the entire contents of which is hereby incorporated by reference in its entirety.

### BACKGROUND

The viral capsid protein (CA) is essential for multiple stages of the HIV life cycle. During viral maturation following the processing of Gag polyprotein by the HIV protease, CA self-assembles into the conical shaped core characteristic of mature HIV-1 virions. Contained within this capsid core are the viral RNA, nucleocapsid, reverse transcriptase, and integrase. Failure to generate a suitable core precludes infectivity. In addition, CA contributes to multiple essential processes during the early stages of HIV replication, including important roles in regulating proper capsid core disassembly (uncoating) kinetics to ensure efficient and productive viral DNA synthesis via coupled reverse transcription, and contributes to the active transport of pre-integration complexes into the nuclear compartment to support viral DNA integration into transcriptionally active loci. Defects in the proper function of capsid ultimately inhibit efficient nuclear uptake and integration of viral DNA into the host genome.

Human immunodeficiency virus type 1 infection is a life-threatening and serious disease of major public health significance, with approximately 38 million people infected worldwide and approximately 26 million on antiretroviral (ARV) treatment (UNAIDS. Global HIV & AIDS statistics, 2020 fact sheet). Advances in combination ARV therapy for HIV have led to significant improvements in morbidity and mortality by suppressing viral replication, preserving immunologic function, and averting disease progression to AIDS.

### SUMMARY

The present disclosure provides, inter alia, a tablet comprising a compound of Formula Ia:

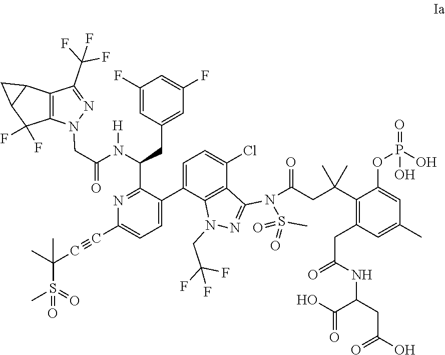 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, and one or more pharmaceutically acceptable excipients.

The present disclosure further provides a method of preventing human immunodeficiency virus (HIV) infection in a patient, comprising administering to the patient a tablet provided herein.

The present disclosure further provides a tablet provided herein, for use in any of the methods described herein.

The present disclosure further provides use of a tablet provided herein, for use in any of the methods described herein.

The present disclosure further provides use of a tablet provided herein, for use in preparation of a medicament for use in any of the methods described herein.

### DESCRIPTION OF DRAWINGS

FIG. 1 shows a manufacturing process flow diagram for preparing the tablets described in Example 1.

FIGS. 2-5 show results of in vivo dog studies described in Example 2.

FIG. 6 shows plasma pharmacokinetic (PK) profiles of encequidar following intravenous (IV) and oral (PO) administration in non-clinical species (monkey, dog, and rat). Plasma concentration-time profiles are shown for IV administration at 1 mg/kg (blue circles) and PO administration at 10 mg/kg (red squares).

FIG. 7 shows pharmacokinetic (PK) profiles of orally administered digoxin (5 mg/kg) and paclitaxel (5 mg/kg) in Sprague-Dawley rats, paclitaxel (5 mg/kg) in beagle dogs, and talinolol (1 mg/kg) in cynomolgus monkeys with and without efflux inhibitors. Encequidar (5 mg/kg, red squares) and elacridar (10 mg/kg, green triangles) were co-administered orally to evaluate their effects on substrate plasma concentrations.

FIG. 8 shows pharmacokinetic (PK) profiles of single-dose intravenous (IV) administration of apixaban (0.33 mg/kg), talinolol (0.33 mg/kg), and lenacapavir (0.33 mg/kg) in beagle dogs, and paclitaxel (1 mg/kg) in Sprague-Dawley rats. Plasma concentration-time curves are shown with) and without co-administration of the P-gp inhibitors (5 mg/kg PO).

FIG. 9 shows improvement in bioavailability of lenacapavir in pentagastrin pretreated dogs upon administration of Compound 1, 350 mg fixed PO and solution vehicle.

### DETAILED DESCRIPTION

Pre-exposure prophylaxis (PrEP) strategies have been used to prevent transmission of HIV-1 infection. In locations where uptake of PrEP is high, there has been a significant population-level reduction in HIV incidence, demonstrating the potential for PrEP to contribute to population-wide HIV control (see e.g., Grulich et al, The Lancet. HIV, 2018; 5(11): e629-e37; and Sullivan et al, Abstract LBPEC036, International AIDS Conference (IAC); July 2018, 23-27). However, for many individuals at risk for HIV, the uptake of daily oral PrEP has been a challenge. In the US, it is estimated that 1.1 million persons are at risk for HIV, yet only 240,000 are on Descovy or Truvada, and another 400,000 more started, but have stopped, daily oral PrEP. Several reasons likely contribute to the suboptimal uptake of PrEP, including lack of health system accessibility, medical mistrust due to experiences of homophobia, transphobia, and stigma, lack of willingness to take daily oral PrEP, concerns about side effects, and low self-perception of risk (see e.g., Center for Disease Control and Prevention, HIV/AIDS: Pre-exposure prophylaxis (PrEP); cdc.gov/hiv/basics/prep.html, 2017; and Wood et al, AIDS Behav. 2019; 23(10): 2719-29).

Lenacapavir (LEN), a human immunodeficiency virus type 1 (HIV-1) capsid inhibitor, has the potential to provide an additional option for populations at risk of HIV acquisition. Lenacapavir can help address substantial existing PrEP barriers including 1) requirement for daily adherence, 2) stigma and concerns about disclosure and discrimination or other social harms, 3) oral medication-associated adverse events (AEs), including gastrointestinal tolerability, and 4) challenges with access to health care providers in overburdened health systems or in geographical PrEP deserts. Thus, LEN has the potential to increase the uptake of, adherence to, and thereby the scalability of PrEP, thus contributing to the overarching goal of ending the HIV-1 epidemic.

The present invention relates to pharmaceutical compositions (e.g., tablets) comprising (2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl)but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetyl)-L-aspartic acid (i.e., Compound 1, structure shown below; see e.g. U.S. Pat. No. 11,787,825, the disclosure of which is incorporated herein by reference in its entirety).

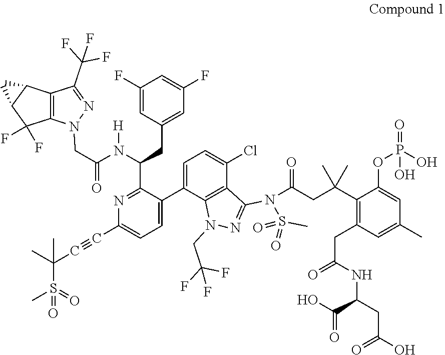 [SMILES: Cc1cc(CC(=O)N[C@@H](CC(=O)O)C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

Compound 1 converts to lenacapavir (i.e., N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide), an HIV capsid inhibitor that is in development as a long-acting treatment for HIV, in the gastrointestinal tract when administered to a subject (e.g., a human patient).

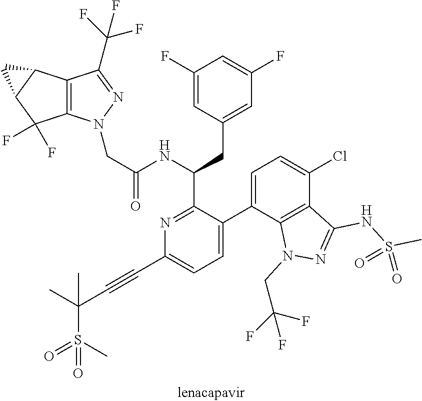 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

Synthesis and characterization of lenacapavir, and salts thereof, are described, for example, in US20180051005 and US20190300505, the contents of each of which are hereby incorporated by reference in their entireties. Various forms and/or uses of the compounds of lenacapavir are disclosed, for example, in US20190083478, US20190084963, U.S. Pat. No. 20200038389A1, and US20210188815, the contents of each of which are hereby incorporated by reference in their entireties.

As used herein and in the appended claims, the singular forms “a” and “an”, and “the” include plural referents unless the context clearly dictates otherwise. Thus, e.g., reference to “the compound” includes a plurality of such compounds, “a tablet” or “the tablet” includes reference to one or more tablets, and reference to “the assay” includes reference to one or more assays, and so forth.

The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds and salts described herein contain one or more asymmetric centers and/or hindered rotation about a bond axis and may thus give rise to enantiomers, diastereomers, and other stereoisomeric forms that may be defined, in terms of absolute stereochemistry, as (R)- or(S)-. The present disclosure is meant to include all such possible isomers, including racemic mixtures, scalemic mixtures, diastereomeric mixtures, optically pure forms and intermediate mixtures. Optically active (R)- and(S)-isomers may be prepared using chiral synthons or chiral reagents, or resolved using conventional techniques.

Except as expressly defined otherwise, the present disclosure includes all tautomers of compounds detailed herein, even if only one tautomer is expressly represented (e.g., both tautomeric forms are intended and described by the presentation of one tautomeric form where a pair of two tautomers may exist). For example, if reference is made to a compound containing an amide (e.g., by structure or chemical name), it is understood that the corresponding imidic acid tautomer is included by this disclosure and described the same as if the amide were expressly recited either alone or together with the imidic acid. Where more than two tautomers may exist, the present disclosure includes all such tautomers even if only a single tautomeric form is depicted by chemical name and/or structure.

Compounds described herein may have chiral centers and/or geometric isomeric centers (E- and Z-isomers), and it is to be understood that all such optical, enantiomeric, diastereoisomeric and geometric isomers are encompassed. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s).

One skilled in the art understands that a compound structure may be named or identified using commonly recognized nomenclature systems and symbols. By way of example, the compound may be named or identified with common names, systematic or non-systematic names. The nomenclature systems and symbols that are commonly recognized in the art of chemistry including but not limited to Chemical Abstract Service (CAS) and International Union of Pure and Applied Chemistry (IUPAC). Accordingly, the compound structure for Compound 1 provided above may also be named or identified as (2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl)but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetyl)-L-aspartic acid.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, the term “about X” includes description of “X”.

As used herein, the term “consists essentially of” means that, in addition to the mandatory ingredients, specific further ingredients can be present, namely those not materially affecting the essential characteristics of the composition or component in question. Compositions or components herein which “consist essentially of” the specified ingredients may consist only of the specified ingredients, with no other ingredients present.

Kolliphor® HS-15 (CAS No. 70142-34-6) is a non-ionic solubilizer and emulsifying agent obtained by reacting 15 moles of ethylene oxide with 1 mole of 12-hydroxystearic acid. Kolliphor® HS-15 consists of polyglycol mono- and di-esters of 12-hydroxystearic acid (=lipophilic part) and of about 30% of free polyethylene glycol (=hydrophilic part). Kolliphor® HS-15 is also known as Macrogol 15 Hydroxystearate (Ph. Eur.), or Polyoxyl 15 Hydrostearate (USP/NF).

Kolliphor® RH 40 is a non-ionic solubilizer and emulsifying agent obtained by reacting 1 mole of hydrogenated castor oil with 40 moles of ethylene oxide. The main constituent of Kolliphor® RH 40 is glycerol polyethylene glycol hydroxy-stearate, which, together with fatty acid glycerol polyglycol esters, forms the hydrophobic part of the product. The hydrophilic part consists of polyethylene glycols and glycerol ethoxylate. Kolliphor® RH 40 is also known as Macroglycerol Hydroxystearate (Ph. Eur.) or Polyoxyl 40 Hydrogenated Castor Oil (USP/NF).

### Pharmaceutical Compositions

Accordingly, the present disclosure provides pharmaceutical compositions comprising a compound of Formula Ia or Ib:

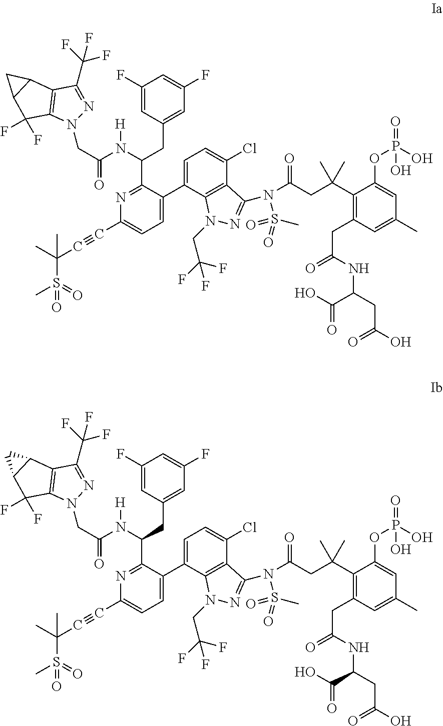 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1 ; Cc1cc(CC(=O)N[C@@H](CC(=O)O)C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, and at least one pharmaceutically acceptable carrier.

Pharmaceutical compositions of the disclosure are formulated so as to allow the active ingredients contained therein to be bioavailable upon administration of the composition to a patient. Compositions that will be administered to a patient take the form of one or more dosage units (e.g., one or more tablets). Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a prophylactically effective amount of the compound of Formula Ia or Ib, or a pharmaceutically acceptable salt thereof, for prevention of an HIV infection, or reducing the risk of acquiring an HIV infection, as described herein.

The term “prophylactically effective amount” or “effective amount” of a compound described herein means an amount sufficient to effect prevention of an HIV infection, or reduce the risk of acquiring an HIV infection, when administered to a subject. The prophylactically effective amount may vary, for example, depending on the subject, the weight and age of the subject, and the manner of administering, which can readily be determined by one of ordinary skill in the art.

As used herein, “pharmaceutically acceptable carrier” is meant to refer to any adjuvant, carrier, excipient, filler, disintegrant, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, the compound of Formula Ia or Ib is administered as a salt, such as a pharmaceutically acceptable salt. A salt generally refers to a derivative of a disclosed compound wherein the parent compound is modified by converting an existing acid or base moiety to its salt form. A pharmaceutically acceptable salt is one that, within the scope of sound medical judgment, is suitable for use in contact with the tissues of human beings and animals without excessive toxicity, irritation, allergic response, or other problem or complication, commensurate with a reasonable benefit/risk ratio. Examples of pharmaceutically acceptable salts include, but are not limited to, mineral or organic acid salts of basic residues such as amines, alkali or organic salts of acidic residues such as carboxylic acids, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

In some embodiments, the pharmaceutical composition provided herein is a tablet. In some embodiments, the tablet provided herein comprises a compound of Formula Ia or Ib, or a pharmaceutically acceptable salt thereof, and one or more pharmaceutically acceptable excipients. In some embodiments, the tablet provided herein comprises a compound of Formula Ia, or a pharmaceutically acceptable salt thereof, and one or more pharmaceutically acceptable excipients. In some embodiments, the tablet provided herein comprises a compound of Formula Ib, or a pharmaceutically acceptable salt thereof, and one or more pharmaceutically acceptable excipients.

In some embodiments, the active ingredient is a compound of Formula Ia, or a pharmaceutically acceptable salt thereof. In some embodiments, the active ingredient is a compound of Formula Ib, or a pharmaceutically acceptable salt thereof.

In some embodiments, the active ingredient is a compound of Formula Ia (i.e., a free acid form of the compound of Formula Ia). In some embodiments, the active ingredient is a compound of Formula Ib (i.e., a free acid form of the compound of Formula Ib).

In some embodiments, the active ingredient is an amorphous form of the free acid form of the compound of Formula Ia. In some embodiments, the active ingredient is an amorphous form of the free acid form of the compound of Formula Ib.

In some embodiments, the tablet disclosed herein comprises a compound of Formula Ia or Ib, or a pharmaceutically acceptable salt thereof, and one or more pharmaceutically acceptable excipients selected from the group consisting of a filler, a disintegrant, and a lubricant.

In some embodiments, the tablet disclosed herein comprises a compound of Formula Ia or Ib, or a pharmaceutically acceptable salt thereof, a filler, a disintegrant, and a lubricant.

In some embodiments, the tablet disclosed herein comprises a compound of Formula Ia or Ib, or a pharmaceutically acceptable salt thereof, about 20 w/w % to about 55 w/w % of one or more fillers, about 1 w/w % to 15 w/w % of one or more disintegrants, and about 1 w/w % to 5 w/w % of one or more lubricants.

In some embodiments, the tablet disclosed herein comprises a compound of Formula Ia or Ib, or a pharmaceutically acceptable salt thereof, about 20 w/w % to about 55 w/w % of the one or more fillers, about 4 w/w % to about 15 w/w % of the one or more disintegrants, and about 1 w/w % to about 5 w/w % of the one or more lubricant.

In some embodiments, the one or more fillers are each independently selected from microcrystalline cellulose, mannitol, lactose monohydrate, and silicified microcrystalline cellulose. In some embodiments, the one or more fillers are each independently selected from microcrystalline cellulose, mannitol, and silicified microcrystalline cellulose. In some embodiments, the one or more fillers are each independently selected from microcrystalline cellulose and mannitol. In some embodiments, the disintegrant is crospovidone. In some embodiments, the lubricant is magnesium stearate.

In some embodiments, the tablet provided herein comprises about 30 w/w % to about 80 w/w % of the compound of Formula Ia or Ib, or a pharmaceutically acceptable salt thereof, for example, about 30 w/w %, about 35 w/w %, about 40 w/w %, about 45 w/w %, about 50 w/w %, about 55 w/w %, about 60 w/w %, about 65 w/w %, about 70 w/w %, about 75 w/w %, or about 80 w/w % of the compound of Formula Ia or Ib, or a pharmaceutically acceptable salt thereof.

In some embodiments, the tablet provided herein comprises about 30 w/w % to about 80 w/w % of a free acid form of the compound of Formula Ia or Ib. In some embodiments, the tablet provided herein comprises about 30 w/w % to about 80 w/w % of a free acid form of the compound of Formula Ia. In some embodiments, the tablet provided herein comprises about 30 w/w % to about 80 w/w % of a free acid form of the compound of Formula Ib. In some embodiments, the tablet provided herein comprises about 30 w/w % to about 80 w/w % of an amorphous form of the compound of Formula Ia. In some embodiments, the tablet provided herein comprises about 30 w/w % to about 80 w/w % of an amorphous form of the compound of Formula Ib.

In some embodiments, the tablet provided herein comprises about 35 w/w % to about 75 w/w % of a free acid form of the compound of Formula Ia or Ib. In some embodiments, the tablet provided herein comprises about 35 w/w % to about 75 w/w % of a free acid form of the compound of Formula Ia. In some embodiments, the tablet provided herein comprises about 35 w/w % to about 75 w/w % of a free acid form of the compound of Formula Ib. In some embodiments, the tablet provided herein comprises about 35 w/w % to about 75 w/w % of an amorphous form of the compound of Formula Ia. In some embodiments, the tablet provided herein comprises about 35 w/w % to about 75 w/w % of an amorphous form of the compound of Formula Ib.

In some embodiments, the tablet provided herein comprises about 40 w/w % to about 70 w/w % of a free acid form of the compound of Formula Ia or Ib. In some embodiments, the tablet provided herein comprises about 40 w/w % to about 70 w/w % of a free acid form of the compound of Formula Ia. In some embodiments, the tablet provided herein comprises about 40 w/w % to about 70 w/w % of a free acid form of the compound of Formula Ib. In some embodiments, the tablet provided herein comprises about 40 w/w % to about 70 w/w % of an amorphous form of the compound of Formula Ia. In some embodiments, the tablet provided herein comprises about 40 w/w % to about 70 w/w % of an amorphous form of the compound of Formula Ib.

In some embodiments, the tablet provided herein comprises about 50 w/w % to about 70 w/w % of a free acid form of the compound of Formula Ia or Ib. In some embodiments, the tablet provided herein comprises about 50 w/w % to about 70 w/w % of a free acid form of the compound of Formula Ia. In some embodiments, the tablet provided herein comprises about 50 w/w % to about 70 w/w % of a free acid form of the compound of Formula Ib. In some embodiments, the tablet provided herein comprises about 50 w/w % to about 70 w/w % of an amorphous form of the compound of Formula Ia. In some embodiments, the tablet provided herein comprises about 50 w/w % to about 70 w/w % of an amorphous form of the compound of Formula Ib.

In some embodiments, the tablet provided herein comprises about 55 w/w % to about 65 w/w % of a free acid form of the compound of Formula Ia or Ib. In some embodiments, the tablet provided herein comprises about 55 w/w % to about 65 w/w % of a free acid form of the compound of Formula Ia. In some embodiments, the tablet provided herein comprises about 55 w/w % to about 65 w/w % of a free acid form of the compound of Formula Ib. In some embodiments, the tablet provided herein comprises about 55 w/w % to about 65 w/w % of an amorphous form of the compound of Formula Ia. In some embodiments, the tablet provided herein comprises about 55 w/w % to about 65 w/w % of an amorphous form of the compound of Formula Ib.

In some embodiments, the tablet provided herein comprises about 58 w/w % to about 62 w/w % of a free acid form of the compound of Formula Ia or Ib. In some embodiments, the tablet provided herein comprises about 58 w/w % to about 62 w/w % of a free acid form of the compound of Formula Ia. In some embodiments, the tablet provided herein comprises about 58 w/w % to about 62 w/w % of a free acid form of the compound of Formula Ib. In some embodiments, the tablet provided herein comprises about 58 w/w % to about 62 w/w % of an amorphous form of the compound of Formula Ia. In some embodiments, the tablet provided herein comprises about 58 w/w % to about 62 w/w % of an amorphous form of the compound of Formula Ib.

In some embodiments, the tablet provided herein comprises about 59 w/w % to about 61 w/w % of a free acid form of the compound of Formula Ia or Ib. In some embodiments, the tablet provided herein comprises about 59 w/w % to about 61 w/w % of a free acid form of the compound of Formula Ia. In some embodiments, the tablet provided herein comprises about 59 w/w % to about 61 w/w % of a free acid form of the compound of Formula Ib. In some embodiments, the tablet provided herein comprises about 59 w/w % to about 61 w/w % of an amorphous form of the compound of Formula Ia. In some embodiments, the tablet provided herein comprises about 59 w/w % to about 61 w/w % of an amorphous form of the compound of Formula Ib.

In some embodiments, the tablet provided herein comprises about 60 w/w % of a free acid form of the compound of Formula Ia or Ib. In some embodiments, the tablet provided herein comprises about 60 w/w % of a free acid form of the compound of Formula Ia. In some embodiments, the tablet provided herein comprises about 60 w/w % of a free acid form of the compound of Formula Ib. In some embodiments, the tablet provided herein comprises about 60 w/w % of an amorphous form of the compound of Formula Ia. In some embodiments, the tablet provided herein comprises about 60 w/w % of an amorphous form of the compound of Formula Ib.

In some embodiments, the tablet provided herein comprises about 40 w/w % of a free acid form of the compound of Formula Ia or Ib. In some embodiments, the tablet provided herein comprises about 40 w/w % of a free acid form of the compound of Formula Ia. In some embodiments, the tablet provided herein comprises about 40 w/w % of a free acid form of the compound of Formula Ib. In some embodiments, the tablet provided herein comprises about 40 w/w % of an amorphous form of the compound of Formula Ia. In some embodiments, the tablet provided herein comprises about 40 w/w % of an amorphous form of the compound of Formula Ib.

In some embodiments, the tablet provided herein comprises about 70 w/w % of a free acid form of the compound of Formula Ia or Ib. In some embodiments, the tablet provided herein comprises about 70 w/w % of a free acid form of the compound of Formula Ia. In some embodiments, the tablet provided herein comprises about 70 w/w % of a free acid form of the compound of Formula Ib. In some embodiments, the tablet provided herein comprises about 70 w/w % of an amorphous form of the compound of Formula Ia. In some embodiments, the tablet provided herein comprises about 70 w/w % of an amorphous form of the compound of Formula Ib.

In some embodiments, the tablet comprises the compound of Formula Ia or Ib, or a pharmaceutically acceptable salt thereof, and one or more pharmaceutically acceptable excipients selected from mannitol, microcrystalline cellulose, crospovidone, poloxamer 407, and magnesium stearate, or any combination thereof. In some embodiments, the tablet comprises the free acid form of the compound of Formula Ia or Ib, and one or more pharmaceutically acceptable excipients selected from mannitol, microcrystalline cellulose, crospovidone, poloxamer 407, and magnesium stearate, or any combination thereof. In some embodiments, the tablet comprises the free acid form of the compound of Formula Ia, and one or more pharmaceutically acceptable excipients selected from mannitol, microcrystalline cellulose, crospovidone, poloxamer 407, and magnesium stearate, or any combination thereof. In some embodiments, the tablet comprises the free acid form of the compound of Formula Ib, and one or more pharmaceutically acceptable excipients selected from mannitol, microcrystalline cellulose, crospovidone, poloxamer 407, and magnesium stearate, or any combination thereof. In some embodiments, the tablet comprises an amorphous form of the free acid form of the compound of Formula Ia, and one or more pharmaceutically acceptable excipients selected from mannitol, microcrystalline cellulose, crospovidone, poloxamer 407, and magnesium stearate, or any combination thereof. In some embodiments, the tablet comprises an amorphous form of the free acid form of the compound of Formula Ib, and one or more pharmaceutically acceptable excipients selected from mannitol, microcrystalline cellulose, crospovidone, poloxamer 407, and magnesium stearate, or any combination thereof.

In some embodiments, the tablet comprises the compound of Formula Ia or Ib, or a pharmaceutically acceptable salt thereof, and one or more pharmaceutically acceptable excipients selected from mannitol, microcrystalline cellulose, crospovidone, and magnesium stearate, or any combination thereof. In some embodiments, the tablet comprises the free acid form of the compound of Formula Ia or Ib, and one or more pharmaceutically acceptable excipients selected from mannitol, microcrystalline cellulose, crospovidone, and magnesium stearate, or any combination thereof. In some embodiments, the tablet comprises the free acid form of the compound of Formula Ia, and one or more pharmaceutically acceptable excipients selected from mannitol, microcrystalline cellulose, crospovidone, and magnesium stearate, or any combination thereof. In some embodiments, the tablet comprises the free acid form of the compound of Formula Ib, and one or more pharmaceutically acceptable excipients selected from mannitol, microcrystalline cellulose, crospovidone, and magnesium stearate, or any combination thereof. In some embodiments, the tablet comprises an amorphous form of the free acid form of the compound of Formula Ia, and one or more pharmaceutically acceptable excipients selected from mannitol, microcrystalline cellulose, crospovidone, and magnesium stearate, or any combination thereof. In some embodiments, the tablet comprises an amorphous form of the free acid form of the compound of Formula Ib, and one or more pharmaceutically acceptable excipients selected from mannitol, microcrystalline cellulose, crospovidone, and magnesium stearate, or any combination thereof.

In some embodiments, the tablet comprises the compound of Formula Ia or Ib, or a pharmaceutically acceptable salt thereof, and one or more pharmaceutically acceptable excipients selected from microcrystalline cellulose, crospovidone, and magnesium stearate, or any combination thereof. In some embodiments, the tablet comprises the free acid form of the compound of Formula Ia or Ib, and one or more pharmaceutically acceptable excipients selected from microcrystalline cellulose, crospovidone, and magnesium stearate, or any combination thereof. In some embodiments, the tablet comprises the free acid form of the compound of Formula Ia, and one or more pharmaceutically acceptable excipients selected from microcrystalline cellulose, crospovidone, and magnesium stearate, or any combination thereof. In some embodiments, the tablet comprises the free acid form of the compound of Formula Ib, and one or more pharmaceutically acceptable excipients selected from microcrystalline cellulose, crospovidone, and magnesium stearate, or any combination thereof. In some embodiments, the tablet comprises an amorphous form of the free acid form of the compound of Formula Ia, and one or more pharmaceutically acceptable excipients selected from microcrystalline cellulose, crospovidone, and magnesium stearate, or any combination thereof.

In some embodiments, the tablet comprises an amorphous form of the free acid form of the compound of Formula Ib, and one or more pharmaceutically acceptable excipients selected from microcrystalline cellulose, crospovidone, and magnesium stearate, or any combination thereof.

In some embodiments, the tablet comprises the compound of Formula Ia or Ib, or a pharmaceutically acceptable salt thereof, mannitol, microcrystalline cellulose, crospovidone, poloxamer 407, and magnesium stearate. In some embodiments, the tablet comprises the free acid form of the compound of Formula Ia or Ib, mannitol, microcrystalline cellulose, crospovidone, poloxamer 407, and magnesium stearate. In some embodiments, the tablet comprises the free acid form of the compound of Formula Ia, mannitol, microcrystalline cellulose, crospovidone, poloxamer 407, and magnesium stearate. In some embodiments, the tablet comprises the free acid form of the compound of Formula Ib, mannitol, microcrystalline cellulose, crospovidone, poloxamer 407, and magnesium stearate. In some embodiments, the tablet comprises an amorphous form of the free acid form of the compound of Formula Ia, mannitol, microcrystalline cellulose, crospovidone, poloxamer 407, and magnesium stearate. In some embodiments, the tablet comprises an amorphous form of the free acid form of the compound of Formula Ib, mannitol, microcrystalline cellulose, crospovidone, poloxamer 407, and magnesium stearate.

In some embodiments, the tablet comprises the compound of Formula Ia or Ib, or a pharmaceutically acceptable salt thereof, mannitol, microcrystalline cellulose, crospovidone, and magnesium stearate. In some embodiments, the tablet comprises the free acid form of the compound of Formula Ia or Ib, mannitol, microcrystalline cellulose, crospovidone, and magnesium stearate. In some embodiments, the tablet comprises the free acid form of the compound of Formula Ia, mannitol, microcrystalline cellulose, crospovidone, and magnesium stearate. In some embodiments, the tablet comprises the free acid form of the compound of Formula Ib, mannitol, microcrystalline cellulose, crospovidone, and magnesium stearate. In some embodiments, the tablet comprises an amorphous form of the free acid form of the compound of Formula Ia, mannitol, microcrystalline cellulose, crospovidone, and magnesium stearate. In some embodiments, the tablet comprises an amorphous form of the free acid form of the compound of Formula Ib, mannitol, microcrystalline cellulose, crospovidone, and magnesium stearate.

In some embodiments, the tablet comprises the compound of Formula Ia or Ib, or a pharmaceutically acceptable salt thereof, microcrystalline cellulose, crospovidone, and magnesium stearate. In some embodiments, the tablet comprises the free acid form of the compound of Formula Ia or Ib, microcrystalline cellulose, crospovidone, and magnesium stearate. In some embodiments, the tablet comprises the free acid form of the compound of Formula Ia, microcrystalline cellulose, crospovidone, and magnesium stearate. In some embodiments, the tablet comprises the free acid form of the compound of Formula Ib, microcrystalline cellulose, crospovidone, and magnesium stearate. In some embodiments, the tablet comprises an amorphous form of the free acid form of the compound of Formula Ia, microcrystalline cellulose, crospovidone, and magnesium stearate. In some embodiments, the tablet comprises an amorphous form of the free acid form of the compound of Formula Ib, microcrystalline cellulose, crospovidone, and magnesium stearate.

In some embodiments, the tablet comprises about 10 w/w % to about 30 w/w % of mannitol, for example, about 10 w/w %, about 11 w/w %, about 12 w/w %, about 13 w/w %, about 14 w/w %, about 15 w/w %, about 16 w/w %, about 17 w/w %, about 18 w/w %, about 19 w/w %, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, about 30 w/w % mannitol.

In some embodiments, the tablet comprises about 12 w/w % to about 30 w/w % of mannitol. In some embodiments, the tablet comprises about 10 w/w % to about 20 w/w % of mannitol. In some embodiments, the tablet comprises about 10 w/w % to about 17 w/w % of mannitol. In some embodiments, the tablet comprises about 12 w/w % to about 17 w/w % of mannitol. In some embodiments, the tablet comprises about 11 w/w % mannitol. In some embodiments, the tablet comprises about 11.75 w/w % mannitol. In some embodiments, the tablet comprises about 12 w/w % mannitol. In some embodiments, the tablet comprises about 12.75 w/w % mannitol. In some embodiments, the tablet comprises about 14 w/w % mannitol. In some embodiments, the tablet comprises about 14.25 w/w % mannitol. In some embodiments, the tablet comprises about 16 w/w % mannitol. In some embodiments, the tablet comprises about 16.75 w/w % mannitol. In some embodiments, the tablet comprises about 20 w/w % to about 30 w/w % of mannitol. In some embodiments, the tablet comprises about 25 w/w % to about 30 w/w % of mannitol. In some embodiments, the tablet comprises about 25 w/w % to about 28 w/w % of mannitol. In some embodiments, the tablet comprises about 26 w/w % of mannitol. In some embodiments, the tablet comprises about 26.75 w/w % of mannitol. In some embodiments, the tablet comprises about 27 w/w % of mannitol.

In some embodiments, the tablet comprises about 0.1 w/w % to about 5 w/w % poloxamer 407, for example, about 0.1 w/w %, about 0.5 w/w %, about 1 w/w %, about 2 w/w %, about 3 w/w %, about 4 w/w %, or about 5 w/w % poloxamer 407. In some embodiments, the tablet comprises about 1 w/w % to about 5 w/w % poloxamer 407. In some embodiments, the tablet comprises about 2 w/w % to about 4 w/w % poloxamer 407. In some embodiments, the tablet comprises about 3 w/w % poloxamer 407.

In some embodiments, the tablet comprises about 10 w/w % to about 35 w/w % of microcrystalline cellulose, for example, about 10 w/w %, about 11 w/w %, about 12 w/w %, about 13 w/w %, about 14 w/w %, about 15 w/w %, about 16 w/w %, about 17 w/w %, about 18 w/w %, about 19 w/w %, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, about 30 w/w %, about 31 w/w %, about 32 w/w %, about 33 w/w %, about 34 w/w %, or about 35 w/w % microcrystalline cellulose.

In some embodiments, the tablet comprises about 10 w/w % to about 20 w/w % of microcrystalline cellulose. In some embodiments, the tablet comprises about 10 w/w % to about 17 w/w % of microcrystalline cellulose. In some embodiments, the tablet comprises about 11 w/w % of microcrystalline cellulose. In some embodiments, the tablet comprises about 11.75 w/w % of microcrystalline cellulose. In some embodiments, the tablet comprises about 12 w/w % of microcrystalline cellulose. In some embodiments, the tablet comprises about 12.75 w/w % of microcrystalline cellulose. In some embodiments, the tablet comprises about 14 w/w % of microcrystalline cellulose. In some embodiments, the tablet comprises about 14.25 w/w % of microcrystalline cellulose. In some embodiments, the tablet comprises about 16 w/w % of microcrystalline cellulose. In some embodiments, the tablet comprises about 16.25 w/w % of microcrystalline cellulose.

In some embodiments, the tablet comprises about 20 w/w % to about 35 w/w % of microcrystalline cellulose. In some embodiments, the tablet comprises about 25 w/w % to about 35 w/w % of microcrystalline cellulose. In some embodiments, the tablet comprises about 25 w/w % to about 30 w/w % of microcrystalline cellulose. In some embodiments, the tablet comprises about 26 w/w % to about 29 w/w % of microcrystalline cellulose. In some embodiments, the tablet comprises about 27 w/w % to about 29 w/w % of microcrystalline cellulose. In some embodiments, the tablet comprises about 26.75 w/w % of microcrystalline cellulose. In some embodiments, the tablet comprises about 27 w/w % of microcrystalline cellulose. In some embodiments, the tablet comprises about 27.5 w/w % of microcrystalline cellulose. In some embodiments, the tablet comprises about 28 w/w % of microcrystalline cellulose. In some embodiments, the tablet comprises about 28.5 w/w % of microcrystalline cellulose.

In some embodiments, the tablet comprises about 1 w/w % to about 15 w/w % of crospovidone, for example, about 1 w/w %, about 2 w/w %, about 3 w/w %, about 4 w/w %, about 5 w/w %, about 6 w/w %, about 7 w/w %, about 8 w/w %, about 9 w/w %, about 10 w/w %, about 11 w/w %, about 12 w/w %, about 13 w/w %, about 14 w/w %, or about 15 w/w % crospovidone.

In some embodiments, the tablet comprises about 5 w/w % to about 15 w/w % of crospovidone. In some embodiments, the tablet comprises about 3 w/w % to about 11 w/w % of crospovidone. In some embodiments, the tablet comprises about 4 w/w % to about 10 w/w % of crospovidone. In some embodiments, the tablet comprises about 8 w/w % to about 12 w/w % of crospovidone. In some embodiments, the tablet comprises about 9 w/w % to about 11 w/w % of crospovidone. In some embodiments, the tablet comprises about 4 w/w % of crospovidone. In some embodiments, the tablet comprises about 5 w/w % of crospovidone. In some embodiments, the tablet comprises about 10 w/w % of crospovidone.

In some embodiments, the tablet comprises about 1 w/w % to about 5 w/w % of magnesium stearate, for example, about 1 w/w %, about 2 w/w %, about 3 w/w %, about 4 w/w %, or about 5 w/w % magnesium stearate. In some embodiments, the tablet comprises about 1 w/w % to about 3 w/w % of magnesium stearate. In some embodiments, the tablet comprises about 1 w/w % to about 2.5 w/w % of magnesium stearate. In some embodiments, the tablet comprises about 1.5 w/w % to about 2 w/w % of magnesium stearate. In some embodiments, the tablet comprises about 1.5 w/w % of magnesium stearate. In some embodiments, the tablet comprises about 2 w/w % of magnesium stearate.

In some embodiments, the tablet comprises or consists essentially of:
about 30 w/w % to about 80 w/w % of the compound of Formula Ia or Ib, or a pharmaceutically acceptable salt thereof;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

In some embodiments, the tablet comprises or consists essentially of:
about 30 w/w % to about 80 w/w % of the free acid form of the compound of Formula Ia or Ib;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

In some embodiments, the tablet comprises or consists essentially of:
about 35 w/w % to about 45 w/w % of the free acid form of the compound of Formula Ia or Ib;
about 25 w/w % to about 30 w/w % of mannitol;
about 25 w/w % to about 30 w/w % of microcrystalline cellulose;
about 3 w/w % to about 6 w/w % of crospovidone; and
about 1 w/w % to about 3 w/w % magnesium stearate.

In some embodiments, the tablet comprises or consists essentially of:
about 40 w/w % of the free acid form of the compound of Formula Ia or Ib;
about 27 w/w % of mannitol;
about 27.5 w/w % of microcrystalline cellulose;
about 4 w/w % of crospovidone; and about 1.5 w/w % of magnesium stearate.

In some embodiments, the tablet comprises or consists essentially of:
about 40 w/w % of the free acid form of the compound of Formula Ia or Ib;
about 26.75 w/w % of mannitol;
about 26.75 w/w % of microcrystalline cellulose;
about 5 w/w % of crospovidone; and about 1.5 w/w % of magnesium stearate.

In some embodiments, the tablet comprises or consists essentially of:
about 50 w/w % to about 80 w/w % of the compound of Formula Ia or Ib, or a pharmaceutically acceptable salt thereof;
about 10 w/w % to about 20 w/w % of mannitol;
about 10 w/w % to about 20 w/w % of microcrystalline cellulose;
about 5 w/w % to about 15 w/w % of crospovidone; and
about 1 w/w % to about 5 w/w % of magnesium stearate.

In some embodiments, the tablet comprises or consists essentially of:
about 55 w/w % to about 75 w/w % of the compound of Formula Ia or Ib, or a pharmaceutically acceptable salt thereof;
about 10 w/w % to about 20 w/w % of mannitol;
about 10 w/w % to about 20 w/w % of microcrystalline cellulose;
about 5 w/w % to about 15 w/w % of crospovidone; and
about 1 w/w % to about 3 w/w % of magnesium stearate.

In some embodiments, the tablet comprises or consists essentially of:
about 55 w/w % to about 75 w/w % of the compound of Formula Ia or Ib, or a pharmaceutically acceptable salt thereof;
about 10 w/w % to about 20 w/w % of mannitol;
about 10 w/w % to about 20 w/w % of microcrystalline cellulose;
about 5 w/w % to about 15 w/w % of crospovidone;
about 2 w/w % to about 4 w/w % poloxamer 407; and
about 1 w/w % to about 3 w/w % of magnesium stearate.

In some embodiments, the tablet comprises or consists essentially of:
about 50 w/w % to about 80 w/w % of the free acid form of the compound of Formula Ia or Ib;
about 10 w/w % to about 20 w/w % of mannitol;
about 10 w/w % to about 20 w/w % of microcrystalline cellulose;
about 5 w/w % to about 15 w/w % of crospovidone; and
about 1 w/w % to about 5 w/w % of magnesium stearate.

In some embodiments, the tablet comprises or consists essentially of:
about 55 w/w % to about 75 w/w % of the free acid form of the compound of Formula Ia or Ib;
about 10 w/w % to about 20 w/w % of mannitol;
about 10 w/w % to about 20 w/w % of microcrystalline cellulose;
about 5 w/w % to about 15 w/w % of crospovidone; and
about 1 w/w % to about 3 w/w % of magnesium stearate.

In some embodiments, the tablet comprises or consists essentially of:
about 55 w/w % to about 75 w/w % of the free acid form of the compound of Formula Ia or Ib;
about 10 w/w % to about 20 w/w % of mannitol;
about 10 w/w % to about 20 w/w % of microcrystalline cellulose;
about 5 w/w % to about 15 w/w % of crospovidone;
about 2 w/w % to about 4 w/w % poloxamer 407; and
about 1 w/w % to about 3 w/w % of magnesium stearate.

In some embodiments, the tablet comprises or consists essentially of:
about 60 w/w % of the free acid form of the compound of Formula Ia or Ib;
about 16.75 w/w % of mannitol;
about 16.75 w/w % of microcrystalline cellulose;
about 5 w/w % of crospovidone; and
about 1.5 w/w % of magnesium stearate.

In some embodiments, the tablet comprises or consists essentially of:
about 70 w/w % of the free acid form of the compound of Formula Ia or Ib;
about 11.75 w/w % of mannitol;
about 11.75 w/w % of microcrystalline cellulose;
about 5 w/w % of crospovidone; and about 1.5 w/w % of magnesium stearate.

In some embodiments, the tablet comprises or consists essentially of:
about 60 w/w % of the free acid form of the compound of Formula Ia or Ib;
about 14.25 w/w % of mannitol;
about 14.25 w/w % of microcrystalline cellulose;
about 10 w/w % of crospovidone; and about 1.5 w/w % of magnesium stearate.

In some embodiments, the tablet comprises or consists essentially of:
about 60 w/w % of the free acid form of the compound of Formula Ia or Ib;
about 12.75 w/w % of mannitol;
about 12.75 w/w % of microcrystalline cellulose;
about 10 w/w % of crospovidone;
about 3 w/w % poloxamer 407; and about 1.5 w/w % of magnesium stearate.

In some embodiments, the tablet comprises or consists essentially of:
about 50 w/w % to about 70 w/w % of the compound of Formula Ia or Ib, or a pharmaceutically acceptable salt thereof;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the tablet may comprise or consist essentially of about 300 mg or about 700 mg of the compound of Formula Ia or Ib, or a pharmaceutically acceptable salt thereof.

In some embodiments, the tablet comprises or consists essentially of:
about 55 w/w % to about 65 w/w % of the compound of Formula Ia or Ib, or a pharmaceutically acceptable salt thereof;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the tablet may comprise or consist essentially of about 300 mg or about 700 mg of the compound of Formula Ia or Ib, or a pharmaceutically acceptable salt thereof.

In some embodiments, the tablet comprises or consists essentially of:
about 60 w/w % of the compound of Formula Ia or Ib, or a pharmaceutically acceptable salt thereof;
about 28.5 w/w % of microcrystalline cellulose;
about 10 w/w % of crospovidone; and
about 1.5 w/w % of magnesium stearate.

In some embodiments, the tablet comprises or consists essentially of:
about 60 w/w % of the compound of Formula Ia or Ib, or a pharmaceutically acceptable salt thereof;
about 28 w/w % of microcrystalline cellulose;
about 10 w/w % of crospovidone; and
about 2 w/w % of magnesium stearate.

In some embodiments, the tablet may comprise or consist essentially of about 300 mg or about 700 mg of the compound of Formula Ia or Ib, or a pharmaceutically acceptable salt thereof. In some embodiments, the tablet comprises or consists essentially of:
about 50 w/w % to about 70 w/w % of the free acid form of the compound of Formula Ia or Ib;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the tablet comprises or consists essentially of:
about 55 w/w % to about 65 w/w % of the free acid form of the compound of Formula Ia or Ib;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the tablet comprises or consists essentially of:
about 60 w/w % of the free acid form of the compound of Formula Ia or Ib;
about 28.5 w/w % of microcrystalline cellulose;
about 10 w/w % of crospovidone; and
about 1.5 w/w % of magnesium stearate.

In some embodiments, the tablet comprises or consists essentially of:
about 60 w/w % of the free acid form of the compound of Formula Ia or Ib;
about 28 w/w % of microcrystalline cellulose;
about 10 w/w % of crospovidone; and about 2 w/w % of magnesium stearate.

In some embodiments, the tablet comprises or consists essentially of:
about 50 w/w % to about 70 w/w % of an amorphous form of the free acid form of the compound of Formula Ia or Ib;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the tablet comprises or consists essentially of:
about 55 w/w % to about 65 w/w % of an amorphous form of the free acid form of the compound of Formula Ia or Ib;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the tablet comprises or consists essentially of:
about 60 w/w % of an amorphous form of the free acid form of the compound of Formula Ia or Ib;
about 28.5 w/w % of microcrystalline cellulose;
about 10 w/w % of crospovidone; and
about 1.5 w/w % of magnesium stearate.

In some embodiments, the tablet comprises or consists essentially of:
about 60 w/w % of an amorphous form of the free acid form of the compound of Formula Ia or Ib;
about 28 w/w % of microcrystalline cellulose;
about 10 w/w % of crospovidone; and
about 2 w/w % of magnesium stearate.

In some embodiments, the tablet comprises or consists essentially of:
about 50 w/w % to about 70 w/w % of the compound of Formula Ia, or a pharmaceutically acceptable salt thereof;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the tablet comprises or consists essentially of:
about 55 w/w % to about 65 w/w % of the compound of Formula Ia, or a pharmaceutically acceptable salt thereof;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the tablet comprises or consists essentially of:
about 60 w/w % of the compound of Formula Ia, or a pharmaceutically acceptable salt thereof;
about 28.5 w/w % of microcrystalline cellulose;
about 10 w/w % of crospovidone; and
about 1.5 w/w % of magnesium stearate.

In some embodiments, the tablet comprises or consists essentially of:
about 60 w/w % of the compound of Formula Ia, or a pharmaceutically acceptable salt thereof;
about 28 w/w % of microcrystalline cellulose;
about 10 w/w % of crospovidone; and
about 2 w/w % of magnesium stearate.

In some embodiments, the tablet comprises or consists essentially of:
about 50 w/w % to about 70 w/w % of the free acid form of the compound of Formula Ia;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the tablet comprises or consists essentially of:
about 55 w/w % to about 65 w/w % of the free acid form of the compound of Formula Ia;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the tablet comprises or consists essentially of:
about 60 w/w % of the free acid form of the compound of Formula Ia;
about 28.5 w/w % of microcrystalline cellulose;
about 10 w/w % of crospovidone; and
about 1.5 w/w % of magnesium stearate.

In some embodiments, the tablet comprises or consists essentially of:
about 60 w/w % of the free acid form of the compound of Formula Ia;
about 28 w/w % of microcrystalline cellulose;
about 10 w/w % of crospovidone; and about 2 w/w % of magnesium stearate.

In some embodiments, the tablet comprises or consists essentially of:
about 50 w/w % to about 70 w/w % of an amorphous form of the free acid form of the compound of Formula Ia;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the tablet comprises or consists essentially of:
about 55 w/w % to about 65 w/w % of an amorphous form of the free acid form of the compound of Formula Ia;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the tablet comprises or consists essentially of:
about 60 w/w % of an amorphous form of the free acid form of the compound of Formula Ia;
about 28.5 w/w % of microcrystalline cellulose;
about 10 w/w % of crospovidone; and
about 1.5 w/w % of magnesium stearate.

In some embodiments, the tablet comprises or consists essentially of:
about 60 w/w % of an amorphous form of the free acid form of the compound of Formula Ia;
about 28 w/w % of microcrystalline cellulose;
about 10 w/w % of crospovidone; and
about 2 w/w % of magnesium stearate.

In some embodiments, the tablet comprises or consists essentially of:
about 50 w/w % to about 70 w/w % of the compound of Formula Ib, or a pharmaceutically acceptable salt thereof;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the tablet may comprise or consist essentially of about 300 mg or about 700 mg of the compound of Formula Ib, or a pharmaceutically acceptable salt thereof.

In some embodiments, the tablet comprises or consists essentially of:
about 55 w/w % to about 65 w/w % of the compound of Formula Ib, or a pharmaceutically acceptable salt thereof;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the tablet may comprise or consist essentially of about 300 mg or about 700 mg of the compound of Formula Ib, or a pharmaceutically acceptable salt thereof.

In some embodiments, the tablet comprises or consists essentially of:
about 60 w/w % of the compound of Formula Ib, or a pharmaceutically acceptable salt thereof;
about 28.5 w/w % of microcrystalline cellulose;
about 10 w/w % of crospovidone; and
about 1.5 w/w % of magnesium stearate.

In some embodiments, the tablet comprises or consists essentially of:
about 60 w/w % of the compound of Formula Ib, or a pharmaceutically acceptable salt thereof;
about 28 w/w % of microcrystalline cellulose;
about 10 w/w % of crospovidone; and
about 2 w/w % of magnesium stearate.

In some embodiments, the tablet may comprise or consist essentially of about 300 mg or about 700 mg of the compound of Formula Ib, or a pharmaceutically acceptable salt thereof.

In some embodiments, the tablet comprises or consists essentially of:
about 50 w/w % to about 70 w/w % of the free acid form of the compound of Formula Ib;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the tablet may comprise or consist essentially of about 300 mg or about 700 mg of the free acid form of the compound of Formula Ib.

In some embodiments, the tablet comprises or consists essentially of:
about 55 w/w % to about 65 w/w % of the free acid form of the compound of Formula Ib; about 25 w/w % to about 30 w/w % of microcrystalline cellulose;
about 8 w/w % to about 12 w/w % of crospovidone; and about 1 w/w % to about 3 w/w % of magnesium stearate.

In some embodiments, the tablet may comprise or consist essentially of about 300 mg or about 700 mg of the free acid form of the compound of Formula Ib.

In some embodiments, the tablet comprises or consists essentially of:
about 60 w/w % of the free acid form of the compound of Formula Ib;
about 28.5 w/w % of microcrystalline cellulose;
about 10 w/w % of crospovidone; and about 1.5 w/w % of magnesium stearate.

In some embodiments, the tablet comprises or consists essentially of:
about 60 w/w % of the free acid form of the compound of Formula Ib;
about 28 w/w % of microcrystalline cellulose;
about 10 w/w % of crospovidone; and about 2 w/w % of magnesium stearate.

In some embodiments, the tablet may comprise or consist essentially of about 300 mg or about 700 mg of the free acid form of the compound of Formula Ib.

In some embodiments, the tablet comprises or consists essentially of:
about 50 w/w % to about 70 w/w % of an amorphous form of the free acid form of the compound of Formula Ib;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the tablet may comprise or consist essentially of about 300 mg or about 700 mg of the amorphous form of the free acid form of the compound of Formula Ib.

In some embodiments, the tablet comprises or consists essentially of:
about 55 w/w % to about 65 w/w % of an amorphous form of the free acid form of the compound of Formula Ib;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the tablet may comprise or consist essentially of about 300 mg or about 700 mg of the amorphous form of the free acid form of the compound of Formula Ib.

In some embodiments, the tablet comprises or consists essentially of:
about 60 w/w % of an amorphous form of the free acid form of the compound of Formula Ib;
about 28.5 w/w % of microcrystalline cellulose;
about 10 w/w % of crospovidone; and
about 1.5 w/w % of magnesium stearate.

In some embodiments, the tablet comprises or consists essentially of:
about 60 w/w % of an amorphous form of the free acid form of the compound of Formula Ib;
about 28 w/w % of microcrystalline cellulose;
about 10 w/w % of crospovidone; and
about 2 w/w % of magnesium stearate.

In some embodiments, the tablet may comprise or consist essentially of about 300 mg or about 700 mg of the amorphous form of the free acid form of the compound of Formula Ib.

In some embodiments, the tablet comprises about 1 mg to about 2000 mg of the compound of Formula Ia or Ib, or a pharmaceutically acceptable salt thereof, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, or about 2000 mg of the compound of Formula Ia or Ib, or a pharmaceutically acceptable salt thereof.

In some embodiments, the tablet comprises about 225 mg to about 375 mg of the compound of Formula Ia or Ib, or a pharmaceutically acceptable salt thereof. In some embodiments, the tablet comprises about 250 mg to about 375 mg of the compound of Formula Ia or Ib, or a pharmaceutically acceptable salt thereof. In some embodiments, the tablet comprises about 275 mg to about 375 mg of the compound of Formula Ia or Ib, or a pharmaceutically acceptable salt thereof. In some embodiments, the tablet comprises about 300 mg to about 350 mg of the compound of Formula Ia or Ib, or a pharmaceutically acceptable salt thereof.

In some embodiments, the tablet comprises about 300 mg to about 310 mg of the compound of Formula Ia or Ib, or a pharmaceutically acceptable salt thereof. In some embodiments, the tablet comprises about 305 mg to about 307 mg of the compound of Formula Ia or Ib, or a pharmaceutically acceptable salt thereof. In some embodiments, the tablet comprises about 306 mg of the compound of Formula Ia or Ib, or a pharmaceutically acceptable salt thereof. In some embodiments, the tablet comprises about 306 mg of the free acid form of the compound of Formula Ia or Ib. In some embodiments, the tablet comprises about 306 mg of the free acid form of the compound of Formula Ia. In some embodiments, the tablet comprises about 306 mg of the free acid form of the compound of Formula Ib. In some embodiments, the tablet comprises about 306 mg of an amorphous form of the free acid form of the compound of Formula Ia. In some embodiments, the tablet comprises about 306 mg of an amorphous form of the free acid form of the compound of Formula Ib. In some embodiments, the tablet comprises about 300 mg of the compound of Formula Ia or Ib, or a pharmaceutically acceptable salt thereof. In some embodiments, the tablet comprises about 300 mg of the free acid form of the compound of Formula Ia or Ib. In some embodiments, the tablet comprises about 300 mg of the free acid form of the compound of Formula Ia. In some embodiments, the tablet comprises about 300 mg of the free acid form of the compound of Formula Ib. In some embodiments, the tablet comprises about 300 mg of an amorphous form of the free acid form of the compound of Formula Ia. In some embodiments, the tablet comprises about 300 mg of an amorphous form of the free acid form of the compound of Formula Ib.

In some embodiments, the tablet comprises about 100 mg to about 500 mg of the compound of Formula Ia or Ib, or a pharmaceutically acceptable salt thereof. In some embodiments, the tablet comprises about 200 mg to about 475 mg of the compound of Formula Ia or Ib, or a pharmaceutically acceptable salt thereof. In some embodiments, the tablet comprises about 200 mg to about 450 mg of the compound of Formula Ia or Ib, or a pharmaceutically acceptable salt thereof. In some embodiments, the tablet comprises about 250 mg to about 450 mg of the compound of Formula Ia or Ib, or a pharmaceutically acceptable salt thereof. In some embodiments, the tablet comprises about 300 mg to about 400 mg of the compound of Formula Ia or Ib, or a pharmaceutically acceptable salt thereof. In some embodiments, the tablet comprises about 325 mg to about 375 mg of the compound of Formula Ia or Ib, or a pharmaceutically acceptable salt thereof. In some embodiments, the tablet comprises about 340 mg to about 360 mg of the compound of Formula Ia or Ib, or a pharmaceutically acceptable salt thereof. In some embodiments, the tablet comprises about 345 mg to about 355 mg of the compound of Formula Ia or Ib, or a pharmaceutically acceptable salt thereof. In some embodiments, the tablet comprises about 349 mg to about 351 mg of the compound of Formula Ia or Ib, or a pharmaceutically acceptable salt thereof. In some embodiments, the tablet comprises about 350 mg of the compound of Formula Ia or Ib, or a pharmaceutically acceptable salt thereof.

In some embodiments, the tablet comprises about 100 mg to about 500 mg of the free acid form of the compound of Formula Ia or Ib. In some embodiments, the tablet comprises about 200 mg to about 475 mg of the free acid form of the compound of Formula Ia or Ib. In some embodiments, the tablet comprises about 200 mg to about 450 mg of the free acid form of the compound of Formula Ia or Ib. In some embodiments, the tablet comprises about 250 mg to about 450 mg of the free acid form of the compound of Formula Ia or Ib. In some embodiments, the tablet comprises about 300 mg to about 400 mg of the free acid form of the compound of Formula Ia or Ib. In some embodiments, the tablet comprises about 325 mg to about 375 mg of the free acid form of the compound of Formula Ia or Ib. In some embodiments, the tablet comprises about 340 mg to about 360 mg of the free acid form of the compound of Formula Ia or Ib. In some embodiments, the tablet comprises about 345 mg to about 355 mg of the free acid form of the compound of Formula Ia or Ib. In some embodiments, the tablet comprises about 349 mg to about 351 mg of the free acid form of the compound of Formula Ia or Ib. In some embodiments, the tablet comprises about 350 mg of the compound of the free acid form of the compound of Formula Ia or Ib.

In some embodiments, the tablet comprises about 100 mg to about 500 mg of the free acid form of the compound of Formula Ia. In some embodiments, the tablet comprises about 200 mg to about 475 mg of the free acid form of the compound of Formula Ia. In some embodiments, the tablet comprises about 200 mg to about 450 mg of the free acid form of the compound of Formula Ia. In some embodiments, the tablet comprises about 250 mg to about 450 mg of the free acid form of the compound of Formula Ia. In some embodiments, the tablet comprises about 300 mg to about 400 mg of the free acid form of the compound of Formula Ia. In some embodiments, the tablet comprises about 325 mg to about 375 mg of the free acid form of the compound of Formula Ia. In some embodiments, the tablet comprises about 340 mg to about 360 mg of the free acid form of the compound of Formula Ia. In some embodiments, the tablet comprises about 345 mg to about 355 mg of the free acid form of the compound of Formula Ia. In some embodiments, the tablet comprises about 349 mg to about 351 mg of the free acid form of the compound of Formula Ia. In some embodiments, the tablet comprises about 350 mg of the compound of the free acid form of the compound of Formula Ia.

In some embodiments, the tablet comprises about 100 mg to about 500 mg of the free acid form of the compound of Formula Ib. In some embodiments, the tablet comprises about 200 mg to about 475 mg of the free acid form of the compound of Formula Ib. In some embodiments, the tablet comprises about 200 mg to about 450 mg of the free acid form of the compound of Formula Ib. In some embodiments, the tablet comprises about 250 mg to about 450 mg of the free acid form of the compound of Formula Ib. In some embodiments, the tablet comprises about 300 mg to about 400 mg of the free acid form of the compound of Formula Ib. In some embodiments, the tablet comprises about 325 mg to about 375 mg of the free acid form of the compound of Formula Ib. In some embodiments, the tablet comprises about 340 mg to about 360 mg of the free acid form of the compound of Formula Ib. In some embodiments, the tablet comprises about 345 mg to about 355 mg of the free acid form of the compound of Formula Ib. In some embodiments, the tablet comprises about 349 mg to about 351 mg of the free acid form of the compound of Formula Ib. In some embodiments, the tablet comprises about 350 mg of the compound of the free acid form of the compound of Formula Ib.

In some embodiments, the tablet comprises about 100 mg to about 500 mg of an amorphous form of the free acid form of the compound of Formula Ia or Ib. In some embodiments, the tablet comprises about 200 mg to about 475 mg of the free acid form of an amorphous form of the free acid form of the compound of Formula Ia or Ib. In some embodiments, the tablet comprises about 200 mg to about 450 mg of an amorphous form of the free acid form of the compound of Formula Ia or Ib. In some embodiments, the tablet comprises about 250 mg to about 450 mg of an amorphous form of the free acid form of the compound of Formula Ia or Ib. In some embodiments, the tablet comprises about 300 mg to about 400 mg of an amorphous form of the free acid form of the compound of Formula Ia or Ib. In some embodiments, the tablet comprises about 325 mg to about 375 mg of an amorphous form of the free acid form of the compound of Formula Ia or Ib. In some embodiments, the tablet comprises about 340 mg to about 360 mg of an amorphous form of the free acid form of the compound of Formula Ia or Ib. In some embodiments, the tablet comprises about 345 mg to about 355 mg of an amorphous form of the free acid form of the compound of Formula Ia or Ib. In some embodiments, the tablet comprises about 349 mg to about 351 mg of an amorphous form of the free acid form of the compound of Formula Ia or Ib. In some embodiments, the tablet comprises about 350 mg of the compound of an amorphous form of the free acid form of the compound of Formula Ia or Ib.

In some embodiments, the tablet comprises about 100 mg to about 500 mg of an amorphous form of the free acid form of the compound of Formula Ia. In some embodiments, the tablet comprises about 200 mg to about 475 mg of the free acid form of an amorphous form of the free acid form of the compound of Formula Ia. In some embodiments, the tablet comprises about 200 mg to about 450 mg of an amorphous form of the free acid form of the compound of Formula Ia. In some embodiments, the tablet comprises about 250 mg to about 450 mg of an amorphous form of the free acid form of the compound of Formula Ia. In some embodiments, the tablet comprises about 300 mg to about 400 mg of an amorphous form of the free acid form of the compound of Formula Ia. In some embodiments, the tablet comprises about 325 mg to about 375 mg of an amorphous form of the free acid form of the compound of Formula Ia. In some embodiments, the tablet comprises about 340 mg to about 360 mg of an amorphous form of the free acid form of the compound of Formula Ia. In some embodiments, the tablet comprises about 345 mg to about 355 mg of an amorphous form of the free acid form of the compound of Formula Ia. In some embodiments, the tablet comprises about 349 mg to about 351 mg of an amorphous form of the free acid form of the compound of Formula Ia. In some embodiments, the tablet comprises about 350 mg of the compound of an amorphous form of the free acid form of the compound of Formula Ia.

In some embodiments, the tablet comprises about 100 mg to about 500 mg of an amorphous form of the free acid form of the compound of Formula Ib. In some embodiments, the tablet comprises about 200 mg to about 475 mg of the free acid form of an amorphous form of the free acid form of the compound of Formula Ib. In some embodiments, the tablet comprises about 200 mg to about 450 mg of an amorphous form of the free acid form of the compound of Formula Ib. In some embodiments, the tablet comprises about 250 mg to about 450 mg of an amorphous form of the free acid form of the compound of Formula Ib. In some embodiments, the tablet comprises about 300 mg to about 400 mg of an amorphous form of the free acid form of the compound of Formula Ib. In some embodiments, the tablet comprises about 325 mg to about 375 mg of an amorphous form of the free acid form of the compound of Formula Ib. In some embodiments, the tablet comprises about 340 mg to about 360 mg of an amorphous form of the free acid form of the compound of Formula Ib. In some embodiments, the tablet comprises about 345 mg to about 355 mg of an amorphous form of the free acid form of the compound of Formula Ib. In some embodiments, the tablet comprises about 349 mg to about 351 mg of an amorphous form of the free acid form of the compound of Formula Ib. In some embodiments, the tablet comprises about 350 mg of the compound of an amorphous form of the free acid form of the compound of Formula Ib.

In some embodiments, the tablet comprises about 500 mg to about 1000 mg of the compound of Formula Ia or Ib, or a pharmaceutically acceptable salt thereof. In some embodiments, the tablet comprises about 600 mg to about 800 mg of the compound of Formula Ia or Ib, or a pharmaceutically acceptable salt thereof. In some embodiments, the tablet comprises about 600 mg to about 750 mg of the compound of Formula Ia or Ib, or a pharmaceutically acceptable salt thereof. In some embodiments, the tablet comprises about 650 mg to about 750 mg of the compound of Formula Ia or Ib, or a pharmaceutically acceptable salt thereof. In some embodiments, the tablet comprises about 675 mg to about 725 mg of the compound of Formula Ia or Ib, or a pharmaceutically acceptable salt thereof. In some embodiments, the tablet comprises about 690 mg to about 710 mg of the compound of Formula Ia or Ib, or a pharmaceutically acceptable salt thereof. In some embodiments, the tablet comprises about 695 mg to about 705 mg of the compound of Formula Ia or Ib, or a pharmaceutically acceptable salt thereof. In some embodiments, the tablet comprises about 699 mg to about 701 mg of the compound of Formula Ia or Ib, or a pharmaceutically acceptable salt thereof. In some embodiments, the tablet comprises about 700 mg of the compound of Formula Ia or Ib, or a pharmaceutically acceptable salt thereof.

In some embodiments, the tablet comprises about 500 mg to about 1000 mg of the free acid form of the compound of Formula Ia or Ib. In some embodiments, the tablet comprises about 600 mg to about 800 mg of the free acid form of the compound of Formula Ia or Ib. In some embodiments, the tablet comprises about 600 mg to about 750 mg of the free acid form of the compound of Formula Ia or Ib. In some embodiments, the tablet comprises about 650 mg to about 750 mg of the free acid form of the compound of Formula Ia or Ib. In some embodiments, the tablet comprises about 675 mg to about 725 mg of the free acid form of the compound of Formula Ia or Ib. In some embodiments, the tablet comprises about 690 mg to about 710 mg of the free acid form of the compound of Formula Ia or Ib. In some embodiments, the tablet comprises about 695 mg to about 705 mg of the free acid form of the compound of Formula Ia or Ib. In some embodiments, the tablet comprises about 699 mg to about 701 mg of the free acid form of the compound of Formula Ia or Ib. In some embodiments, the tablet comprises about 700 mg of the free acid form of the compound of Formula Ia or Ib.

In some embodiments, the tablet comprises about 500 mg to about 1000 mg of the free acid form of the compound of Formula Ia. In some embodiments, the tablet comprises about 600 mg to about 800 mg of the free acid form of the compound of Formula Ia. In some embodiments, the tablet comprises about 600 mg to about 750 mg of the free acid form of the compound of Formula Ia. In some embodiments, the tablet comprises about 650 mg to about 750 mg of the free acid form of the compound of Formula Ia. In some embodiments, the tablet comprises about 675 mg to about 725 mg of the free acid form of the compound of Formula Ia. In some embodiments, the tablet comprises about 690 mg to about 710 mg of the free acid form of the compound of Formula Ia. In some embodiments, the tablet comprises about 695 mg to about 705 mg of the free acid form of the compound of Formula Ia. In some embodiments, the tablet comprises about 699 mg to about 701 mg of the free acid form of the compound of Formula Ia. In some embodiments, the tablet comprises about 700 mg of the free acid form of the compound of Formula Ia.

In some embodiments, the tablet comprises about 500 mg to about 1000 mg of the free acid form of the compound of Formula Ib. In some embodiments, the tablet comprises about 600 mg to about 800 mg of the free acid form of the compound of Formula Ib. In some embodiments, the tablet comprises about 600 mg to about 750 mg of the free acid form of the compound of Formula Ib. In some embodiments, the tablet comprises about 650 mg to about 750 mg of the free acid form of the compound of Formula Ib. In some embodiments, the tablet comprises about 675 mg to about 725 mg of the free acid form of the compound of Formula Ib. In some embodiments, the tablet comprises about 690 mg to about 710 mg of the free acid form of the compound of Formula Ib. In some embodiments, the tablet comprises about 695 mg to about 705 mg of the free acid form of the compound of Formula Ib. In some embodiments, the tablet comprises about 699 mg to about 701 mg of the free acid form of the compound of Formula Ib. In some embodiments, the tablet comprises about 700 mg of the free acid form of the compound of Formula Ib.

In some embodiments, the tablet comprises about 500 mg to about 1000 mg of an amorphous form of the free acid form of the compound of Formula Ia or Ib. In some embodiments, the tablet comprises about 600 mg to about 800 mg of an amorphous form of the free acid form of the compound of Formula Ia or Ib. In some embodiments, the tablet comprises about 600 mg to about 750 mg of an amorphous form of the free acid form of the compound of Formula Ia or Ib. In some embodiments, the tablet comprises about 650 mg to about 750 mg of an amorphous form of the free acid form of the compound of Formula Ia or Ib. In some embodiments, the tablet comprises about 675 mg to about 725 mg of an amorphous form of the free acid form of the compound of Formula Ia or Ib. In some embodiments, the tablet comprises about 690 mg to about 710 mg of an amorphous form of the free acid form of the compound of Formula Ia or Ib. In some embodiments, the tablet comprises about 695 mg to about 705 mg of an amorphous form of the free acid form of the compound of Formula Ia or Ib. In some embodiments, the tablet comprises about 699 mg to about 701 mg of an amorphous form of the free acid form of the compound of Formula Ia or Ib. In some embodiments, the tablet comprises about 700 mg of an amorphous form of the free acid form of the compound of Formula Ia or Ib.

In some embodiments, the tablet comprises about 500 mg to about 1000 mg of an amorphous form of the free acid form of the compound of Formula Ia. In some embodiments, the tablet comprises about 600 mg to about 800 mg of an amorphous form of the free acid form of the compound of Formula Ia. In some embodiments, the tablet comprises about 600 mg to about 750 mg of an amorphous form of the free acid form of the compound of Formula Ia. In some embodiments, the tablet comprises about 650 mg to about 750 mg of an amorphous form of the free acid form of the compound of Formula Ia. In some embodiments, the tablet comprises about 675 mg to about 725 mg of an amorphous form of the free acid form of the compound of Formula Ia. In some embodiments, the tablet comprises about 690 mg to about 710 mg of an amorphous form of the free acid form of the compound of Formula Ia. In some embodiments, the tablet comprises about 695 mg to about 705 mg of an amorphous form of the free acid form of the compound of Formula Ia. In some embodiments, the tablet comprises about 699 mg to about 701 mg of an amorphous form of the free acid form of the compound of Formula Ia. In some embodiments, the tablet comprises about 700 mg of an amorphous form of the free acid form of the compound of Formula Ia.

In some embodiments, the tablet comprises about 500 mg to about 1000 mg of an amorphous form of the free acid form of the compound of Formula Ib. In some embodiments, the tablet comprises about 600 mg to about 800 mg of an amorphous form of the free acid form of the compound of Formula Ib. In some embodiments, the tablet comprises about 600 mg to about 750 mg of an amorphous form of the free acid form of the compound of Formula Ib. In some embodiments, the tablet comprises about 650 mg to about 750 mg of an amorphous form of the free acid form of the compound of Formula Ib. In some embodiments, the tablet comprises about 675 mg to about 725 mg of an amorphous form of the free acid form of the compound of Formula Ib. In some embodiments, the tablet comprises about 690 mg to about 710 mg of an amorphous form of the free acid form of the compound of Formula Ib. In some embodiments, the tablet comprises about 695 mg to about 705 mg of an amorphous form of the free acid form of the compound of Formula Ib. In some embodiments, the tablet comprises about 699 mg to about 701 mg of an amorphous form of the free acid form of the compound of Formula Ib. In some embodiments, the tablet comprises about 700 mg of an amorphous form of the free acid form of the compound of Formula Ib.

In some embodiments, the tablet comprises about 800 mg to about 1000 mg of the compound of Formula Ia or Ib, or a pharmaceutically acceptable salt thereof. In some embodiments, the tablet comprises about 650 mg to about 1000 mg of the compound of Formula Ia or Ib, or a pharmaceutically acceptable salt thereof. In some embodiments, the tablet comprises about 800 mg to about 1000 mg of the compound of Formula Ia or Ib, or a pharmaceutically acceptable salt thereof. In some embodiments, the tablet comprises about 875 mg to about 925 mg of the compound of Formula Ia or Ib, or a pharmaceutically acceptable salt thereof. In some embodiments, the tablet comprises about 890 mg to about 910 mg of the compound of Formula Ia or Ib, or a pharmaceutically acceptable salt thereof. In some embodiments, the tablet comprises about 895 mg to about 905 mg of the compound of Formula Ia or Ib, or a pharmaceutically acceptable salt thereof. In some embodiments, the tablet comprises about 899 mg to about 901 mg of the compound of Formula Ia or Ib, or a pharmaceutically acceptable salt thereof. In some embodiments, the tablet comprises about 900 mg of the compound of Formula Ia or Ib, or a pharmaceutically acceptable salt thereof.

In some embodiments, the tablet comprises about 800 mg to about 1000 mg of the compound of Formula Ia or Ib, or a pharmaceutically acceptable salt thereof. In some embodiments, the tablet comprises about 650 mg to about 1000 mg of the compound of Formula Ia or Ib, or a pharmaceutically acceptable salt thereof. In some embodiments, the tablet comprises about 800 mg to about 1000 mg of the compound of Formula Ia or Ib, or a pharmaceutically acceptable salt thereof. In some embodiments, the tablet comprises about 875 mg to about 925 mg of the compound of Formula Ia or Ib, or a pharmaceutically acceptable salt thereof. In some embodiments, the tablet comprises about 890 mg to about 910 mg of the compound of Formula Ia or Ib, or a pharmaceutically acceptable salt thereof. In some embodiments, the tablet comprises about 895 mg to about 905 mg of the compound of Formula Ia or Ib, or a pharmaceutically acceptable salt thereof. In some embodiments, the tablet comprises about 899 mg to about 901 mg of the compound of Formula Ia or Ib, or a pharmaceutically acceptable salt thereof. In some embodiments, the tablet comprises about 900 mg of the compound of Formula Ia or Ib, or a pharmaceutically acceptable salt thereof.

In some embodiments, the tablet comprises about 800 mg to about 1000 mg of the free acid form of the compound of Formula Ia or Ib. In some embodiments, the tablet comprises about 650 mg to about 1000 mg of the free acid form of the compound of Formula Ia or Ib. In some embodiments, the tablet comprises about 800 mg to about 1000 mg of the free acid form of the compound of Formula Ia or Ib. In some embodiments, the tablet comprises about 875 mg to about 925 mg of the free acid form of the compound of Formula Ia or Ib. In some embodiments, the tablet comprises about 890 mg to about 910 mg of the free acid form of the compound of Formula Ia or Ib. In some embodiments, the tablet comprises about 895 mg to about 905 mg of the free acid form of the compound of Formula Ia or Ib. In some embodiments, the tablet comprises about 899 mg to about 901 mg of the free acid form of the compound of Formula Ia or Ib. In some embodiments, the tablet comprises about 900 mg of the free acid form of the compound of Formula Ia or Ib.

In some embodiments, the tablet comprises about 800 mg to about 1000 mg of the free acid form of the compound of Formula Ia. In some embodiments, the tablet comprises about 650 mg to about 1000 mg of the free acid form of the compound of Formula Ia. In some embodiments, the tablet comprises about 800 mg to about 1000 mg of the free acid form of the compound of Formula Ia. In some embodiments, the tablet comprises about 875 mg to about 925 mg of the free acid form of the compound of Formula Ia. In some embodiments, the tablet comprises about 890 mg to about 910 mg of the free acid form of the compound of Formula Ia. In some embodiments, the tablet comprises about 895 mg to about 905 mg of the free acid form of the compound of Formula Ia. In some embodiments, the tablet comprises about 899 mg to about 901 mg of the free acid form of the compound of Formula Ia. In some embodiments, the tablet comprises about 900 mg of the free acid form of the compound of Formula Ia.

In some embodiments, the tablet comprises about 800 mg to about 1000 mg of the free acid form of the compound of Formula Ib. In some embodiments, the tablet comprises about 650 mg to about 1000 mg of the free acid form of the compound of Formula Ib. In some embodiments, the tablet comprises about 800 mg to about 1000 mg of the free acid form of the compound of Formula Ib. In some embodiments, the tablet comprises about 875 mg to about 925 mg of the free acid form of the compound of Formula Ib. In some embodiments, the tablet comprises about 890 mg to about 910 mg of the free acid form of the compound of Formula Ib. In some embodiments, the tablet comprises about 895 mg to about 905 mg of the free acid form of the compound of Formula Ib. In some embodiments, the tablet comprises about 899 mg to about 901 mg of the free acid form of the compound of Formula Ib. In some embodiments, the tablet comprises about 900 mg of the free acid form of the compound of Formula Ib.

In some embodiments, the tablet comprises about 800 mg to about 1000 mg of an amorphous form of the free acid form of the compound of Formula Ia or Ib. In some embodiments, the tablet comprises about 650 mg to about 1000 mg of an amorphous form of the free acid form of the compound of Formula Ia or Ib. In some embodiments, the tablet comprises about 800 mg to about 1000 mg of an amorphous form of the free acid form of the compound of Formula Ia or Ib. In some embodiments, the tablet comprises about 875 mg to about 925 mg of an amorphous form of the free acid form of the compound of Formula Ia or Ib. In some embodiments, the tablet comprises about 890 mg to about 910 mg of an amorphous form of the free acid form of the compound of Formula Ia or Ib. In some embodiments, the tablet comprises about 895 mg to about 905 mg of an amorphous form of the free acid form of the compound of Formula Ia or Ib. In some embodiments, the tablet comprises about 899 mg to about 901 mg of an amorphous form of the free acid form of the compound of Formula Ia or Ib. In some embodiments, the tablet comprises about 900 mg of an amorphous form of the free acid form of the compound of Formula Ia or Ib.

In some embodiments, the tablet comprises about 800 mg to about 1000 mg of an amorphous form of the free acid form of the compound of Formula Ia. In some embodiments, the tablet comprises about 650 mg to about 1000 mg of an amorphous form of the free acid form of the compound of Formula Ia. In some embodiments, the tablet comprises about 800 mg to about 1000 mg of an amorphous form of the free acid form of the compound of Formula Ia. In some embodiments, the tablet comprises about 875 mg to about 925 mg of an amorphous form of the free acid form of the compound of Formula Ia. In some embodiments, the tablet comprises about 890 mg to about 910 mg of an amorphous form of the free acid form of the compound of Formula Ia. In some embodiments, the tablet comprises about 895 mg to about 905 mg of an amorphous form of the free acid form of the compound of Formula Ia. In some embodiments, the tablet comprises about 899 mg to about 901 mg of an amorphous form of the free acid form of the compound of Formula Ia. In some embodiments, the tablet comprises about 900 mg of an amorphous form of the free acid form of the compound of Formula Ia.

In some embodiments, the tablet comprises about 800 mg to about 1000 mg of an amorphous form of the free acid form of the compound of Formula Ib. In some embodiments, the tablet comprises about 650 mg to about 1000 mg of an amorphous form of the free acid form of the compound of Formula Ib. In some embodiments, the tablet comprises about 800 mg to about 1000 mg of an amorphous form of the free acid form of the compound of Formula Ib. In some embodiments, the tablet comprises about 875 mg to about 925 mg of an amorphous form of the free acid form of the compound of Formula Ib. In some embodiments, the tablet comprises about 890 mg to about 910 mg of an amorphous form of the free acid form of the compound of Formula Ib. In some embodiments, the tablet comprises about 895 mg to about 905 mg of an amorphous form of the free acid form of the compound of Formula Ib. In some embodiments, the tablet comprises about 899 mg to about 901 mg of an amorphous form of the free acid form of the compound of Formula Ib. In some embodiments, the tablet comprises about 900 mg of an amorphous form of the free acid form of the compound of Formula Ib.

The tablets disclosed herein may be uncoated or coated (in which case they include an outer film coat). Although uncoated tablets may be used, it is more usual to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water-soluble material included in the film coating of the tablets may include a single polymer material, it may also be formed using a mixture of more than one polymer. The coating may be white or colored. Suitable coatings include, but are not limited to, polymeric film coatings such as those comprising polyvinyl alcohol e.g. ‘Opadry® II’ (which includes part-hydrolysed PVA, titanium dioxide, macrogol 3350 and talc, with optional colouring such as iron oxide or indigo carmine or iron oxide yellow or FD&C yellow #6). The amount of coating will generally be between about 1-8% of the uncoated tablet's weight. In some embodiments, the amount of coating will generally be between about 2-6% of the uncoated tablet's weight. In some embodiments, the amount of coating will generally be between about 3-4% of the uncoated tablet's weight.

In some embodiments, the amount of coating is 1% of the uncoated tablet's weight. In some embodiments, the amount of coating is 2% of the uncoated tablet's weight. In some embodiments, the amount of coating is 3% of the uncoated tablet's weight. In some embodiments, the amount of coating is 4% of the uncoated tablet's weight. In some embodiments, the amount of coating is 5% of the uncoated tablet's weight. In some embodiments, the amount of coating is 6% of the uncoated tablet's weight. In some embodiments, the amount of coating is 7% of the uncoated tablet's weight. In some embodiments, the amount of coating is 8% of the uncoated tablet's weight.

In some embodiments, the coating is Opadry II White 85F18422. Opadry II White 85F18422 contains: 40.00% w/w Polyvinyl Alcohol, 25.00% w/w Titanium Dioxide, 20.20% w/w Macrogol/PEG 3350, and 14.80% w/w Talc.

In some embodiments, the coating is Opadry II Grey 85F575019. Opadry II Grey 85F575019 contains: 40.00% w/w Polyvinyl Alcohol, 23.80% w/w Titanium Dioxide, 20.20% w/w Macrogol/PEG 3350, 14.80% w/w Talc, and 1.20% w/w Black Iron Oxide.

### Methods of Use

In some embodiments, the methods provided herein comprise preventing a human immunodeficiency virus (HIV) infection in the patient. In some embodiments, the patient may be at risk of contracting an HIV infection. In some embodiments, the patient has been identified as an individual who is at risk of sexual transmission of HIV. In some embodiments, the individual has been identified as a man (e.g., who has sexual intercourse with a man or a woman), transgender man, transgender woman, a woman (e.g., who has sexual intercourse with a man or a woman), as a gender non-binary individual (e.g., who has sexual intercourse with a man or a woman), and/or a sex worker.

As used herein, an “at risk” individual is an individual who is at risk of developing a condition to be treated. An individual “at risk” may or may not have detectable disease or condition, and may or may not have displayed detectable disease prior to the methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). For example, individuals at risk for AIDS are those having HIV.

In some embodiments, the individual has been identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual has been identified as having sex with partners of unknown HIV status.

In some embodiments, the individual has been identified as having sex with people who are living with HIV but not on HIV treatment and with an undetectable viral load.

In some embodiments, the individual has been identified as having sex in a geographic area where HIV is common.

In some embodiments, the individual has been identified as having sex in a geographic area of North America (e.g., United States, Canada, Mexico) where HIV is common. In some embodiments, the individual has been identified as having sex in a geographic area of the United States where HIV is common. In some embodiments, the individual has been identified as having sex in a geographic area of the Canada where HIV is common. In some embodiments, the individual has been identified as having sex in a geographic area of the Mexico where HIV is common.

In some embodiments, the individual has been identified as having sex in a geographic area of Africa where HIV is common. In some embodiments, the individual has been identified as having sex in a geographic area of South Africa where HIV is common. In some embodiments, the individual has been identified as having sex in a geographic area of Uganda where HIV is common.

In some embodiments, the individual has been identified as having sex in a geographic area of Europe where HIV is common.

In some embodiments, the individual has been identified as having sex in a geographic area of Asia (e.g., Thailand) where HIV is common. In some embodiments, the individual has been identified as having sex in a geographic area of Thailand where HIV is common.

In some embodiments, the individual has been identified as having sex in a geographic area of South America (e.g., Argentina, Brazil, Peru, and the like) where HIV is common. In some embodiments, the individual has been identified as having sex in a geographic area of Argentina where HIV is common. In some embodiments, the individual has been identified as having sex in a geographic area of Brazil where HIV is common. In some embodiments, the individual has been identified as having sex in a geographic area of Peru where HIV is common.

In some embodiments, the individual has been identified as having sex while under the influence of substances or alcohol.

In some embodiments, the individual has been identified as not using condoms consistently with partners of unknown status.

In some embodiments, the individual has been identified as an individual who injects drugs.

In some embodiments, the individual is a cisgender woman. In some embodiments, the individual is an adolescent cisgender woman. In some embodiments, the adolescent cisgender woman is about 16 to about 25 years of age, for example, about 16, about 17, about 18, about 19, about 20, about 21, about 22, about 23, about 24, about 25, or about 26 years of age.

In some embodiments, the individual is a cisgender woman identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a cisgender woman identified as having sex with partners of unknown HIV status.

In some embodiments, the individual is a cisgender woman identified as having sex with people who are living with HIV but not on HIV treatment and with an undetectable viral load.

In some embodiments, the individual is a cisgender woman identified as having sex in a geographic area where HIV is common.

In some embodiments, the individual is a cisgender woman identified as having sex in a geographic area of North America (e.g., United States, Canada, Mexico) where HIV is common. In some embodiments, the individual is a cisgender woman identified as having sex in a geographic area of the United States where HIV is common. In some embodiments, the individual is a cisgender woman identified as having sex in a geographic area of the Canada where HIV is common. In some embodiments, the individual is a cisgender woman identified as having sex in a geographic area of the Mexico where HIV is common.

In some embodiments, the individual is a cisgender woman identified as having sex in a geographic area of Africa where HIV is common. In some embodiments, the individual is a cisgender woman identified as having sex in a geographic area of South Africa where HIV is common. In some embodiments, the individual is a cisgender woman identified as having sex in a geographic area of Uganda where HIV is common.

In some embodiments, the individual is a cisgender woman identified as having sex in a geographic area of Europe where HIV is common.

In some embodiments, the individual is a cisgender woman identified as having sex in a geographic area of Asia (e.g., Thailand) where HIV is common. In some embodiments, the individual is a cisgender woman identified as having sex in a geographic area of Thailand where HIV is common.

In some embodiments, the individual is a cisgender woman identified as having sex in a geographic area of South America (e.g., Argentina, Brazil, Peru, and the like) where HIV is common. In some embodiments, the individual is a cisgender woman identified as having sex in a geographic area of Argentina where HIV is common. In some embodiments, the individual is a cisgender woman identified as having sex in a geographic area of Brazil where HIV is common. In some embodiments, the individual is a cisgender woman identified as having sex in a geographic area of Peru where HIV is common.

In some embodiments, the individual is a cisgender woman identified as having sex while under the influence of substances or alcohol.

In some embodiments, the individual is a cisgender woman identified as not using condoms consistently with partners of unknown status.

In some embodiments, the individual is a cisgender woman identified as an individual who injects drugs.

In some embodiments, the individual is a transgender woman. In some embodiments, the individual is an adolescent transgender woman. In some embodiments, the adolescent transgender woman is about 16 to about 25 years of age, for example, about 16, about 17, about 18, about 19, about 20, about 21, about 22, about 23, about 24, about 25, or about 26 years of age.

In some embodiments, the individual is a transgender woman identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a transgender woman identified as having sex with partners of unknown HIV status.

In some embodiments, the individual is a transgender woman identified as having sex with people who are living with HIV but not on HIV treatment and with an undetectable viral load.

In some embodiments, the individual is a transgender woman identified as having sex in a geographic area where HIV is common.

In some embodiments, the individual is a transgender woman identified as having sex in a geographic area of North America (e.g., United States, Canada, Mexico) where HIV is common. In some embodiments, the individual is a transgender woman identified as having sex in a geographic area of the United States where HIV is common. In some embodiments, the individual is a transgender woman identified as having sex in a geographic area of the Canada where HIV is common. In some embodiments, the individual is a transgender woman identified as having sex in a geographic area of the Mexico where HIV is common.

In some embodiments, the individual is a transgender woman identified as having sex in a geographic area of Africa where HIV is common. In some embodiments, the individual is a transgender woman identified as having sex in a geographic area of South Africa where HIV is common. In some embodiments, the individual is a transgender woman identified as having sex in a geographic area of Uganda where HIV is common.

In some embodiments, the individual is a transgender woman identified as having sex in a geographic area of Europe where HIV is common.

In some embodiments, the individual is a transgender woman identified as having sex in a geographic area of Asia (e.g., Thailand) where HIV is common. In some embodiments, the individual is a transgender woman identified as having sex in a geographic area of Thailand where HIV is common.

In some embodiments, the individual is a transgender woman identified as having sex in a geographic area of South America (e.g., Argentina, Brazil, Peru, and the like) where HIV is common. In some embodiments, the individual is a transgender woman identified as having sex in a geographic area of Argentina where HIV is common. In some embodiments, the individual is a transgender woman identified as having sex in a geographic area of Brazil where HIV is common. In some embodiments, the individual is a transgender woman identified as having sex in a geographic area of Peru where HIV is common.

In some embodiments, the individual is a transgender woman identified as having sex while under the influence of substances or alcohol.

In some embodiments, the individual is a transgender woman identified as not using condoms consistently with partners of unknown status.

In some embodiments, the individual is a transgender woman identified as an individual who injects drugs.

In some embodiments, the individual is a cisgender man. In some embodiments, the individual is an adolescent cisgender man. In some embodiments, the adolescent cisgender man is about 16 to about 25 years of age, for example, about 16, about 17, about 18, about 19, about 20, about 21, about 22, about 23, about 24, about 25, or about 26 years of age.

In some embodiments, the individual is a cisgender man identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a cisgender man identified as having sex with partners of unknown HIV status.

In some embodiments, the individual is a cisgender man identified as having sex with people who are living with HIV but not on HIV treatment and with an undetectable viral load.

In some embodiments, the individual is a cisgender man identified as having sex in a geographic area where HIV is common.

In some embodiments, the individual is a cisgender man identified as having sex in a geographic area of North America (e.g., United States, Canada, Mexico) where HIV is common. In some embodiments, the individual is a cisgender man identified as having sex in a geographic area of the United States where HIV is common. In some embodiments, the individual is a cisgender man identified as having sex in a geographic area of the Canada where HIV is common. In some embodiments, the individual is a cisgender man identified as having sex in a geographic area of the Mexico where HIV is common.

In some embodiments, the individual is a cisgender man identified as having sex in a geographic area of Africa where HIV is common. In some embodiments, the individual is a cisgender man identified as having sex in a geographic area of South Africa where HIV is common. In some embodiments, the individual is a cisgender man identified as having sex in a geographic area of Uganda where HIV is common.

In some embodiments, the individual is a cisgender man identified as having sex in a geographic area of Europe where HIV is common.

In some embodiments, the individual is a cisgender man identified as having sex in a geographic area of Asia (e.g., Thailand) where HIV is common. In some embodiments, the individual is a cisgender man identified as having sex in a geographic area of Thailand where HIV is common.

In some embodiments, the individual is a cisgender man identified as having sex in a geographic area of South America (e.g., Argentina, Brazil, Peru, and the like) where HIV is common. In some embodiments, the individual is a cisgender man identified as having sex in a geographic area of Argentina where HIV is common. In some embodiments, the individual is a cisgender man identified as having sex in a geographic area of Brazil where HIV is common. In some embodiments, the individual is a cisgender man identified as having sex in a geographic area of Peru where HIV is common.

In some embodiments, the individual is a cisgender man identified as having sex while under the influence of substances or alcohol.

In some embodiments, the individual is a cisgender man identified as not using condoms consistently with partners of unknown status.

In some embodiments, the individual is a cisgender man identified as an individual who injects drugs.

In some embodiments, the individual is a transgender man. In some embodiments, the individual is an adolescent transgender man. In some embodiments, the adolescent transgender man is about 16 to about 25 years of age, for example, about 16, about 17, about 18, about 19, about 20, about 21, about 22, about 23, about 24, about 25, or about 26 years of age.

In some embodiments, the individual is a transgender man identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
   an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a transgender man identified as having sex with partners of unknown HIV status.

In some embodiments, the individual is a transgender man identified as having sex with people who are living with HIV but not on HIV treatment and with an undetectable viral load.

In some embodiments, the individual is a transgender man identified as having sex in a geographic area where HIV is common.

In some embodiments, the individual is a transgender man identified as having sex in a geographic area of North America (e.g., United States, Canada, Mexico) where HIV is common. In some embodiments, the individual is a transgender man identified as having sex in a geographic area of the United States where HIV is common. In some embodiments, the individual is a transgender man identified as having sex in a geographic area of the Canada where HIV is common. In some embodiments, the individual is a transgender man identified as having sex in a geographic area of the Mexico where HIV is common.

In some embodiments, the individual is a transgender man identified as having sex in a geographic area of Africa where HIV is common. In some embodiments, the individual is a transgender man identified as having sex in a geographic area of South Africa where HIV is common. In some embodiments, the individual is a transgender man identified as having sex in a geographic area of Uganda where HIV is common.

In some embodiments, the individual is a transgender man identified as having sex in a geographic area of Europe where HIV is common.

In some embodiments, the individual is a transgender man identified as having sex in a geographic area of Asia (e.g., Thailand) where HIV is common. In some embodiments, the individual is a transgender man identified as having sex in a geographic area of Thailand where HIV is common.

In some embodiments, the individual is a transgender man identified as having sex in a geographic area of South America (e.g., Argentina, Brazil, Peru, and the like) where HIV is common. In some embodiments, the individual is a transgender man identified as having sex in a geographic area of Argentina where HIV is common. In some embodiments, the individual is a transgender man identified as having sex in a geographic area of Brazil where HIV is common. In some embodiments, the individual is a transgender man identified as having sex in a geographic area of Peru where HIV is common.

In some embodiments, the individual is a transgender man identified as having sex while under the influence of substances or alcohol.

In some embodiments, the individual is a transgender man identified as not using condoms consistently with partners of unknown status.

In some embodiments, the individual is a transgender man identified as an individual who injects drugs.

In some embodiments, the individual is a gender non-binary individual. In some embodiments, the individual is an adolescent gender non-binary individual. In some embodiments, the adolescent gender non-binary individual is about 16 to about 25 years of age, for example, about 16, about 17, about 18, about 19, about 20, about 21, about 22, about 23, about 24, about 25, or about 26 years of age.

In some embodiments, the individual is a gender non-binary individual identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a gender non-binary individual identified as having sex with partners of unknown HIV status.

In some embodiments, the individual is a gender non-binary individual identified as having sex with people who are living with HIV but not on HIV treatment and with an undetectable viral load.

In some embodiments, the individual is a gender non-binary individual identified as having sex in a geographic area where HIV is common.

In some embodiments, the individual is a gender non-binary individual identified as having sex in a geographic area of North America (e.g., United States, Canada, Mexico) where HIV is common. In some embodiments, the individual is a gender non-binary individual identified as having sex in a geographic area of the United States where HIV is common. In some embodiments, the individual is a gender non-binary individual identified as having sex in a geographic area of the Canada where HIV is common. In some embodiments, the individual is a gender non-binary individual identified as having sex in a geographic area of the Mexico where HIV is common.

In some embodiments, the individual is a gender non-binary individual identified as having sex in a geographic area of Africa where HIV is common. In some embodiments, the individual is a gender non-binary individual identified as having sex in a geographic area of South Africa where HIV is common. In some embodiments, the individual is a gender non-binary individual identified as having sex in a geographic area of Uganda where HIV is common.

In some embodiments, the individual is a gender non-binary individual identified as having sex in a geographic area of Europe where HIV is common.

In some embodiments, the individual is a gender non-binary individual identified as having sex in a geographic area of Asia (e.g., Thailand) where HIV is common. In some embodiments, the individual is a gender non-binary individual identified as having sex in a geographic area of Thailand where HIV is common.

In some embodiments, the individual is a gender non-binary individual identified as having sex in a geographic area of South America (e.g., Argentina, Brazil, Peru, and the like) where HIV is common. In some embodiments, the individual is a gender non-binary individual identified as having sex in a geographic area of Argentina where HIV is common. In some embodiments, the individual is a gender non-binary individual identified as having sex in a geographic area of Brazil where HIV is common. In some embodiments, the individual is a gender non-binary individual identified as having sex in a geographic area of Peru where HIV is common.

In some embodiments, the individual is a gender non-binary individual identified as having sex while under the influence of substances or alcohol.

In some embodiments, the individual is a gender non-binary individual identified as not using condoms consistently with partners of unknown status.

In some embodiments, the individual is a gender non-binary individual identified as an individual who injects drugs.

In some embodiments, the individual has been identified as:
having anal sex with at least two different sexual partners and no consistent condom use over the last 6 months; and/or
having history of sexually transmitted diseases (STDs) during the last 12 months (e.g., syphilis, gonorrhea, chlamydiae, HBV or HCV infection); and/or
using psycho-active drugs during sexual intercourses (e.g., cocaine, gammahydroxybutyric acid (GHB), methylenedioxymethamphetamine (MDMA), mephedrone); and/or
having sexual intercourse with one or more partners originating from a region with high prevalence of HIV infection (>1%) (e.g., South America, Sub-Saharan Africa, South-East Asia, Eastern Europe, French Guyana) and no consistent condom use; and/or
a sex worker; and/or
having a sexual partner who is an intravenous drug user sharing injection material; and/or
having an HIV-infected sexual partner with a detectable plasma viral load (e.g., >50 copies (cp)/milliliter (mL)); and/or
a cisgender man;
a transgender man;
a cisgender woman;
a transgender woman;
a gender non-binary individual; and/or
a person who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the methods provided herein comprise administering to the patient a prophylactically effective amount of the compound of Formula Ia or Ib, or a pharmaceutically acceptable salt thereof, and a compound of Formula II, or a pharmaceutically acceptable salt thereof, for pre-exposure prophylaxis (PrEP) to prevent sexually acquired HIV-1 in adults and adolescents. In some embodiments, the methods provided herein comprise administering to the patient a prophylactically effective amount of the compound of Formula Ia or Ib, or a pharmaceutically acceptable salt thereof, and a compound of Formula II, or a pharmaceutically acceptable salt thereof, for pre-exposure prophylaxis (PrEP) to prevent sexually acquired HIV-1 in adults and adolescents weighing at least 35 kg. In some embodiments, the patient has obtained a negative HIV-1 test prior to administration of the compound of Formula Ia or Ib, or a pharmaceutically acceptable salt thereof, and a compound of Formula II, or a pharmaceutically acceptable salt thereof.

In some embodiments, the patient is HIV-negative. In some embodiments, the HIV is HIV-1. In some embodiments, the HIV is HIV-2. In some embodiments, the HIV is HIV-1 and HIV-2.

As used herein, “HIV” or “Human Immunodeficiency Virus” refers to HIV-1 and/or to HIV-2.

The term “patient” is meant to refer to a human who is in need of preventative treatment for a viral infection, such as HIV infection.

As used herein, the terms “prevention” or “preventing” refer to the administration of a compound, or a pharmaceutically acceptable salt or free acid form thereof, or composition comprising the compound, pharmaceutically acceptable salt, or free acid form thereof according to the present disclosure pre- or post-exposure of the patient to the virus but before the appearance of symptoms of the disease, and/or prior to the detection of the virus in the blood. The terms also refer to prevention of the appearance of symptoms of the disease and/or to prevent the virus from reaching detectable levels in the blood. The terms include both pre-exposure prophylaxis (PrEP), as well as post-exposure prophylaxis (PEP) and event driven or “on demand” prophylaxis. The terms also refer to prevention of perinatal transmission of HIV from mother to baby by administration of a compound, pharmaceutically acceptable salt thereof, or composition comprising the compound or the pharmaceutically acceptable salt thereof according to the present disclosure to the mother before giving birth and to the child within the first days of life. The term also refers to prevention of transmission of HIV through blood transfusion.

As used herein, the term “period of exposure” refers to a period of time, ranging from a single event or to multiple events over an extended period of time, in which a patient is exposed to HIV. For example, a patient who engages in one sexual intercourse event with a partner who is HIV-positive has a period of exposure that is limited to the time and duration of that one sexual intercourse event with that partner. As another example, a patient who has sexual intercourse with a partner who is HIV-positive on multiple occasions over an extended period of time (e.g., days, weeks, months, or years) has a period of exposure that ranges from the first instance to the last instance of sexual intercourse with that partner.

In some embodiments, the methods disclosed herein may comprise event driven administration of a composition provided herein (e.g., a composition comprising a compound of Formula Ia or Ib, or a pharmaceutically acceptable salt or free acid form thereof), to the patient. As used herein, the terms “event driven” or “event driven administration” refer to administration of a composition provided herein, (1) prior to an event (e.g., 2 hours, 1 day, 2 days, 5 days, 7 days, 10 days, 14 days, 28 days (i.e., one month), or more days prior to the event) that would expose the patient to HIV (or that would otherwise increase the patient's risk of acquiring HIV); and/or (2) during an event (or more than one recurring event) that would expose the patient to HIV (or that would otherwise increase the patient's risk of acquiring HIV); and/or (3) after an event (or after the final event in a series of recurring events) that would expose the patient to HIV (or that would otherwise increase the patient's risk of acquiring HIV). In some embodiments, the event driven administration is performed pre-exposure of the patient to the HIV. In some embodiments, the event driven administration is performed during exposure of the patient to the HIV. In some embodiments, the event driven administration is performed post-exposure of the patient to the HIV.

In some embodiments, the event driven administration is performed pre-exposure of the patient to the HIV and during exposure of the patient to the HIV.

In some embodiments, the event driven administration is performed pre-exposure of the patient to the HIV and post-exposure of the patient to the HIV.

In some embodiments, the event driven administration is performed during exposure of the patient to the HIV and post-exposure of the patient to the HIV.

In certain embodiments, the methods disclosed herein involve administration prior to and/or after an event that would expose the patient to HIV or that would otherwise increase the patient's risk of acquiring HIV, e.g., as pre-exposure prophylaxis (PrEP) and/or as post-exposure prophylaxis (PEP). Examples of events that could increase a patient's risk of acquiring HIV include, without limitation, no condom use during anal intercourse with an HIV positive partner or a partner of unknown HIV status; anal intercourse with more than 3 sexual partners; exchange of money, gifts, shelter or drugs for anal sex; sex with male partner and diagnosis of sexually transmitted infection; and no consistent use of condoms with a sexual partner known to be HIV positive. In some embodiments, the methods disclosed herein comprise pre-exposure prophylaxis (PrEP). In some embodiments, methods disclosed herein comprise post-exposure prophylaxis (PEP). In some embodiments, the methods disclosed herein comprise pre-exposure prophylaxis (PrEP) and post-exposure prophylaxis (PEP).

In some embodiments, a composition provided herein is administered before exposure of the patient to the HIV.

In some embodiments, a composition provided herein is administered during the period of exposure of the patient to the HIV.

In some embodiments, a composition provided herein is administered after final exposure of the patient to the HIV.

In some embodiments, a composition provided herein is administered before and during exposure of the patient to the HIV.

In some embodiments, a composition provided herein is administered before and after exposure of the patient to the HIV.

In some embodiments, a composition provided herein is administered during and after exposure of the patient to the HIV.

In some embodiments, a composition provided herein is administered before, during, and after exposure of the patient to the HIV.

In some embodiments, the dose of the composition administered during each period (i.e., before, during, and after exposure) may be different, i.e, independently selected from any of the doses disclosed herein.

In certain embodiments, e.g., when administered as PrEP, a composition provided herein is administered 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity, prior to sexual intercourse or other exposure to the HIV). In some embodiments, a composition provided herein is administered within 14 days, 13 days, 12 days, 11 days, 10 days, 9 days, 8 days, 7 days, 6 days, 5 days, 4 days, 3 days, 2 days, or 1 day prior to an event that would increase the patient's risk of acquiring HIV (e.g., prior to sexual intercourse or other exposure to the HIV). In some embodiments, a composition provided herein is administered within 72 hours, 60 hours, 48 hours, 24 hours, 12 hours, 9 hours, 6 hours, 4 hours, 3 hours, 2 hours, or 1 hour prior to an event that would increase the patient's risk of acquiring HIV (e.g., prior to sexual intercourse or other exposure to the HIV). In certain embodiments, when a composition provided herein is administered prior to an event that would increase the patient's risk of acquiring HIV, it is administered daily prior to the event (e.g., sexual activity). In certain embodiments, when a composition provided herein is administered prior to an event that would increase the patient's risk of acquiring HIV, it is administered one to three times prior to the event. In certain embodiments, when a composition provided herein is administered prior to an event that would increase the patient's risk of acquiring HIV, it is administered one time (i.e., once) prior to the event.

In some embodiments, a composition provided herein is administered once from about 31 days to about one day before exposure of the patient to the HIV. In some embodiments, a composition provided herein is administered once from about 30 days to about one day before exposure of the patient to the HIV. In some embodiments, a composition provided herein is administered from about 14 days to about one day before exposure of the patient to the HIV. In some embodiments, a composition provided herein is administered once from about 14 days to about one day before exposure of the patient to the HIV.

In some embodiments, a composition provided herein is administered from about 10 days to about 5 days before exposure of the patient to the HIV. In some embodiments, a composition provided herein is administered once from about 10 days to about 5 days before exposure of the patient to the HIV.

In some embodiments, a composition provided herein is administered from about 8 days to about 6 days before exposure of the patient to the HIV. In some embodiments, a composition provided herein is administered once from about 8 days to about 6 days before exposure of the patient to the HIV.

In some embodiments, a composition provided herein is administered about 7 days before exposure of the patient to the HIV. In some embodiments, a composition provided herein is administered once about 7 days before exposure of the patient to the HIV.

In some embodiments, a composition provided herein is administered from about 72 hours to about 1 hour before exposure of the patient to the HIV. In some embodiments, a composition provided herein is administered once from about 72 hours to about 1 hour before exposure of the patient to the HIV.

In some embodiments of the methods provided herein, the pre-exposure prophylaxis (PrEP) comprises continuous PrEP.

In certain embodiments where a composition provided herein is administered before exposure of the patient to the HIV, the methods disclosed herein further comprise administering one or more additional doses of the compound of Formula Ia or Ib, or a pharmaceutically acceptable salt or free acid form thereof, during, and/or after exposure of the patient to the HIV.

In some embodiments, e.g., when administered as part of a PrEP regimen or as part of a PEP regimen, a composition provided herein is administered during the period of exposure of the patient to the HIV. In certain embodiments wherein a composition provided herein is administered before HIV exposure, the composition is administered about every 7 days, about every 14 days, about every 21 days, about every 28 days, about every 35 days, about every 42 days, or about every 6 months, or about every 12 months (e.g., as a single dose) during the time of HIV exposure (e.g., during the time period of sexual activity with a sexual partner known to be HIV positive). In some embodiments, a composition provided herein is administered once about every 7 days, about every 14 days, about every 21 days, about every 28 days, about every 35 days, about every 42 days, about every 6 months, or about every 12 months during the period of exposure of the patient to the HIV.

In some embodiments, a composition provided herein administered prior to exposure to the HIV is at a different dose than a composition (e.g., a composition comprising a compound of Formula Ia or Ib, or a pharmaceutically acceptable salt or free acid form thereof) administered during and/or after exposure to the HIV. For example, in some embodiments, the dose of the compound of Formula Ia or Ib, or a pharmaceutically acceptable salt or free acid form thereof, is increased, e.g., as a double dose, as a triple dose, and the like as compared to an earlier administered dose (e.g., a dose prior to exposure to the HIV). In some embodiments, the increased dose of the compound of Formula Ia or Ib, or a pharmaceutically acceptable salt or free acid form thereof, is a double dose. In some embodiments, the dose of the compound of Formula Ia or Ib, or a pharmaceutically acceptable salt or free acid form thereof, is decreased, e.g., a half dose as compared to an earlier administered dose (e.g., a dose prior to exposure to the HIV).

In some embodiments, a composition provided herein (e.g., a composition comprising a compound of Formula Ia or Ib, or a pharmaceutically acceptable salt or free acid form thereof) is administered as a single dose from about 1 hour to about 10 days before exposure of the patient to the HIV.

Additional examples of PrEP and/or PEP can be found, for example, at the clinical trial summary titled “On Demand Antiretroviral Pre-exposure Prophylaxis for HIV Infection in Men Who Have Sex With Men” (Clinical Trial #NCT01473472); the clinical trial summary titled “Prevention of HIV in Île-de-France” (Clinical Trials #NCT03113123), and at Molina et al, N. Engl. J. Med. 2015, 353:2237-2246, the disclosure of each of which is incorporated herein by reference in its entirety.

In some embodiments, e.g., when administered as part of a PrEP regimen or as part of a PEP regimen, a composition provided herein is administered 1 hour to 10 days, 1 hour to 7 days, 1 hour to 5 days, 1 to 72 hours, 1 to 48 hours, 1 to 36 hours, 1 to 24 hours, or 1 to 12 hours following an event that would increase the patient's risk of acquiring HIV (e.g., following sexual intercourse or other exposure to the HIV).

In certain embodiments, e.g., when administered as PEP, a composition provided herein is administered for 7 days, 14 days, 21 days, 28 days, 30 days, or 45 days following an event that would increase the patient's risk of acquiring HIV (e.g., following sexual intercourse or other exposure to the HIV). In certain embodiments, e.g., when administered as PEP, a composition provided herein is administered for 30 days following an event that would increase the patient's risk of acquiring HIV (e.g., following sexual intercourse or other exposure to the HIV). In certain embodiments, a composition provided herein is administered less than 1 hour, 2 hours, 3 hours, 4 hours, 5 hours, 6 hours, 7 hours, 8 hours, 9 hours, 12 hours, 18 hours, 24 hours, 36 hours, or 48 hours following an event that would increase the patient's risk of acquiring HIV (e.g., following sexual intercourse or other exposure to the HIV virus). In certain embodiments, a composition provided herein is administered for 1 day, 2 days, 3 days, 4 days, or 5 days following an event that would increase the patient's risk of acquiring HIV (e.g., following sexual intercourse or other exposure to the HIV). In certain embodiments, when a composition provided herein is administered following an event that would increase the patient's risk of acquiring HIV, it is administered daily following the event. In certain embodiments, when a composition provided herein is administered following an event that would increase the patient's risk of acquiring HIV, it is administered one to three times following the event. In certain embodiments, when a composition provided herein is administered following an event that would increase the patient's risk of acquiring HIV, it is administered once following the event.

In certain embodiments, e.g., when administered as PEP, a composition provided herein is administered once about every week, once about every month, once about every 2 months, once about every 3 months, once about every 4 months, once about every 5 months, once about every 6 months, or once about every 12 months following an event that would increase the patient's risk of acquiring HIV (e.g., following sexual intercourse or other exposure to the HIV). In certain embodiments, e.g., when administered as PEP, a composition provided herein is administered once about every week following an event that would increase the patient's risk of acquiring HIV (e.g., following sexual intercourse or other exposure to the HIV). In certain embodiments, e.g., when administered as PEP, a composition provided herein is administered once about every month following an event that would increase the patient's risk of acquiring HIV (e.g., following sexual intercourse or other exposure to the HIV). In certain embodiments, e.g., when administered as PEP, a composition provided herein is administered once about every 2 months following an event that would increase the patient's risk of acquiring HIV (e.g., following sexual intercourse or other exposure to the HIV). In certain embodiments, e.g., when administered as PEP, a composition provided herein is administered once about every 3 months following an event that would increase the patient's risk of acquiring HIV (e.g., following sexual intercourse or other exposure to the HIV). In certain embodiments, e.g., when administered as PEP, a composition provided herein is administered once about every 4 months following an event that would increase the patient's risk of acquiring HIV (e.g., following sexual intercourse or other exposure to the HIV). In certain embodiments, e.g., when administered as PEP, a composition provided herein is administered once about every 5 months following an event that would increase the patient's risk of acquiring HIV (e.g., following sexual intercourse or other exposure to the HIV). In certain embodiments, e.g., when administered as PEP, a composition provided herein is administered once about every 6 months following an event that would increase the patient's risk of acquiring HIV (e.g., following sexual intercourse or other exposure to the HIV). In certain embodiments, e.g., when administered as PEP, a composition provided herein is administered once about every 12 months following an event that would increase the patient's risk of acquiring HIV (e.g., following sexual intercourse or other exposure to the HIV).

In certain embodiments, e.g., when administered as PEP, a composition provided herein is administered for one month, two months, three months, four months, five months, six months, or twelve months following an event that would increase the patient's risk of acquiring HIV (e.g., following sexual intercourse or other exposure to the HIV).

In certain embodiments, when a composition provided herein is administered following an event that would increase the patient's risk of acquiring HIV, it is administered one to fifty times following the event. In certain embodiments, when a composition provided herein is administered following an event that would increase the patient's risk of acquiring HIV, it is administered one to forty times following the event. In certain embodiments, when a composition provided herein is administered following an event that would increase the patient's risk of acquiring HIV, it is administered one to thirty times following the event. In certain embodiments, when a composition provided herein is administered following an event that would increase the patient's risk of acquiring HIV, it is administered one to twenty times following the event. In certain embodiments, when a composition provided herein is administered following an event that would increase the patient's risk of acquiring HIV, it is administered one to fifteen times following the event. In certain embodiments, when a composition provided herein is administered following an event that would increase the patient's risk of acquiring HIV, it is administered one to ten times following the event. In certain embodiments, when a composition provided herein is administered following an event that would increase the patient's risk of acquiring HIV, it is administered one to five times following the event.

In some embodiments, a composition provided herein is administered during exposure of the patient to the HIV (e.g., during a period of sexual activity with a sexual partner known to be HIV positive).

In some embodiments, a composition provided herein is administered after exposure (e.g., after final exposure) of the patient to the HIV (e.g., after a period of sexual activity with a sexual partner known to be HIV positive). In some embodiments, a composition provided herein is administered from about 1 hour to about 14 days after exposure (e.g., after final exposure) of the patient to the HIV. In some embodiments, a composition provided herein is administered once from about 1 hour to about 14 days after exposure (e.g., after final exposure) of the patient to the HIV. In some embodiments, a composition provided herein is administered from about 1 hour to about 7 days after exposure (e.g., after final exposure) of the patient to the HIV. In some embodiments, a composition provided herein is administered once from about 1 hour to about 7 days after exposure (e.g., after final exposure) of the patient to the HIV. In some embodiments, a composition provided herein is administered from about 1 hour to about 72 hours after exposure (e.g., after final exposure) of the patient to the HIV. In some embodiments, a composition provided herein is administered once from about 1 hour to about 72 hours after exposure (e.g., after final exposure) of the patient to the HIV. In some embodiments, a composition provided herein is administered from about 1 hour to about 24 hours after exposure (e.g., after final exposure) of the patient to the HIV. In some embodiments, a composition provided herein is administered once from about 1 hour to about 24 hours after exposure (e.g., after final exposure) of the patient to the HIV. In some embodiments, a composition provided herein is administered from about 24 hours to about 72 hours after exposure (e.g., after final exposure) of the patient to the HIV. In some embodiments, a composition provided herein is administered once from about 24 hours to about 72 hours after exposure (e.g., after final exposure) of the patient to the HIV.

In some embodiments, e.g., when administered as PrEP, a composition provided herein is administered prior to an event that would increase the patient's risk of acquiring HIV (e.g., prior to sexual activity), and following the event. For example, in certain embodiments, when administered as PrEP, a composition provided herein is administered 1 to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity) and 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 36 hours, 1 hour to 24 hours, or 1 hour to 12 hours following the event. For example, in some embodiments, one or more (e.g., one, two, or three) dosages of a composition provided herein are administered one to ten days (e.g., seven days) prior to an event that would increase the patient's risk of acquiring HIV (e.g., prior to sexual intercourse) and once during a period of one to ten days following the event. In some embodiments, a composition provided herein is administered once per week, twice per week, three times per week, four times per week, or five times per week and one or more times (e.g., one, two, or three times) beginning 1 to 48 hours following an event that would increase the patient's risk of acquiring HIV (e.g., following sexual intercourse).

Also provided herein is a method of reducing the risk of acquiring HIV in a patient, comprising administering to the patient a composition provided herein (e.g., a composition comprising a compound of Formula Ia or Ib, or a pharmaceutically acceptable salt or free acid form thereof).

In some embodiments, methods for reducing the risk of acquiring HIV (e.g., HIV-1 and/or HIV-2) comprise administration of a composition provided herein to a patient in combination with safer sexual intercourse practices. In certain embodiments, methods for reducing the risk of acquiring HIV (e.g., HIV-1 and/or HIV-2) comprise administration of a composition provided herein to a patient at risk of acquiring HIV. Examples of patients at high risk for acquiring HIV include, without limitation, a patient who is at risk of sexual transmission of HIV.

In some embodiments, the reduction in risk of acquiring HIV is at least about 40%, 50%, 60%, 70%, 80%, 90%, or 95% (compared to a patient having not been administered the compound of Formula Ia or Ib, or a pharmaceutically acceptable salt thereof according to any of the methods provided herein). In some embodiments, the reduction in risk of acquiring HIV is about 80%, 85%, or 90%. In some embodiments, the reduction in risk of acquiring HIV is at least about 75%. In some embodiments, the reduction in risk of acquiring HIV is at least about 80%. In some embodiments, the reduction in risk of acquiring HIV is at least about 85%. In some embodiments, the reduction in risk of acquiring HIV is at least about 90%.

In some embodiments, a composition provided herein (e.g., a tablet comprising a compound of Formula Ia or Ib, or a pharmaceutically acceptable salt or free acid form thereof) is suitable for oral administration. In some embodiments, a composition provided herein (e.g., a tablet comprising a compound of Formula Ia or Ib, or a pharmaceutically acceptable salt or free acid form thereof) is administered orally.

In some embodiments, a composition provided herein (e.g., a tablet comprising a compound of Formula Ia or Ib, or a pharmaceutically acceptable salt or free acid form thereof) is administered once every five to ten days, for example, once every five days, six days, seven days, eight days, nine days, or ten days. In some embodiments, a composition provided herein (e.g., a tablet comprising a compound of Formula Ia or Ib, or a pharmaceutically acceptable salt or free acid form thereof) is administered once every six to eight days. In some embodiments, a composition provided herein (e.g., a tablet comprising a compound of Formula Ia or Ib, or a pharmaceutically acceptable salt or free acid form thereof) is administered once every seven days (i.e., once-weekly).

In some embodiments, a composition provided herein (e.g., a tablet comprising a compound of Formula Ia or Ib, or a pharmaceutically acceptable salt or free acid form thereof) is orally administered once every five to ten days, for example, once every five days, six days, seven days, eight days, nine days, or ten days. In some embodiments, a composition provided herein (e.g., a tablet comprising a compound of Formula Ia or Ib, or a pharmaceutically acceptable salt or free acid form thereof) is orally administered once every six to eight days. In some embodiments, a composition provided herein (e.g., a tablet comprising a compound of Formula Ia or Ib, or a pharmaceutically acceptable salt or free acid form thereof) is orally administered once every seven days (i.e., once-weekly).

In some embodiments, the method comprises administering to the patient a tablet comprising or consists essentially of:
about 30 w/w % to about 80 w/w % of the compound of Formula Ia or Ib, or a pharmaceutically acceptable salt thereof;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

In some embodiments, the method comprises administering to the patient a tablet comprising or consisting essentially of:
about 50 w/w % to about 70 w/w % of the compound of Formula Ia or Ib, or a pharmaceutically acceptable salt thereof;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the method comprises administering said tablet once every month or once every week.

In some embodiments, the method comprises administering to the patient a tablet comprising or consisting essentially of:
about 55 w/w % to about 65 w/w % of the compound of Formula Ia or Ib, or a pharmaceutically acceptable salt thereof;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the method comprises administering said tablet once every month or once every week.

In some embodiments, the method comprises administering to the patient a tablet comprising or consisting essentially of:
about 60 w/w % of the compound of Formula Ia or Ib, or a pharmaceutically acceptable salt thereof;
about 28.5 w/w % of microcrystalline cellulose;
about 10 w/w % of crospovidone; and
about 1.5 w/w % of magnesium stearate.

In some embodiments, the method comprises administering to the patient a tablet comprising or consisting essentially of:
about 60 w/w % of the compound of Formula Ia or Ib, or a pharmaceutically acceptable salt thereof;
about 28 w/w % of microcrystalline cellulose;
about 10 w/w % of crospovidone; and
about 2 w/w % of magnesium stearate.

In some embodiments, the method comprises administering said tablet once every month or once every week.

In some embodiments, the method comprises administering to the patient a tablet comprising or consisting essentially of:
about 30 w/w % to about 80 w/w % of the free acid form of the compound of Formula Ia or Ib;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

In some embodiments, the method comprises administering to the patient a tablet comprising or consisting essentially of:
about 50 w/w % to about 70 w/w % of the free acid form of the compound of Formula Ia or Ib;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the method comprises administering to the patient a tablet comprising or consisting essentially of:
about 55 w/w % to about 65 w/w % of the free acid form of the compound of Formula Ia or Ib;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the method comprises administering to the patient a tablet comprising or consisting essentially of:
about 60 w/w % of the free acid form of the compound of Formula Ia or Ib;
about 28.5 w/w % of microcrystalline cellulose;
about 10 w/w % of crospovidone; and
about 1.5 w/w % of magnesium stearate.

In some embodiments, the method comprises administering to the patient a tablet comprising or consisting essentially of:
about 60 w/w % of the free acid form of the compound of Formula Ia or Ib;
about 28 w/w % of microcrystalline cellulose;
about 10 w/w % of crospovidone; and
about 2 w/w % of magnesium stearate.

In some embodiments, the method comprises administering to the patient a tablet comprising or consisting essentially of:
about 30 w/w % to about 80 w/w % of an amorphous form of the free acid form of the compound of Formula Ia or Ib;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

In some embodiments, the method comprises administering to the patient a tablet comprising or consisting essentially of:
about 50 w/w % to about 70 w/w % of an amorphous form of the free acid form of the compound of Formula Ia or Ib;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the method comprises administering to the patient a tablet comprising or consisting essentially of:
about 55 w/w % to about 65 w/w % of an amorphous form of the free acid form of the compound of Formula Ia or Ib;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the method comprises administering to the patient a tablet comprising or consisting essentially of:
about 60 w/w % of an amorphous form of the free acid form of the compound of Formula Ia or Ib;
about 28.5 w/w % of microcrystalline cellulose;
about 10 w/w % of crospovidone; and
about 1.5 w/w % of magnesium stearate.

In some embodiments, the method comprises administering to the patient a tablet comprising or consisting essentially of:
about 60 w/w % of an amorphous form of the free acid form of the compound of Formula Ia or Ib;
about 28 w/w % of microcrystalline cellulose;
about 10 w/w % of crospovidone; and
about 2 w/w % of magnesium stearate.

In some embodiments, the method comprises administering to the patient a tablet comprising or consisting essentially of:
about 30 w/w % to about 80 w/w % of the compound of Formula Ia, or a pharmaceutically acceptable salt thereof;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

In some embodiments, the method comprises administering to the patient a tablet comprising or consisting essentially of:
about 50 w/w % to about 70 w/w % of the compound of Formula Ia, or a pharmaceutically acceptable salt thereof;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the method comprises administering to the patient a tablet comprising or consisting essentially of:
about 55 w/w % to about 65 w/w % of the compound of Formula Ia, or a pharmaceutically acceptable salt thereof;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the method comprises administering to the patient a tablet comprising or consisting essentially of:
about 60 w/w % of the compound of Formula Ia, or a pharmaceutically acceptable salt thereof;
about 28.5 w/w % of microcrystalline cellulose;
about 10 w/w % of crospovidone; and
about 1.5 w/w % of magnesium stearate.

In some embodiments, the method comprises administering to the patient a tablet comprising or consisting essentially of:
about 60 w/w % of the compound of Formula Ia, or a pharmaceutically acceptable salt thereof;
about 28 w/w % of microcrystalline cellulose;
about 10 w/w % of crospovidone; and
about 2 w/w % of magnesium stearate.

In some embodiments, the method comprises administering to the patient a tablet comprising or consisting essentially of:
about 30 w/w % to about 80 w/w % of the free acid form of the compound of Formula Ia;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

In some embodiments, the method comprises administering to the patient a tablet comprising or consisting essentially of:
about 50 w/w % to about 70 w/w % of the free acid form of the compound of Formula Ia;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the method comprises administering to the patient a tablet comprising or consisting essentially of:
about 55 w/w % to about 65 w/w % of the free acid form of the compound of Formula Ia;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the method comprises administering to the patient a tablet comprising or consisting essentially of:
about 60 w/w % of the free acid form of the compound of Formula Ia;
about 28.5 w/w % of microcrystalline cellulose;
about 10 w/w % of crospovidone; and
about 1.5 w/w % of magnesium stearate.

In some embodiments, the method comprises administering to the patient a tablet comprising or consisting essentially of:
about 60 w/w % of the free acid form of the compound of Formula Ia;
about 28 w/w % of microcrystalline cellulose;
about 10 w/w % of crospovidone; and
about 2 w/w % of magnesium stearate.

In some embodiments, the method comprises administering to the patient a tablet comprising or consisting essentially of:
about 30 w/w % to about 80 w/w % of an amorphous form of the free acid form of the compound of Formula Ia;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

In some embodiments, the method comprises administering to the patient a tablet comprising or consisting essentially of:
about 50 w/w % to about 70 w/w % of an amorphous form of the free acid form of the compound of Formula Ia;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the method comprises administering to the patient a tablet comprising or consisting essentially of:
about 55 w/w % to about 65 w/w % of an amorphous form of the free acid form of the compound of Formula Ia;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the method comprises administering to the patient a tablet comprising or consisting essentially of:
about 60 w/w % of an amorphous form of the free acid form of the compound of Formula Ia;
about 28.5 w/w % of microcrystalline cellulose;
about 10 w/w % of crospovidone; and about 1.5 w/w % of magnesium stearate.

In some embodiments, the method comprises administering to the patient a tablet comprising or consisting essentially of:
about 60 w/w % of an amorphous form of the free acid form of the compound of Formula Ia;
about 28 w/w % of microcrystalline cellulose;
about 10 w/w % of crospovidone; and
about 2 w/w % of magnesium stearate.

In some embodiments, the method comprises administering to the patient a tablet comprising or consisting essentially of:
about 30 w/w % to about 80 w/w % of the compound of Formula Ib, or a pharmaceutically acceptable salt thereof;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

In some embodiments, the method comprises administering to the patient a tablet comprising or consisting essentially of:
about 50 w/w % to about 70 w/w % of the compound of Formula Ib, or a pharmaceutically acceptable salt thereof;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the method comprises administering said tablet once every month or once every week.

In some embodiments, the method comprises administering to the patient a tablet comprising or consisting essentially of:
about 55 w/w % to about 65 w/w % of the compound of Formula Ib, or a pharmaceutically acceptable salt thereof;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the method comprises administering said tablet once every month or once every week.

In some embodiments, the method comprises administering to the patient a tablet comprising or consisting essentially of:
about 60 w/w % of the compound of Formula Ib, or a pharmaceutically acceptable salt thereof;
about 28.5 w/w % of microcrystalline cellulose;
about 10 w/w % of crospovidone; and
about 1.5 w/w % of magnesium stearate.

In some embodiments, the method comprises administering to the patient a tablet comprising or consisting essentially of:
about 60 w/w % of the compound of Formula Ib, or a pharmaceutically acceptable salt thereof;
about 28 w/w % of microcrystalline cellulose;
about 10 w/w % of crospovidone; and
about 2 w/w % of magnesium stearate.

In some embodiments, the method comprises administering said tablet once every month or once every week.

In some embodiments, the method comprises administering to the patient a tablet comprising or consisting essentially of:
about 30 w/w % to about 80 w/w % of the free acid form of the compound of Formula Ib;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

In some embodiments, the method comprises administering said tablet once every month or once every week.

In some embodiments, the method comprises administering to the patient a tablet comprising or consisting essentially of:
about 50 w/w % to about 70 w/w % of the free acid form of the compound of Formula Ib;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the method comprises administering said tablet once every month or once every week.

In some embodiments, the method comprises administering to the patient a tablet comprising or consisting essentially of:
about 55 w/w % to about 65 w/w % of the free acid form of the compound of Formula Ib;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the method comprises administering said tablet once every month or once every week.

In some embodiments, the method comprises administering to the patient a tablet comprising or consisting essentially of:
about 60 w/w % of the free acid form of the compound of Formula Ib;
about 28.5 w/w % of microcrystalline cellulose;
about 10 w/w % of crospovidone; and
about 1.5 w/w % of magnesium stearate.

In some embodiments, the method comprises administering to the patient a tablet comprising or consisting essentially of:
about 60 w/w % of the free acid form of the compound of Formula Ib;
about 28 w/w % of microcrystalline cellulose;
about 10 w/w % of crospovidone; and
about 2 w/w % of magnesium stearate.

In some embodiments, the method comprises administering said tablet once every month or once every week.

In some embodiments, the method comprises administering to the patient a tablet comprising or consisting essentially of:
about 30 w/w % to about 80 w/w % of an amorphous form of the free acid form of the compound of Formula Ib;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

In some embodiments, the method comprises administering to the patient a tablet comprising or consisting essentially of:
about 50 w/w % to about 70 w/w % of an amorphous form of the free acid form of the compound of Formula Ib;
about 25 w/w % to about 35 w/w % of microcrystalline cellulose;
about 5 w/w % to about 15 w/w % of crospovidone; and
about 1 w/w % to about 5 w/w % of magnesium stearate.

In some embodiments, the method comprises administering said tablet once every month or once every week.

In some embodiments, the method comprises administering to the patient a tablet comprising or consisting essentially of:
about 55 w/w % to about 65 w/w % of an amorphous form of the free acid form of the compound of Formula Ib;
about 25 w/w % to about 30 w/w % of microcrystalline cellulose;
about 8 w/w % to about 12 w/w % of crospovidone; and
about 1 w/w % to about 3 w/w % of magnesium stearate.

In some embodiments, the method comprises administering said tablet once every month or once every week.

In some embodiments, the method comprises administering to the patient a tablet comprising or consisting essentially of:
about 60 w/w % of an amorphous form of the free acid form of the compound of Formula Ib;
about 28.5 w/w % of microcrystalline cellulose;
about 10 w/w % of crospovidone; and
about 1.5 w/w % of magnesium stearate.

In some embodiments, the method comprises administering to the patient a tablet comprising or consisting essentially of:
about 60 w/w % of an amorphous form of the free acid form of the compound of Formula Ib;
about 28 w/w % of microcrystalline cellulose;
about 10 w/w % of crospovidone; and
about 2 w/w % of magnesium stearate.

In some embodiments, the method comprises administering said tablet once every month or once every week.

In some embodiments, about 1 mg to about 8000 mg of the compound of Formula Ia or Ib, or a pharmaceutically acceptable salt thereof, is administered to a patient in need thereof, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, about 6000 mg, about 6100 mg, about 6200 mg, about 6300 mg, about 6400 mg, about 6500 mg, about 6600 mg, about 6700 mg, about 6800 mg, about 6900 mg, about 7000 mg, about 7100 mg, about 7200 mg, about 7300 mg, about 7400 mg, about 7500 mg, about 7600 mg, about 7700 mg, about 7800 mg, about 7900 mg, or about 8000 mg of the compound of Formula Ia or Ib, or a pharmaceutically acceptable salt thereof, is administered to a patient in need thereof.

In some embodiments, about 225 mg to about 375 mg of the compound of Formula Ia or Ib, or a pharmaceutically acceptable salt thereof, is administered to a patient in need thereof. In some embodiments, about 250 mg to about 375 mg of the compound of Formula Ia or Ib, or a pharmaceutically acceptable salt thereof, is administered to a patient in need thereof. In some embodiments, about 275 mg to about 375 mg of the compound of Formula Ia or Ib, or a pharmaceutically acceptable salt thereof, is administered to a patient in need thereof. In some embodiments, about 300 mg to about 350 mg of the compound of Formula Ia or Ib, or a pharmaceutically acceptable salt thereof, is administered to a patient in need thereof.

In some embodiments, about 300 mg to about 310 mg of the compound of Formula Ia or Ib, or a pharmaceutically acceptable salt thereof, is administered to a patient in need thereof. In some embodiments, about 305 mg to about 307 mg of the compound of Formula Ia or Ib, or a pharmaceutically acceptable salt thereof, is administered to a patient in need thereof. In some embodiments, about 306 mg of the compound of Formula Ia or Ib, or a pharmaceutically acceptable salt thereof, is administered to a patient in need thereof. In some embodiments, about 306 mg of the free acid form of the compound of Formula Ia or Ib is administered to a patient in need thereof. In some embodiments, about 306 mg of the free acid form of the compound of Formula Ia is administered to a patient in need thereof. In some embodiments, about 306 mg of the free acid form of the compound of Formula Ib is administered to a patient in need thereof. In some embodiments, about 306 mg of an amorphous form of the free acid form of the compound of Formula Ia is administered to a patient in need thereof. In some embodiments, about 306 mg of an amorphous form of the free acid form of the compound of Formula Ib is administered to a patient in need thereof. In some embodiments, about 300 mg of the compound of Formula Ia or Ib, or a pharmaceutically acceptable salt thereof, is administered to a patient in need thereof. In some embodiments, about 300 mg of the free acid form of the compound of Formula Ia or Ib is administered to a patient in need thereof. In some embodiments, about 300 mg of the free acid form of the compound of Formula Ia is administered to a patient in need thereof. In some embodiments, about 300 mg of the free acid form of the compound of Formula Ib is administered to a patient in need thereof. In some embodiments, about 300 mg of an amorphous form of the free acid form of the compound of Formula Ia is administered to a patient in need thereof. In some embodiments, about 300 mg of an amorphous form of the free acid form of the compound of Formula Ib is administered to a patient in need thereof.

In some embodiments, about 100 mg to about 500 mg of the compound of Formula Ia or Ib, or a pharmaceutically acceptable salt thereof, is administered to a patient in need thereof. In some embodiments, about 200 mg to about 475 mg of the compound of Formula Ia or Ib, or a pharmaceutically acceptable salt thereof, is administered to a patient in need thereof. In some embodiments, about 200 mg to about 450 mg of the compound of Formula Ia or Ib, or a pharmaceutically acceptable salt thereof, is administered to a patient in need thereof. In some embodiments, about 250 mg to about 450 mg of the compound of Formula Ia or Ib, or a pharmaceutically acceptable salt thereof, is administered to a patient in need thereof. In some embodiments, about 300 mg to about 400 mg of the compound of Formula Ia or Ib, or a pharmaceutically acceptable salt thereof, is administered to a patient in need thereof. In some embodiments, about 325 mg to about 375 mg of the compound of Formula Ia or Ib, or a pharmaceutically acceptable salt thereof, is administered to a patient in need thereof. In some embodiments, about 340 mg to about 360 mg of the compound of Formula Ia or Ib, or a pharmaceutically acceptable salt thereof, is administered to a patient in need thereof. In some embodiments, about 345 mg to about 355 mg of the compound of Formula Ia or Ib, or a pharmaceutically acceptable salt thereof, is administered to a patient in need thereof. In some embodiments, about 349 mg to about 351 mg of the compound of Formula Ia or Ib, or a pharmaceutically acceptable salt thereof, is administered to a patient in need thereof. In some embodiments, about 350 mg of the compound of Formula Ia or Ib, or a pharmaceutically acceptable salt thereof, is administered to a patient in need thereof.

In some embodiments, about 100 mg to about 500 mg of the free acid form of the compound of Formula Ia or Ib is administered to a patient in need thereof. In some embodiments, about 200 mg to about 475 mg of the free acid form of the compound of Formula Ia or Ib is administered to a patient in need thereof. In some embodiments, about 200 mg to about 450 mg of the free acid form of the compound of Formula Ia or Ib is administered to a patient in need thereof. In some embodiments, about 250 mg to about 450 mg of the free acid form of the compound of Formula Ia or Ib is administered to a patient in need thereof. In some embodiments, about 300 mg to about 400 mg of the free acid form of the compound of Formula Ia or Ib is administered to a patient in need thereof. In some embodiments, about 325 mg to about 375 mg of the free acid form of the compound of Formula Ia or Ib is administered to a patient in need thereof. In some embodiments, about 340 mg to about 360 mg of the free acid form of the compound of Formula Ia or Ib is administered to a patient in need thereof. In some embodiments, about 345 mg to about 355 mg of the free acid form of the compound of Formula Ia or Ib is administered to a patient in need thereof. In some embodiments, about 349 mg to about 351 mg of the free acid form of the compound of Formula Ia or Ib is administered to a patient in need thereof. In some embodiments, about 350 mg of the compound of the free acid form of the compound of Formula Ia or Ib is administered to a patient in need thereof.

In some embodiments, about 100 mg to about 500 mg of the free acid form of the compound of Formula Ia is administered to a patient in need thereof. In some embodiments, about 200 mg to about 475 mg of the free acid form of the compound of Formula Ia is administered to a patient in need thereof. In some embodiments, about 200 mg to about 450 mg of the free acid form of the compound of Formula Ia is administered to a patient in need thereof. In some embodiments, about 250 mg to about 450 mg of the free acid form of the compound of Formula Ia is administered to a patient in need thereof. In some embodiments, about 300 mg to about 400 mg of the free acid form of the compound of Formula Ia is administered to a patient in need thereof. In some embodiments, about 325 mg to about 375 mg of the free acid form of the compound of Formula Ia is administered to a patient in need thereof. In some embodiments, about 340 mg to about 360 mg of the free acid form of the compound of Formula Ia is administered to a patient in need thereof. In some embodiments, about 345 mg to about 355 mg of the free acid form of the compound of Formula Ia is administered to a patient in need thereof. In some embodiments, about 349 mg to about 351 mg of the free acid form of the compound of Formula Ia is administered to a patient in need thereof. In some embodiments, about 350 mg of the compound of the free acid form of the compound of Formula Ia is administered to a patient in need thereof.

In some embodiments, about 100 mg to about 500 mg of the free acid form of the compound of Formula Ib is administered to a patient in need thereof. In some embodiments, about 200 mg to about 475 mg of the free acid form of the compound of Formula Ib is administered to a patient in need thereof. In some embodiments, about 200 mg to about 450 mg of the free acid form of the compound of Formula Ib is administered to a patient in need thereof. In some embodiments, about 250 mg to about 450 mg of the free acid form of the compound of Formula Ib is administered to a patient in need thereof. In some embodiments, about 300 mg to about 400 mg of the free acid form of the compound of Formula Ib is administered to a patient in need thereof. In some embodiments, about 325 mg to about 375 mg of the free acid form of the compound of Formula Ib is administered to a patient in need thereof. In some embodiments, about 340 mg to about 360 mg of the free acid form of the compound of Formula Ib is administered to a patient in need thereof. In some embodiments, about 345 mg to about 355 mg of the free acid form of the compound of Formula Ib is administered to a patient in need thereof. In some embodiments, about 349 mg to about 351 mg of the free acid form of the compound of Formula Ib is administered to a patient in need thereof. In some embodiments, about 350 mg of the compound of the free acid form of the compound of Formula Ib is administered to a patient in need thereof.

In some embodiments, about 100 mg to about 500 mg of an amorphous form of the free acid form of the compound of Formula Ia or Ib is administered to a patient in need thereof. In some embodiments, about 200 mg to about 475 mg of the free acid form of an amorphous form of the free acid form of the compound of Formula Ia or Ib is administered to a patient in need thereof. In some embodiments, about 200 mg to about 450 mg of an amorphous form of the free acid form of the compound of Formula Ia or Ib is administered to a patient in need thereof. In some embodiments, about 250 mg to about 450 mg of an amorphous form of the free acid form of the compound of Formula Ia or Ib is administered to a patient in need thereof. In some embodiments, about 300 mg to about 400 mg of an amorphous form of the free acid form of the compound of Formula Ia or Ib is administered to a patient in need thereof. In some embodiments, about 325 mg to about 375 mg of an amorphous form of the free acid form of the compound of Formula Ia or Ib is administered to a patient in need thereof. In some embodiments, about 340 mg to about 360 mg of an amorphous form of the free acid form of the compound of Formula Ia or Ib is administered to a patient in need thereof. In some embodiments, about 345 mg to about 355 mg of an amorphous form of the free acid form of the compound of Formula Ia or Ib is administered to a patient in need thereof. In some embodiments, about 349 mg to about 351 mg of an amorphous form of the free acid form of the compound of Formula Ia or Ib is administered to a patient in need thereof. In some embodiments, about 350 mg of the compound of an amorphous form of the free acid form of the compound of Formula Ia or Ib is administered to a patient in need thereof.

In some embodiments, about 100 mg to about 500 mg of an amorphous form of the free acid form of the compound of Formula Ia is administered to a patient in need thereof. In some embodiments, about 200 mg to about 475 mg of the free acid form of an amorphous form of the free acid form of the compound of Formula Ia is administered to a patient in need thereof. In some embodiments, about 200 mg to about 450 mg of an amorphous form of the free acid form of the compound of Formula Ia is administered to a patient in need thereof. In some embodiments, about 250 mg to about 450 mg of an amorphous form of the free acid form of the compound of Formula Ia is administered to a patient in need thereof. In some embodiments, about 300 mg to about 400 mg of an amorphous form of the free acid form of the compound of Formula Ia is administered to a patient in need thereof. In some embodiments, about 325 mg to about 375 mg of an amorphous form of the free acid form of the compound of Formula Ia is administered to a patient in need thereof. In some embodiments, about 340 mg to about 360 mg of an amorphous form of the free acid form of the compound of Formula Ia is administered to a patient in need thereof. In some embodiments, about 345 mg to about 355 mg of an amorphous form of the free acid form of the compound of Formula Ia is administered to a patient in need thereof. In some embodiments, about 349 mg to about 351 mg of an amorphous form of the free acid form of the compound of Formula Ia is administered to a patient in need thereof. In some embodiments, about 350 mg of the compound of an amorphous form of the free acid form of the compound of Formula Ia is administered to a patient in need thereof.

In some embodiments, about 100 mg to about 500 mg of an amorphous form of the free acid form of the compound of Formula Ib is administered to a patient in need thereof. In some embodiments, about 200 mg to about 475 mg of the free acid form of an amorphous form of the free acid form of the compound of Formula Ib is administered to a patient in need thereof. In some embodiments, about 200 mg to about 450 mg of an amorphous form of the free acid form of the compound of Formula Ib is administered to a patient in need thereof. In some embodiments, about 250 mg to about 450 mg of an amorphous form of the free acid form of the compound of Formula Ib is administered to a patient in need thereof. In some embodiments, about 300 mg to about 400 mg of an amorphous form of the free acid form of the compound of Formula Ib is administered to a patient in need thereof. In some embodiments, about 325 mg to about 375 mg of an amorphous form of the free acid form of the compound of Formula Ib is administered to a patient in need thereof. In some embodiments, about 340 mg to about 360 mg of an amorphous form of the free acid form of the compound of Formula Ib is administered to a patient in need thereof. In some embodiments, about 345 mg to about 355 mg of an amorphous form of the free acid form of the compound of Formula Ib is administered to a patient in need thereof. In some embodiments, about 349 mg to about 351 mg of an amorphous form of the free acid form of the compound of Formula Ib is administered to a patient in need thereof. In some embodiments, about 350 mg of the compound of an amorphous form of the free acid form of the compound of Formula Ib is administered to a patient in need thereof.

In some embodiments, about 500 mg to about 8000 mg of the compound of Formula Ia or Ib, or a pharmaceutically acceptable salt thereof, is administered to a patient in need thereof. In some embodiments, about 1000 mg to about 1500 mg of the compound of Formula Ia or Ib, or a pharmaceutically acceptable salt thereof, is administered to a patient in need thereof. In some embodiments, about 1300 mg to about 1500 mg of the compound of Formula Ia or Ib, or a pharmaceutically acceptable salt thereof, is administered to a patient in need thereof. In some embodiments, about 1350 mg to about 1450 mg of the compound of Formula Ia or Ib, or a pharmaceutically acceptable salt thereof, is administered to a patient in need thereof. In some embodiments, about 1375 mg to about 1425 mg of the compound of Formula Ia or Ib, or a pharmaceutically acceptable salt thereof, is administered to a patient in need thereof. In some embodiments, about 1390 mg to about 1400 mg of the compound of Formula Ia or Ib, or a pharmaceutically acceptable salt thereof, is administered to a patient in need thereof. In some embodiments, about 1395 mg to about 1405 mg of the compound of Formula Ia or Ib, or a pharmaceutically acceptable salt thereof, is administered to a patient in need thereof. In some embodiments, about 1399 mg to about 1401 mg of the compound of Formula Ia or Ib, or a pharmaceutically acceptable salt thereof, is administered to a patient in need thereof. In some embodiments, about 1400 mg of the compound of Formula Ia or Ib, or a pharmaceutically acceptable salt thereof, is administered to a patient in need thereof.

In some embodiments, about 500 mg to about 8000 mg of the free acid form of the compound of Formula Ia or Ib, is administered to a patient in need thereof. In some embodiments, about 1000 mg to about 1500 mg of the free acid form of the compound of Formula Ia or Ib, is administered to a patient in need thereof. In some embodiments, about 1300 mg to about 1500 mg of the free acid form of the compound of Formula Ia or Ib, is administered to a patient in need thereof. In some embodiments, about 1350 mg to about 1450 mg of the free acid form of the compound of Formula Ia or Ib, is administered to a patient in need thereof. In some embodiments, about 1375 mg to about 1425 mg of the free acid form of the compound of Formula Ia or Ib, is administered to a patient in need thereof. In some embodiments, about 1390 mg to about 1400 mg of the free acid form of the compound of Formula Ia or Ib, is administered to a patient in need thereof. In some embodiments, about 1395 mg to about 1405 mg of the free acid form of the compound of Formula Ia or Ib, is administered to a patient in need thereof. In some embodiments, about 1399 mg to about 1401 mg of the free acid form of the compound of Formula Ia or Ib, is administered to a patient in need thereof. In some embodiments, about 1400 mg of the free acid form of the compound of Formula Ia or Ib, is administered to a patient in need thereof.

In some embodiments, about 500 mg to about 8000 mg of the free acid form of the compound of Formula Ia, is administered to a patient in need thereof. In some embodiments, about 1000 mg to about 1500 mg of the free acid form of the compound of Formula Ia, is administered to a patient in need thereof. In some embodiments, about 1300 mg to about 1500 mg of the free acid form of the compound of Formula Ia, is administered to a patient in need thereof. In some embodiments, about 1350 mg to about 1450 mg of the free acid form of the compound of Formula Ia, is administered to a patient in need thereof. In some embodiments, about 1375 mg to about 1425 mg of the free acid form of the compound of Formula Ia, is administered to a patient in need thereof. In some embodiments, about 1390 mg to about 1400 mg of the free acid form of the compound of Formula Ia, is administered to a patient in need thereof. In some embodiments, about 1395 mg to about 1405 mg of the free acid form of the compound of Formula Ia, is administered to a patient in need thereof. In some embodiments, about 1399 mg to about 1401 mg of the free acid form of the compound of Formula Ia, is administered to a patient in need thereof. In some embodiments, about 1400 mg of the free acid form of the compound of Formula Ia, is administered to a patient in need thereof.

In some embodiments, about 500 mg to about 8000 mg of the free acid form of the compound of Formula Ib, is administered to a patient in need thereof. In some embodiments, about 1000 mg to about 1500 mg of the free acid form of the compound of Formula Ib, is administered to a patient in need thereof. In some embodiments, about 1300 mg to about 1500 mg of the free acid form of the compound of Formula Ib, is administered to a patient in need thereof. In some embodiments, about 1350 mg to about 1450 mg of the free acid form of the compound of Formula Ib, is administered to a patient in need thereof. In some embodiments, about 1375 mg to about 1425 mg of the free acid form of the compound of Formula Ib, is administered to a patient in need thereof. In some embodiments, about 1390 mg to about 1400 mg of the free acid form of the compound of Formula Ib, is administered to a patient in need thereof. In some embodiments, about 1395 mg to about 1405 mg of the free acid form of the compound of Formula Ib, is administered to a patient in need thereof. In some embodiments, about 1399 mg to about 1401 mg of the free acid form of the compound of Formula Ib, is administered to a patient in need thereof. In some embodiments, about 1400 mg of the free acid form of the compound of Formula Ib, is administered to a patient in need thereof.

In some embodiments, about 500 mg to about 8000 mg of an amorphous form of the free acid form of the compound of Formula Ia or Ib, is administered to a patient in need thereof. In some embodiments, about 1000 mg to about 1500 mg of an amorphous form of the free acid form of the compound of Formula Ia or Ib, is administered to a patient in need thereof. In some embodiments, about 1300 mg to about 1500 mg of an amorphous form of the free acid form of the compound of Formula Ia or Ib, is administered to a patient in need thereof. In some embodiments, about 1350 mg to about 1450 mg of an amorphous form of the free acid form of the compound of Formula Ia or Ib, is administered to a patient in need thereof. In some embodiments, about 1375 mg to about 1425 mg of an amorphous form of the free acid form of the compound of Formula Ia or Ib, is administered to a patient in need thereof. In some embodiments, about 1390 mg to about 1400 mg of an amorphous form of the free acid form of the compound of Formula Ia or Ib, is administered to a patient in need thereof. In some embodiments, about 1395 mg to about 1405 mg of an amorphous form of the free acid form of the compound of Formula Ia or Ib, is administered to a patient in need thereof. In some embodiments, about 1399 mg to about 1401 mg of an amorphous form of the free acid form of the compound of Formula Ia or Ib, is administered to a patient in need thereof. In some embodiments, about 1400 mg of an amorphous form of the free acid form of the compound of Formula Ia or Ib, is administered to a patient in need thereof.

In some embodiments, about 500 mg to about 8000 mg of an amorphous form of the free acid form of the compound of Formula Ia, is administered to a patient in need thereof. In some embodiments, about 1000 mg to about 1500 mg of an amorphous form of the free acid form of the compound of Formula Ia, is administered to a patient in need thereof. In some embodiments, about 1300 mg to about 1500 mg of an amorphous form of the free acid form of the compound of Formula Ia, is administered to a patient in need thereof. In some embodiments, about 1350 mg to about 1450 mg of an amorphous form of the free acid form of the compound of Formula Ia, is administered to a patient in need thereof. In some embodiments, about 1375 mg to about 1425 mg of an amorphous form of the free acid form of the compound of Formula Ia, is administered to a patient in need thereof. In some embodiments, about 1390 mg to about 1400 mg of an amorphous form of the free acid form of the compound of Formula Ia, is administered to a patient in need thereof. In some embodiments, about 1395 mg to about 1405 mg of an amorphous form of the free acid form of the compound of Formula Ia, is administered to a patient in need thereof. In some embodiments, about 1399 mg to about 1401 mg of an amorphous form of the free acid form of the compound of Formula Ia, is administered to a patient in need thereof. In some embodiments, about 1400 mg of an amorphous form of the free acid form of the compound of Formula Ia, is administered to a patient in need thereof.

In some embodiments, about 500 mg to about 8000 mg of an amorphous form of the free acid form of the compound of Formula Ib, is administered to a patient in need thereof. In some embodiments, about 1000 mg to about 1500 mg of an amorphous form of the free acid form of the compound of Formula Ib, is administered to a patient in need thereof. In some embodiments, about 1300 mg to about 1500 mg of an amorphous form of the free acid form of the compound of Formula Ib, is administered to a patient in need thereof. In some embodiments, about 1350 mg to about 1450 mg of an amorphous form of the free acid form of the compound of Formula Ib, is administered to a patient in need thereof. In some embodiments, about 1375 mg to about 1425 mg of an amorphous form of the free acid form of the compound of Formula Ib, is administered to a patient in need thereof. In some embodiments, about 1390 mg to about 1400 mg of an amorphous form of the free acid form of the compound of Formula Ib, is administered to a patient in need thereof. In some embodiments, about 1395 mg to about 1405 mg of an amorphous form of the free acid form of the compound of Formula Ib, is administered to a patient in need thereof. In some embodiments, about 1399 mg to about 1401 mg of an amorphous form of the free acid form of the compound of Formula Ib, is administered to a patient in need thereof. In some embodiments, about 1400 mg of an amorphous form of the free acid form of the compound of Formula Ib, is administered to a patient in need thereof.

In some embodiments, about 2500 mg to about 3000 mg of the compound of Formula Ia or Ib, or a pharmaceutically acceptable salt thereof, is administered to a patient in need thereof. In some embodiments, about 2700 mg to about 2900 mg of the compound of Formula Ia or Ib, or a pharmaceutically acceptable salt thereof, is administered to a patient in need thereof. In some embodiments, about 2750 mg to about 2950 mg of the compound of Formula Ia or Ib, or a pharmaceutically acceptable salt thereof, is administered to a patient in need thereof. In some embodiments, about 2775 mg to about 2925 mg of the compound of Formula Ia or Ib, or a pharmaceutically acceptable salt thereof, is administered to a patient in need thereof. In some embodiments, about 2790 mg to about 2810 mg of the compound of Formula Ia or Ib, or a pharmaceutically acceptable salt thereof, is administered to a patient in need thereof. In some embodiments, about 2795 mg to about 2805 mg of the compound of Formula Ia or Ib, or a pharmaceutically acceptable salt thereof, is administered to a patient in need thereof. In some embodiments, about 2799 mg to about 2801 mg of the compound of Formula Ia or Ib, or a pharmaceutically acceptable salt thereof, is administered to a patient in need thereof. In some embodiments, about 2800 mg of the compound of Formula Ia or Ib, or a pharmaceutically acceptable salt thereof, is administered to a patient in need thereof.

In some embodiments, about 2500 mg to about 3000 of the free acid form of the compound of Formula Ia or Ib, is administered to a patient in need thereof. In some embodiments, about 2700 mg to about 2900 mg of the free acid form of the compound of Formula Ia or Ib, is administered to a patient in need thereof. In some embodiments, about 2750 mg to about 2950 mg of the free acid form of the compound of Formula Ia or Ib, is administered to a patient in need thereof. In some embodiments, about 2775 mg to about 2925 mg of the free acid form of the compound of Formula Ia or Ib, is administered to a patient in need thereof. In some embodiments, about 2790 mg to about 2810 mg of the free acid form of the compound of Formula Ia or Ib, is administered to a patient in need thereof. In some embodiments, about 2795 mg to about 2805 mg of the free acid form of the compound of Formula Ia or Ib, is administered to a patient in need thereof. In some embodiments, about 2799 mg to about 2801 mg of the free acid form of the compound of Formula Ia or Ib, is administered to a patient in need thereof. In some embodiments, about 2800 mg of the free acid form of the compound of Formula Ia or Ib, is administered to a patient in need thereof.

In some embodiments, about 2500 mg to about 3000 of the free acid form of the compound of Formula Ia, is administered to a patient in need thereof. In some embodiments, about 2700 mg to about 2900 mg of the free acid form of the compound of Formula Ia, is administered to a patient in need thereof. In some embodiments, about 2750 mg to about 2950 mg of the free acid form of the compound of Formula Ia, is administered to a patient in need thereof. In some embodiments, about 2775 mg to about 2925 mg of the free acid form of the compound of Formula Ia, is administered to a patient in need thereof. In some embodiments, about 2790 mg to about 2810 mg of the free acid form of the compound of Formula Ia, is administered to a patient in need thereof. In some embodiments, about 2795 mg to about 2805 mg of the free acid form of the compound of Formula Ia, is administered to a patient in need thereof. In some embodiments, about 2799 mg to about 2801 mg of the free acid form of the compound of Formula Ia, is administered to a patient in need thereof. In some embodiments, about 2800 mg of the free acid form of the compound of Formula Ia, is administered to a patient in need thereof.

In some embodiments, about 2500 mg to about 3000 of the free acid form of the compound of Formula Ib, is administered to a patient in need thereof. In some embodiments, about 2700 mg to about 2900 mg of the free acid form of the compound of Formula Ib, is administered to a patient in need thereof. In some embodiments, about 2750 mg to about 2950 mg of the free acid form of the compound of Formula Ib, is administered to a patient in need thereof. In some embodiments, about 2775 mg to about 2925 mg of the free acid form of the compound of Formula Ib, is administered to a patient in need thereof. In some embodiments, about 2790 mg to about 2810 mg of the free acid form of the compound of Formula Ib, is administered to a patient in need thereof. In some embodiments, about 2795 mg to about 2805 mg of the free acid form of the compound of Formula Ib, is administered to a patient in need thereof. In some embodiments, about 2799 mg to about 2801 mg of the free acid form of the compound of Formula Ib, is administered to a patient in need thereof. In some embodiments, about 2800 mg of the free acid form of the compound of Formula Ib, is administered to a patient in need thereof.

In some embodiments, about 2500 mg to about 3000 of an amorphous form of the free acid form of the compound of Formula Ia or Ib, is administered to a patient in need thereof. In some embodiments, about 2700 mg to about 2900 mg of an amorphous form of the free acid form of the compound of Formula Ia or Ib, is administered to a patient in need thereof. In some embodiments, about 2750 mg to about 2950 mg of an amorphous form of the free acid form of the compound of Formula Ia or Ib, is administered to a patient in need thereof. In some embodiments, about 2775 mg to about 2925 mg of an amorphous form of the free acid form of the compound of Formula Ia or Ib, is administered to a patient in need thereof. In some embodiments, about 2790 mg to about 2810 mg of an amorphous form of the free acid form of the compound of Formula Ia or Ib, is administered to a patient in need thereof. In some embodiments, about 2795 mg to about 2805 mg of an amorphous form of the free acid form of the compound of Formula Ia or Ib, is administered to a patient in need thereof. In some embodiments, about 2799 mg to about 2801 mg of an amorphous form of the free acid form of the compound of Formula Ia or Ib, is administered to a patient in need thereof. In some embodiments, about 2800 mg of an amorphous form of the free acid form of the compound of Formula Ia or Ib, is administered to a patient in need thereof.

In some embodiments, about 2500 mg to about 3000 of an amorphous form of the free acid form of the compound of Formula Ia, is administered to a patient in need thereof. In some embodiments, about 2700 mg to about 2900 mg of an amorphous form of the free acid form of the compound of Formula Ia, is administered to a patient in need thereof. In some embodiments, about 2750 mg to about 2950 mg of an amorphous form of the free acid form of the compound of Formula Ia, is administered to a patient in need thereof. In some embodiments, about 2775 mg to about 2925 mg of an amorphous form of the free acid form of the compound of Formula Ia, is administered to a patient in need thereof. In some embodiments, about 2790 mg to about 2810 mg of an amorphous form of the free acid form of the compound of Formula Ia, is administered to a patient in need thereof. In some embodiments, about 2795 mg to about 2805 mg of an amorphous form of the free acid form of the compound of Formula Ia, is administered to a patient in need thereof. In some embodiments, about 2799 mg to about 2801 mg of an amorphous form of the free acid form of the compound of Formula Ia, is administered to a patient in need thereof. In some embodiments, about 2800 mg of an amorphous form of the free acid form of the compound of Formula Ia, is administered to a patient in need thereof.

In some embodiments, about 2500 mg to about 3000 of an amorphous form of the free acid form of the compound of Formula Ib, is administered to a patient in need thereof. In some embodiments, about 2700 mg to about 2900 mg of an amorphous form of the free acid form of the compound of Formula Ib, is administered to a patient in need thereof. In some embodiments, about 2750 mg to about 2950 mg of an amorphous form of the free acid form of the compound of Formula Ib, is administered to a patient in need thereof. In some embodiments, about 2775 mg to about 2925 mg of an amorphous form of the free acid form of the compound of Formula Ib, is administered to a patient in need thereof. In some embodiments, about 2790 mg to about 2810 mg of an amorphous form of the free acid form of the compound of Formula Ib, is administered to a patient in need thereof. In some embodiments, about 2795 mg to about 2805 mg of an amorphous form of the free acid form of the compound of Formula Ib, is administered to a patient in need thereof. In some embodiments, about 2799 mg to about 2801 mg of an amorphous form of the free acid form of the compound of Formula Ib, is administered to a patient in need thereof. In some embodiments, about 2800 mg of an amorphous form of the free acid form of the compound of Formula Ib, is administered to a patient in need thereof.

In some embodiments, about 6000 mg to about 8000 mg of the compound of Formula Ia or Ib, or a pharmaceutically acceptable salt thereof, is administered to a patient in need thereof. In some embodiments, about 6500 mg to about 8000 mg of the compound of Formula Ia or Ib, or a pharmaceutically acceptable salt thereof, is administered to a patient in need thereof. In some embodiments, about 6500 mg to about 7500 mg of the compound of Formula Ia or Ib, or a pharmaceutically acceptable salt thereof, is administered to a patient in need thereof. In some embodiments, about 6900 mg to about 7100 mg of the compound of Formula Ia or Ib, or a pharmaceutically acceptable salt thereof, is administered to a patient in need thereof. In some embodiments, about 6925 mg to about 7025 mg of the compound of Formula Ia or Ib, or a pharmaceutically acceptable salt thereof, is administered to a patient in need thereof. In some embodiments, about 6990 mg to about 7010 mg of the compound of Formula Ia or Ib, or a pharmaceutically acceptable salt thereof, is administered to a patient in need thereof. In some embodiments, about 6995 mg to about 7005 mg of the compound of Formula Ia or Ib, or a pharmaceutically acceptable salt thereof, is administered to a patient in need thereof. In some embodiments, about 6999 mg to about 7001 mg of the compound of Formula Ia or Ib, or a pharmaceutically acceptable salt thereof, is administered to a patient in need thereof. In some embodiments, about 7000 mg of the compound of Formula Ia or Ib, or a pharmaceutically acceptable salt thereof, is administered to a patient in need thereof.

In some embodiments, about 6000 mg to about 8000 mg of the free acid form of the compound of Formula Ia or Ib, is administered to a patient in need thereof. In some embodiments, about 6500 mg to about 8000 mg of the free acid form of the compound of Formula Ia or Ib, is administered to a patient in need thereof. In some embodiments, about 6500 mg to about 7500 mg of the free acid form of the compound of Formula Ia or Ib, is administered to a patient in need thereof. In some embodiments, about 6900 mg to about 7100 mg of the free acid form of the compound of Formula Ia or Ib, is administered to a patient in need thereof. In some embodiments, about 6925 mg to about 7025 mg of the free acid form of the compound of Formula Ia or Ib, is administered to a patient in need thereof. In some embodiments, about 6990 mg to about 7010 mg of the free acid form of the compound of Formula Ia or Ib, is administered to a patient in need thereof. In some embodiments, about 6995 mg to about 7005 mg of the free acid form of the compound of Formula Ia or Ib, is administered to a patient in need thereof. In some embodiments, about 6999 mg to about 7001 mg of the free acid form of the compound of Formula Ia or Ib, is administered to a patient in need thereof. In some embodiments, about 7000 mg of the free acid form of the compound of Formula Ia or Ib, is administered to a patient in need thereof.

In some embodiments, about 6000 mg to about 8000 mg of the free acid form of the compound of Formula Ia, is administered to a patient in need thereof. In some embodiments, about 6500 mg to about 8000 mg of the free acid form of the compound of Formula Ia, is administered to a patient in need thereof. In some embodiments, about 6500 mg to about 7500 mg of the free acid form of the compound of Formula Ia, is administered to a patient in need thereof. In some embodiments, about 6900 mg to about 7100 mg of the free acid form of the compound of Formula Ia, is administered to a patient in need thereof. In some embodiments, about 6925 mg to about 7025 mg of the free acid form of the compound of Formula Ia, is administered to a patient in need thereof. In some embodiments, about 6990 mg to about 7010 mg of the free acid form of the compound of Formula Ia, is administered to a patient in need thereof. In some embodiments, about 6995 mg to about 7005 mg of the free acid form of the compound of Formula Ia, is administered to a patient in need thereof. In some embodiments, about 6999 mg to about 7001 mg of the free acid form of the compound of Formula Ia, is administered to a patient in need thereof. In some embodiments, about 7000 mg of the free acid form of the compound of Formula Ia, is administered to a patient in need thereof.

In some embodiments, about 6000 mg to about 8000 mg of the free acid form of the compound of Formula Ib, is administered to a patient in need thereof. In some embodiments, about 6500 mg to about 8000 mg of the free acid form of the compound of Formula Ib, is administered to a patient in need thereof. In some embodiments, about 6500 mg to about 7500 mg of the free acid form of the compound of Formula Ib, is administered to a patient in need thereof. In some embodiments, about 6900 mg to about 7100 mg of the free acid form of the compound of Formula Ib, is administered to a patient in need thereof. In some embodiments, about 6925 mg to about 7025 mg of the free acid form of the compound of Formula Ib, is administered to a patient in need thereof. In some embodiments, about 6990 mg to about 7010 mg of the free acid form of the compound of Formula Ib, is administered to a patient in need thereof. In some embodiments, about 6995 mg to about 7005 mg of the free acid form of the compound of Formula Ib, is administered to a patient in need thereof. In some embodiments, about 6999 mg to about 7001 mg of the free acid form of the compound of Formula Ib, is administered to a patient in need thereof. In some embodiments, about 7000 mg of the free acid form of the compound of Formula Ib, is administered to a patient in need thereof.

In some embodiments, about 6000 mg to about 8000 mg of an amorphous form of the free acid form of the compound of Formula Ia or Ib, is administered to a patient in need thereof. In some embodiments, about 6500 mg to about 8000 mg of an amorphous form of the free acid form of the compound of Formula Ia or Ib, is administered to a patient in need thereof. In some embodiments, about 6500 mg to about 7500 mg of an amorphous form of the free acid form of the compound of Formula Ia or Ib, is administered to a patient in need thereof. In some embodiments, about 6900 mg to about 7100 mg of an amorphous form of the free acid form of the compound of Formula Ia or Ib, is administered to a patient in need thereof. In some embodiments, about 6925 mg to about 7025 mg of an amorphous form of the free acid form of the compound of Formula Ia or Ib, is administered to a patient in need thereof. In some embodiments, about 6990 mg to about 7010 mg of an amorphous form of the free acid form of the compound of Formula Ia or Ib, is administered to a patient in need thereof. In some embodiments, about 6995 mg to about 7005 mg of an amorphous form of the free acid form of the compound of Formula Ia or Ib, is administered to a patient in need thereof. In some embodiments, about 6999 mg to about 7001 mg of an amorphous form of the free acid form of the compound of Formula Ia or Ib, is administered to a patient in need thereof. In some embodiments, about 7000 mg of an amorphous form of the free acid form of the compound of Formula Ia or Ib, is administered to a patient in need thereof.

In some embodiments, about 6000 mg to about 8000 mg of an amorphous form of the free acid form of the compound of Formula Ia, is administered to a patient in need thereof. In some embodiments, about 6500 mg to about 8000 mg of an amorphous form of the free acid form of the compound of Formula Ia, is administered to a patient in need thereof. In some embodiments, about 6500 mg to about 7500 mg of an amorphous form of the free acid form of the compound of Formula Ia, is administered to a patient in need thereof. In some embodiments, about 6900 mg to about 7100 mg of an amorphous form of the free acid form of the compound of Formula Ia, is administered to a patient in need thereof. In some embodiments, about 6925 mg to about 7025 mg of an amorphous form of the free acid form of the compound of Formula Ia, is administered to a patient in need thereof. In some embodiments, about 6990 mg to about 7010 mg of an amorphous form of the free acid form of the compound of Formula Ia, is administered to a patient in need thereof. In some embodiments, about 6995 mg to about 7005 mg of an amorphous form of the free acid form of the compound of Formula Ia, is administered to a patient in need thereof. In some embodiments, about 6999 mg to about 7001 mg of an amorphous form of the free acid form of the compound of Formula Ia, is administered to a patient in need thereof. In some embodiments, about 7000 mg of an amorphous form of the free acid form of the compound of Formula Ia, is administered to a patient in need thereof.

In some embodiments, about 6000 mg to about 8000 mg of an amorphous form of the free acid form of the compound of Formula Ib, is administered to a patient in need thereof. In some embodiments, about 6500 mg to about 8000 mg of an amorphous form of the free acid form of the compound of Formula Ib, is administered to a patient in need thereof. In some embodiments, about 6500 mg to about 7500 mg of an amorphous form of the free acid form of the compound of Formula Ib, is administered to a patient in need thereof. In some embodiments, about 6900 mg to about 7100 mg of an amorphous form of the free acid form of the compound of Formula Ib, is administered to a patient in need thereof. In some embodiments, about 6925 mg to about 7025 mg of an amorphous form of the free acid form of the compound of Formula Ib, is administered to a patient in need thereof. In some embodiments, about 6990 mg to about 7010 mg of an amorphous form of the free acid form of the compound of Formula Ib, is administered to a patient in need thereof. In some embodiments, about 6995 mg to about 7005 mg of an amorphous form of the free acid form of the compound of Formula Ib, is administered to a patient in need thereof. In some embodiments, about 6999 mg to about 7001 mg of an amorphous form of the free acid form of the compound of Formula Ib, is administered to a patient in need thereof. In some embodiments, about 7000 mg of an amorphous form of the free acid form of the compound of Formula Ib, is administered to a patient in need thereof.

In some embodiments, about 1 mg to about 8000 mg of the compound of Formula Ia or Ib, or a pharmaceutically acceptable salt thereof, is administered to a patient in need thereof once or twice every 25 to 40 days, for example, once or twice every 25, 26, 27, 28, 20, 30, 31, 32, 33, 34, 35, 36, 37, 38, 39, or 40 days.

In some embodiments, the methods provided herein comprise administering to the patient one or more of the tablets provided herein (e.g., administration of one tablet, two tablets, three tablets, and the like), every 25 to 40 days, for example, once or twice every 25, 26, 27, 28, 20, 30, 31, 32, 33, 34, 35, 36, 37, 38, 39, or 40 days.

In some embodiments, the methods provided herein comprise administering to the patient one or more of the tablets provided herein (e.g., administration of one tablet, two tablets, three tablets, and the like), once or twice every 25 to 40 days, for example, once or twice every 25, 26, 27, 28, 20, 30, 31, 32, 33, 34, 35, 36, 37, 38, 39, or 40 days.

In some embodiments, about 225 mg to about 375 mg of the compound of Formula Ia or Ib, or a pharmaceutically acceptable salt thereof, is administered to a patient in need thereof, once or twice every 25 to 40 days. In some embodiments, about 250 mg to about 375 mg of the compound of Formula Ia or Ib, or a pharmaceutically acceptable salt thereof, is administered to a patient in need thereof, once or twice every 25 to 40 days. In some embodiments, about 275 mg to about 375 mg of the compound of Formula Ia or Ib, or a pharmaceutically acceptable salt thereof, is administered to a patient in need thereof, once or twice every 25 to 40 days. In some embodiments, about 300 mg to about 350 mg of the compound of Formula Ia or Ib, or a pharmaceutically acceptable salt thereof, is administered to a patient in need thereof, once or twice every 25 to 40 days.

In some embodiments, about 300 mg to about 310 mg of the compound of Formula Ia or Ib, or a pharmaceutically acceptable salt thereof, is administered to a patient in need thereof, once or twice every 25 to 40 days. In some embodiments, about 305 mg to about 307 mg of the compound of Formula Ia or Ib, or a pharmaceutically acceptable salt thereof, is administered to a patient in need thereof, once or twice every 25 to 40 days. In some embodiments, about 306 mg of the compound of Formula Ia or Ib, or a pharmaceutically acceptable salt thereof, is administered to a patient in need thereof, once or twice every 25 to 40 days. In some embodiments, about 306 mg of the free acid form of the compound of Formula Ia or Ib is administered to a patient in need thereof, once or twice every 25 to 40 days. In some embodiments, about 306 mg of the free acid form of the compound of Formula Ia is administered to a patient in need thereof, once or twice every 25 to 40 days. In some embodiments, about 306 mg of the free acid form of the compound of Formula Ib is administered to a patient in need thereof, once or twice every 25 to 40 days. In some embodiments, about 306 mg of an amorphous form of the free acid form of the compound of Formula Ia is administered to a patient in need thereof, once or twice every 25 to 40 days. In some embodiments, about 306 mg of an amorphous form of the free acid form of the compound of Formula Ib is administered to a patient in need thereof, once or twice every 25 to 40 days. In some embodiments, about 300 mg of the compound of Formula Ia or Ib, or a pharmaceutically acceptable salt thereof, is administered to a patient in need thereof, once or twice every 25 to 40 days. In some embodiments, about 300 mg of the free acid form of the compound of Formula Ia or Ib is administered to a patient in need thereof, once or twice every 25 to 40 days. In some embodiments, about 300 mg of the free acid form of the compound of Formula Ia is administered to a patient in need thereof, once or twice every 25 to 40 days. In some embodiments, about 300 mg of the free acid form of the compound of Formula Ib is administered to a patient in need thereof, once or twice every 25 to 40 days. In some embodiments, about 300 mg of an amorphous form of the free acid form of the compound of Formula Ia is administered to a patient in need thereof, once or twice every 25 to 40 days. In some embodiments, about 300 mg of an amorphous form of the free acid form of the compound of Formula Ib is administered to a patient in need thereof, once or twice every 25 to 40 days.

In some embodiments, about 100 mg to about 500 mg of the compound of Formula Ia or Ib, or a pharmaceutically acceptable salt thereof, is administered to a patient in need thereof, once or twice every 25 to 40 days. In some embodiments, about 200 mg to about 475 mg of the compound of Formula Ia or Ib, or a pharmaceutically acceptable salt thereof, is administered to a patient in need thereof, once or twice every 25 to 40 days. In some embodiments, about 200 mg to about 450 mg of the compound of Formula Ia or Ib, or a pharmaceutically acceptable salt thereof, is administered to a patient in need thereof, once or twice every 25 to 40 days. In some embodiments, about 250 mg to about 450 mg of the compound of Formula Ia or Ib, or a pharmaceutically acceptable salt thereof, is administered to a patient in need thereof, once or twice every 25 to 40 days. In some embodiments, about 300 mg to about 400 mg of the compound of Formula Ia or Ib, or a pharmaceutically acceptable salt thereof, is administered to a patient in need thereof, once or twice every 25 to 40 days. In some embodiments, about 325 mg to about 375 mg of the compound of Formula Ia or Ib, or a pharmaceutically acceptable salt thereof, is administered to a patient in need thereof, once or twice every 25 to 40 days. In some embodiments, about 340 mg to about 360 mg of the compound of Formula Ia or Ib, or a pharmaceutically acceptable salt thereof, is administered to a patient in need thereof, once or twice every 25 to 40 days. In some embodiments, about 345 mg to about 355 mg of the compound of Formula Ia or Ib, or a pharmaceutically acceptable salt thereof, is administered to a patient in need thereof, once or twice every 25 to 40 days. In some embodiments, about 349 mg to about 351 mg of the compound of Formula Ia or Ib, or a pharmaceutically acceptable salt thereof, is administered to a patient in need thereof, once or twice every 25 to 40 days. In some embodiments, about 350 mg of the compound of Formula Ia or Ib, or a pharmaceutically acceptable salt thereof, is administered to a patient in need thereof, once or twice every 25 to 40 days.

In some embodiments, about 100 mg to about 500 mg of the free acid form of the compound of Formula Ia or Ib is administered to a patient in need thereof, once or twice every 25 to 40 days. In some embodiments, about 200 mg to about 475 mg of the free acid form of the compound of Formula Ia or Ib is administered to a patient in need thereof, once or twice every 25 to 40 days. In some embodiments, about 200 mg to about 450 mg of the free acid form of the compound of Formula Ia or Ib is administered to a patient in need thereof, once or twice every 25 to 40 days. In some embodiments, about 250 mg to about 450 mg of the free acid form of the compound of Formula Ia or Ib is administered to a patient in need thereof, once or twice every 25 to 40 days. In some embodiments, about 300 mg to about 400 mg of the free acid form of the compound of Formula Ia or Ib is administered to a patient in need thereof, once or twice every 25 to 40 days. In some embodiments, about 325 mg to about 375 mg of the free acid form of the compound of Formula Ia or Ib is administered to a patient in need thereof, once or twice every 25 to 40 days. In some embodiments, about 340 mg to about 360 mg of the free acid form of the compound of Formula Ia or Ib is administered to a patient in need thereof, once or twice every 25 to 40 days. In some embodiments, about 345 mg to about 355 mg of the free acid form of the compound of Formula Ia or Ib is administered to a patient in need thereof, once or twice every 25 to 40 days. In some embodiments, about 349 mg to about 351 mg of the free acid form of the compound of Formula Ia or Ib is administered to a patient in need thereof, once or twice every 25 to 40 days. In some embodiments, about 350 mg of the compound of the free acid form of the compound of Formula Ia or Ib is administered to a patient in need thereof, once or twice every 25 to 40 days.

In some embodiments, about 100 mg to about 500 mg of the free acid form of the compound of Formula Ia is administered to a patient in need thereof, once or twice every 25 to 40 days. In some embodiments, about 200 mg to about 475 mg of the free acid form of the compound of Formula Ia is administered to a patient in need thereof, once or twice every 25 to 40 days. In some embodiments, about 200 mg to about 450 mg of the free acid form of the compound of Formula Ia is administered to a patient in need thereof, once or twice every 25 to 40 days. In some embodiments, about 250 mg to about 450 mg of the free acid form of the compound of Formula Ia is administered to a patient in need thereof, once or twice every 25 to 40 days. In some embodiments, about 300 mg to about 400 mg of the free acid form of the compound of Formula Ia is administered to a patient in need thereof, once or twice every 25 to 40 days. In some embodiments, about 325 mg to about 375 mg of the free acid form of the compound of Formula Ia is administered to a patient in need thereof, once or twice every 25 to 40 days. In some embodiments, about 340 mg to about 360 mg of the free acid form of the compound of Formula Ia is administered to a patient in need thereof, once or twice every 25 to 40 days. In some embodiments, about 345 mg to about 355 mg of the free acid form of the compound of Formula Ia is administered to a patient in need thereof, once or twice every 25 to 40 days. In some embodiments, about 349 mg to about 351 mg of the free acid form of the compound of Formula Ia is administered to a patient in need thereof, once or twice every 25 to 40 days. In some embodiments, about 350 mg of the compound of the free acid form of the compound of Formula Ia is administered to a patient in need thereof, once or twice every 25 to 40 days.

In some embodiments, about 100 mg to about 500 mg of the free acid form of the compound of Formula Ib is administered to a patient in need thereof, once or twice every 25 to 40 days. In some embodiments, about 200 mg to about 475 mg of the free acid form of the compound of Formula Ib is administered to a patient in need thereof, once or twice every 25 to 40 days. In some embodiments, about 200 mg to about 450 mg of the free acid form of the compound of Formula Ib is administered to a patient in need thereof, once or twice every 25 to 40 days. In some embodiments, about 250 mg to about 450 mg of the free acid form of the compound of Formula Ib is administered to a patient in need thereof, once or twice every 25 to 40 days. In some embodiments, about 300 mg to about 400 mg of the free acid form of the compound of Formula Ib is administered to a patient in need thereof, once or twice every 25 to 40 days. In some embodiments, about 325 mg to about 375 mg of the free acid form of the compound of Formula Ib is administered to a patient in need thereof, once or twice every 25 to 40 days. In some embodiments, about 340 mg to about 360 mg of the free acid form of the compound of Formula Ib is administered to a patient in need thereof, once or twice every 25 to 40 days. In some embodiments, about 345 mg to about 355 mg of the free acid form of the compound of Formula Ib is administered to a patient in need thereof, once or twice every 25 to 40 days. In some embodiments, about 349 mg to about 351 mg of the free acid form of the compound of Formula Ib is administered to a patient in need thereof, once or twice every 25 to 40 days. In some embodiments, about 350 mg of the compound of the free acid form of the compound of Formula Ib is administered to a patient in need thereof, once or twice every 25 to 40 days.

In some embodiments, about 100 mg to about 500 mg of an amorphous form of the free acid form of the compound of Formula Ia or Ib is administered to a patient in need thereof, once or twice every 25 to 40 days. In some embodiments, about 200 mg to about 475 mg of the free acid form of an amorphous form of the free acid form of the compound of Formula Ia or Ib is administered to a patient in need thereof, once or twice every 25 to 40 days. In some embodiments, about 200 mg to about 450 mg of an amorphous form of the free acid form of the compound of Formula Ia or Ib is administered to a patient in need thereof, once or twice every 25 to 40 days. In some embodiments, about 250 mg to about 450 mg of an amorphous form of the free acid form of the compound of Formula Ia or Ib is administered to a patient in need thereof, once or twice every 25 to 40 days. In some embodiments, about 300 mg to about 400 mg of an amorphous form of the free acid form of the compound of Formula Ia or Ib is administered to a patient in need thereof, once or twice every 25 to 40 days. In some embodiments, about 325 mg to about 375 mg of an amorphous form of the free acid form of the compound of Formula Ia or Ib is administered to a patient in need thereof, once or twice every 25 to 40 days. In some embodiments, about 340 mg to about 360 mg of an amorphous form of the free acid form of the compound of Formula Ia or Ib is administered to a patient in need thereof, once or twice every 25 to 40 days. In some embodiments, about 345 mg to about 355 mg of an amorphous form of the free acid form of the compound of Formula Ia or Ib is administered to a patient in need thereof, once or twice every 25 to 40 days. In some embodiments, about 349 mg to about 351 mg of an amorphous form of the free acid form of the compound of Formula Ia or Ib is administered to a patient in need thereof, once or twice every 25 to 40 days. In some embodiments, about 350 mg of the compound of an amorphous form of the free acid form of the compound of Formula Ia or Ib is administered to a patient in need thereof, once or twice every 25 to 40 days.

In some embodiments, about 100 mg to about 500 mg of an amorphous form of the free acid form of the compound of Formula Ia is administered to a patient in need thereof, once or twice every 25 to 40 days. In some embodiments, about 200 mg to about 475 mg of the free acid form of an amorphous form of the free acid form of the compound of Formula Ia is administered to a patient in need thereof, once or twice every 25 to 40 days. In some embodiments, about 200 mg to about 450 mg of an amorphous form of the free acid form of the compound of Formula Ia is administered to a patient in need thereof, once or twice every 25 to 40 days. In some embodiments, about 250 mg to about 450 mg of an amorphous form of the free acid form of the compound of Formula Ia is administered to a patient in need thereof, once or twice every 25 to 40 days. In some embodiments, about 300 mg to about 400 mg of an amorphous form of the free acid form of the compound of Formula Ia is administered to a patient in need thereof, once or twice every 25 to 40 days. In some embodiments, about 325 mg to about 375 mg of an amorphous form of the free acid form of the compound of Formula Ia is administered to a patient in need thereof, once or twice every 25 to 40 days. In some embodiments, about 340 mg to about 360 mg of an amorphous form of the free acid form of the compound of Formula Ia is administered to a patient in need thereof, once or twice every 25 to 40 days. In some embodiments, about 345 mg to about 355 mg of an amorphous form of the free acid form of the compound of Formula Ia is administered to a patient in need thereof, once or twice every 25 to 40 days. In some embodiments, about 349 mg to about 351 mg of an amorphous form of the free acid form of the compound of Formula Ia is administered to a patient in need thereof, once or twice every 25 to 40 days. In some embodiments, about 350 mg of the compound of an amorphous form of the free acid form of the compound of Formula Ia is administered to a patient in need thereof, once or twice every 25 to 40 days.

In some embodiments, about 100 mg to about 500 mg of an amorphous form of the free acid form of the compound of Formula Ib is administered to a patient in need thereof, once or twice every 25 to 40 days. In some embodiments, about 200 mg to about 475 mg of the free acid form of an amorphous form of the free acid form of the compound of Formula Ib is administered to a patient in need thereof, once or twice every 25 to 40 days. In some embodiments, about 200 mg to about 450 mg of an amorphous form of the free acid form of the compound of Formula Ib is administered to a patient in need thereof, once or twice every 25 to 40 days. In some embodiments, about 250 mg to about 450 mg of an amorphous form of the free acid form of the compound of Formula Ib is administered to a patient in need thereof, once or twice every 25 to 40 days. In some embodiments, about 300 mg to about 400 mg of an amorphous form of the free acid form of the compound of Formula Ib is administered to a patient in need thereof, once or twice every 25 to 40 days. In some embodiments, about 325 mg to about 375 mg of an amorphous form of the free acid form of the compound of Formula Ib is administered to a patient in need thereof, once or twice every 25 to 40 days. In some embodiments, about 340 mg to about 360 mg of an amorphous form of the free acid form of the compound of Formula Ib is administered to a patient in need thereof, once or twice every 25 to 40 days. In some embodiments, about 345 mg to about 355 mg of an amorphous form of the free acid form of the compound of Formula Ib is administered to a patient in need thereof, once or twice every 25 to 40 days. In some embodiments, about 349 mg to about 351 mg of an amorphous form of the free acid form of the compound of Formula Ib is administered to a patient in need thereof, once or twice every 25 to 40 days. In some embodiments, about 350 mg of the compound of an amorphous form of the free acid form of the compound of Formula Ib is administered to a patient in need thereof, once or twice every 25 to 40 days.

In some embodiments, about 500 mg to about 1000 mg of the compound of Formula Ia or Ib, or a pharmaceutically acceptable salt thereof, is administered to a patient in need thereof, once or twice every 25 to 40 days. In some embodiments, about 600 mg to about 800 mg of the compound of Formula Ia or Ib, or a pharmaceutically acceptable salt thereof, is administered to a patient in need thereof, once or twice every 25 to 40 days. In some embodiments, about 600 mg to about 750 mg of the compound of Formula Ia or Ib, or a pharmaceutically acceptable salt thereof, is administered to a patient in need thereof, once or twice every 25 to 40 days. In some embodiments, about 650 mg to about 750 mg of the compound of Formula Ia or Ib, or a pharmaceutically acceptable salt thereof, is administered to a patient in need thereof, once or twice every 25 to 40 days. In some embodiments, about 675 mg to about 725 mg of the compound of Formula Ia or Ib, or a pharmaceutically acceptable salt thereof, is administered to a patient in need thereof, once or twice every 25 to 40 days. In some embodiments, about 690 mg to about 710 mg of the compound of Formula Ia or Ib, or a pharmaceutically acceptable salt thereof, is administered to a patient in need thereof, once or twice every 25 to 40 days. In some embodiments, about 695 mg to about 705 mg of the compound of Formula Ia or Ib, or a pharmaceutically acceptable salt thereof, is administered to a patient in need thereof, once or twice every 25 to 40 days. In some embodiments, about 699 mg to about 701 mg of the compound of Formula Ia or Ib, or a pharmaceutically acceptable salt thereof, is administered to a patient in need thereof, once or twice every 25 to 40 days. In some embodiments, about 700 mg of the compound of Formula Ia or Ib, or a pharmaceutically acceptable salt thereof, is administered to a patient in need thereof, once or twice every 25 to 40 days.

In some embodiments, about 500 mg to about 1000 mg of the free acid form of the compound of Formula Ia or Ib is administered to a patient in need thereof, once or twice every 25 to 40 days. In some embodiments, about 600 mg to about 800 mg of the free acid form of the compound of Formula Ia or Ib is administered to a patient in need thereof, once or twice every 25 to 40 days. In some embodiments, about 600 mg to about 750 mg of the free acid form of the compound of Formula Ia or Ib is administered to a patient in need thereof, once or twice every 25 to 40 days. In some embodiments, about 650 mg to about 750 mg of the free acid form of the compound of Formula Ia or Ib is administered to a patient in need thereof, once or twice every 25 to 40 days. In some embodiments, about 675 mg to about 725 mg of the free acid form of the compound of Formula Ia or Ib is administered to a patient in need thereof, once or twice every 25 to 40 days. In some embodiments, about 690 mg to about 710 mg of the free acid form of the compound of Formula Ia or Ib is administered to a patient in need thereof, once or twice every 25 to 40 days. In some embodiments, about 695 mg to about 705 mg of the free acid form of the compound of Formula Ia or Ib is administered to a patient in need thereof, once or twice every 25 to 40 days. In some embodiments, about 699 mg to about 701 mg of the free acid form of the compound of Formula Ia or Ib is administered to a patient in need thereof, once or twice every 25 to 40 days. In some embodiments, about 700 mg of the free acid form of the compound of Formula Ia or Ib is administered to a patient in need thereof, once or twice every 25 to 40 days.

In some embodiments, about 500 mg to about 1000 mg of the free acid form of the compound of Formula Ia is administered to a patient in need thereof, once or twice every 25 to 40 days. In some embodiments, about 600 mg to about 800 mg of the free acid form of the compound of Formula Ia is administered to a patient in need thereof, once or twice every 25 to 40 days. In some embodiments, about 600 mg to about 750 mg of the free acid form of the compound of Formula Ia is administered to a patient in need thereof, once or twice every 25 to 40 days. In some embodiments, about 650 mg to about 750 mg of the free acid form of the compound of Formula Ia is administered to a patient in need thereof, once or twice every 25 to 40 days. In some embodiments, about 675 mg to about 725 mg of the free acid form of the compound of Formula Ia is administered to a patient in need thereof, once or twice every 25 to 40 days. In some embodiments, about 690 mg to about 710 mg of the free acid form of the compound of Formula Ia is administered to a patient in need thereof, once or twice every 25 to 40 days. In some embodiments, about 695 mg to about 705 mg of the free acid form of the compound of Formula Ia is administered to a patient in need thereof, once or twice every 25 to 40 days. In some embodiments, about 699 mg to about 701 mg of the free acid form of the compound of Formula Ia is administered to a patient in need thereof, once or twice every 25 to 40 days. In some embodiments, about 700 mg of the free acid form of the compound of Formula Ia is administered to a patient in need thereof, once or twice every 25 to 40 days.

In some embodiments, about 500 mg to about 1000 mg of the free acid form of the compound of Formula Ib is administered to a patient in need thereof, once or twice every 25 to 40 days. In some embodiments, about 600 mg to about 800 mg of the free acid form of the compound of Formula Ib is administered to a patient in need thereof, once or twice every 25 to 40 days. In some embodiments, about 600 mg to about 750 mg of the free acid form of the compound of Formula Ib is administered to a patient in need thereof, once or twice every 25 to 40 days. In some embodiments, about 650 mg to about 750 mg of the free acid form of the compound of Formula Ib is administered to a patient in need thereof, once or twice every 25 to 40 days. In some embodiments, about 675 mg to about 725 mg of the free acid form of the compound of Formula Ib is administered to a patient in need thereof, once or twice every 25 to 40 days. In some embodiments, about 690 mg to about 710 mg of the free acid form of the compound of Formula Ib is administered to a patient in need thereof, once or twice every 25 to 40 days. In some embodiments, about 695 mg to about 705 mg of the free acid form of the compound of Formula Ib is administered to a patient in need thereof, once or twice every 25 to 40 days. In some embodiments, about 699 mg to about 701 mg of the free acid form of the compound of Formula Ib is administered to a patient in need thereof, once or twice every 25 to 40 days. In some embodiments, about 700 mg of the free acid form of the compound of Formula Ib is administered to a patient in need thereof, once or twice every 25 to 40 days.

In some embodiments, about 500 mg to about 1000 mg of an amorphous form of the free acid form of the compound of Formula Ia or Ib is administered to a patient in need thereof, once or twice every 25 to 40 days. In some embodiments, about 600 mg to about 800 mg of an amorphous form of the free acid form of the compound of Formula Ia or Ib is administered to a patient in need thereof, once or twice every 25 to 40 days. In some embodiments, about 600 mg to about 750 mg of an amorphous form of the free acid form of the compound of Formula Ia or Ib is administered to a patient in need thereof, once or twice every 25 to 40 days. In some embodiments, about 650 mg to about 750 mg of the free acid form of an amorphous form of the free acid form of the compound of Formula Ia or Ib is administered to a patient in need thereof, once or twice every 25 to 40 days. In some embodiments, about 675 mg to about 725 mg of an amorphous form of the free acid form of the compound of Formula Ia or Ib is administered to a patient in need thereof, once or twice every 25 to 40 days. In some embodiments, about 690 mg to about 710 mg of an amorphous form of the free acid form of the compound of Formula Ia or Ib is administered to a patient in need thereof, once or twice every 25 to 40 days. In some embodiments, about 695 mg to about 705 mg of an amorphous form of the free acid form of the compound of Formula Ia or Ib is administered to a patient in need thereof, once or twice every 25 to 40 days. In some embodiments, about 699 mg to about 701 mg of an amorphous form of the free acid form of the compound of Formula Ia or Ib is administered to a patient in need thereof, once or twice every 25 to 40 days. In some embodiments, about 700 mg of an amorphous form of the free acid form of the compound of Formula Ia or Ib is administered to a patient in need thereof, once or twice every 25 to 40 days.

In some embodiments, about 500 mg to about 1000 mg of an amorphous form of the free acid form of the compound of Formula Ia is administered to a patient in need thereof, once or twice every 25 to 40 days. In some embodiments, about 600 mg to about 800 mg of an amorphous form of the free acid form of the compound of Formula Ia is administered to a patient in need thereof, once or twice every 25 to 40 days. In some embodiments, about 600 mg to about 750 mg of an amorphous form of the free acid form of the compound of Formula Ia is administered to a patient in need thereof, once or twice every 25 to 40 days. In some embodiments, about 650 mg to about 750 mg of the free acid form of an amorphous form of the free acid form of the compound of Formula Ia is administered to a patient in need thereof, once or twice every 25 to 40 days. In some embodiments, about 675 mg to about 725 mg of an amorphous form of the free acid form of the compound of Formula Ia is administered to a patient in need thereof, once or twice every 25 to 40 days. In some embodiments, about 690 mg to about 710 mg of an amorphous form of the free acid form of the compound of Formula Ia is administered to a patient in need thereof, once or twice every 25 to 40 days. In some embodiments, about 695 mg to about 705 mg of an amorphous form of the free acid form of the compound of Formula Ia is administered to a patient in need thereof, once or twice every 25 to 40 days. In some embodiments, about 699 mg to about 701 mg of an amorphous form of the free acid form of the compound of Formula Ia is administered to a patient in need thereof, once or twice every 25 to 40 days. In some embodiments, about 700 mg of an amorphous form of the free acid form of the compound of Formula Ia is administered to a patient in need thereof, once or twice every 25 to 40 days.

In some embodiments, about 500 mg to about 1000 mg of an amorphous form of the free acid form of the compound of Formula Ib is administered to a patient in need thereof, once or twice every 25 to 40 days. In some embodiments, about 600 mg to about 800 mg of an amorphous form of the free acid form of the compound of Formula Ib is administered to a patient in need thereof, once or twice every 25 to 40 days. In some embodiments, about 600 mg to about 750 mg of an amorphous form of the free acid form of the compound of Formula Ib is administered to a patient in need thereof, once or twice every 25 to 40 days. In some embodiments, about 650 mg to about 750 mg of the free acid form of an amorphous form of the free acid form of the compound of Formula Ib is administered to a patient in need thereof, once or twice every 25 to 40 days. In some embodiments, about 675 mg to about 725 mg of an amorphous form of the free acid form of the compound of Formula Ib is administered to a patient in need thereof, once or twice every 25 to 40 days. In some embodiments, about 690 mg to about 710 mg of an amorphous form of the free acid form of the compound of Formula Ib is administered to a patient in need thereof, once or twice every 25 to 40 days. In some embodiments, about 695 mg to about 705 mg of an amorphous form of the free acid form of the compound of Formula Ib is administered to a patient in need thereof, once or twice every 25 to 40 days. In some embodiments, about 699 mg to about 701 mg of an amorphous form of the free acid form of the compound of Formula Ib is administered to a patient in need thereof, once or twice every 25 to 40 days. In some embodiments, about 700 mg of an amorphous form of the free acid form of the compound of Formula Ib is administered to a patient in need thereof, once or twice every 25 to 40 days.

In some embodiments, the compound of Formula Ia or Ib, or a pharmaceutically acceptable salt thereof, is administered once every 25 to 40 days. In some embodiments, the free acid form of the compound of Formula Ia or Ib, is administered once every 25 to 40 days. In some embodiments, the free acid form of the compound of Formula Ia, is administered once every 25 to 40 days. In some embodiments, the free acid form of the compound of Formula Ib, is administered once every 25 to 40 days. In some embodiments, an amorphous form of the free acid form of the compound of Formula Ia or Ib, is administered once every 25 to 40 days. In some embodiments, an amorphous form of the free acid form of the compound of Formula Ia, is administered once every 25 to 40 days. In some embodiments, an amorphous form of the free acid form of the compound of Formula Ib, is administered once every 25 to 40 days.

In some embodiments, the compound of Formula Ia or Ib, or a pharmaceutically acceptable salt thereof, is administered twice every 25 to 40 days. In some embodiments, the free acid form of the compound of Formula Ia or Ib, is administered twice every 25 to 40 days. In some embodiments, the free acid form of the compound of Formula Ia, is administered twice every 25 to 40 days. In some embodiments, the free acid form of the compound of Formula Ib, is administered twice every 25 to 40 days. In some embodiments, an amorphous form of the free acid form of the compound of Formula Ia or Ib, is administered twice every 25 to 40 days. In some embodiments, an amorphous form of the free acid form of the compound of Formula Ia, is administered twice every 25 to 40 days. In some embodiments, an amorphous form of the free acid form of the compound of Formula Ib, is administered twice every 25 to 40 days.

In some embodiments, about 225 mg to about 375 mg of the compound of Formula Ia or Ib, or a pharmaceutically acceptable salt thereof, is administered to a patient in need thereof, once or twice every month. In some embodiments, about 250 mg to about 375 mg of the compound of Formula Ia or Ib, or a pharmaceutically acceptable salt thereof, is administered to a patient in need thereof, once or twice every month. In some embodiments, about 275 mg to about 375 mg of the compound of Formula Ia or Ib, or a pharmaceutically acceptable salt thereof, is administered to a patient in need thereof, once or twice every month. In some embodiments, about 300 mg to about 350 mg of the compound of Formula Ia or Ib, or a pharmaceutically acceptable salt thereof, is administered to a patient in need thereof, once or twice every month.

In some embodiments, about 300 mg to about 310 mg of the compound of Formula Ia or Ib, or a pharmaceutically acceptable salt thereof, is administered to a patient in need thereof, once or twice every month. In some embodiments, about 305 mg to about 307 mg of the compound of Formula Ia or Ib, or a pharmaceutically acceptable salt thereof, is administered to a patient in need thereof, once or twice every month. In some embodiments, about 306 mg of the compound of Formula Ia or Ib, or a pharmaceutically acceptable salt thereof, is administered to a patient in need thereof, once or twice every month. In some embodiments, about 306 mg of the free acid form of the compound of Formula Ia or Ib is administered to a patient in need thereof, once or twice every month. In some embodiments, about 306 mg of the free acid form of the compound of Formula Ia is administered to a patient in need thereof, once or twice every month. In some embodiments, about 306 mg of the free acid form of the compound of Formula Ib is administered to a patient in need thereof, once or twice every month. In some embodiments, about 306 mg of an amorphous form of the free acid form of the compound of Formula Ia is administered to a patient in need thereof, once or twice every month. In some embodiments, about 306 mg of an amorphous form of the free acid form of the compound of Formula Ib is administered to a patient in need thereof, once or twice every month.

In some embodiments, about 300 mg of the compound of Formula Ia or Ib, or a pharmaceutically acceptable salt thereof, is administered to a patient in need thereof, once or twice every month. In some embodiments, about 300 mg of the free acid form of the compound of Formula Ia or Ib is administered to a patient in need thereof, once or twice every month. In some embodiments, about 300 mg of the free acid form of the compound of Formula Ia is administered to a patient in need thereof, once or twice every month. In some embodiments, about 300 mg of the free acid form of the compound of Formula Ib is administered to a patient in need thereof, once or twice every month. In some embodiments, about 300 mg of an amorphous form of the free acid form of the compound of Formula Ia is administered to a patient in need thereof, once or twice every month. In some embodiments, about 300 mg of an amorphous form of the free acid form of the compound of Formula Ib is administered to a patient in need thereof, once or twice every month.

In some embodiments, about 600 mg to about 800 mg of the compound of Formula Ia or Ib, or a pharmaceutically acceptable salt thereof, is administered to a patient in need thereof, once or twice every month. In some embodiments, about 600 mg to about 800 mg of the free acid form of the compound of Formula Ia or Ib is administered to a patient in need thereof, once or twice every month. In some embodiments, about 600 mg to about 800 mg of the free acid form of the compound of Formula Ia is administered to a patient in need thereof, once or twice every month. In some embodiments, about 600 mg to about 800 mg of the free acid form of the compound of Formula Ib is administered to a patient in need thereof, once or twice every month. In some embodiments, about 600 mg to about 800 mg of an amorphous form of the free acid form of the compound of Formula Ia is administered to a patient in need thereof, once or twice every month. In some embodiments, about 600 mg to about 800 mg of an amorphous form of the free acid form of the compound of Formula Ib is administered to a patient in need thereof, once or twice every month.

In some embodiments, about 675 mg to about 725 mg of the compound of Formula Ia or Ib, or a pharmaceutically acceptable salt thereof, is administered to a patient in need thereof, once or twice every month. In some embodiments, about 675 mg to about 725 mg of the free acid form of the compound of Formula Ia or Ib is administered to a patient in need thereof, once or twice every month. In some embodiments, about 675 mg to about 725 mg of the free acid form of the compound of Formula Ia is administered to a patient in need thereof, once or twice every month. In some embodiments, about 675 mg to about 725 mg of the free acid form of the compound of Formula Ib is administered to a patient in need thereof, once or twice every month. In some embodiments, about 675 mg to about 725 mg of an amorphous form of the free acid form of the compound of Formula Ia is administered to a patient in need thereof, once or twice every month. In some embodiments, about 675 mg to about 725 mg of an amorphous form of the free acid form of the compound of Formula Ib is administered to a patient in need thereof, once or twice every month.

In some embodiments, about 690 mg to about 710 mg of the compound of Formula Ia or Ib, or a pharmaceutically acceptable salt thereof, is administered to a patient in need thereof, once or twice every month. In some embodiments, about 690 mg to about 710 mg of the free acid form of the compound of Formula Ia or Ib is administered to a patient in need thereof, once or twice every month. In some embodiments, about 690 mg to about 710 mg of the free acid form of the compound of Formula Ia is administered to a patient in need thereof, once or twice every month. In some embodiments, about 690 mg to about 710 mg of the free acid form of the compound of Formula Ib is administered to a patient in need thereof, once or twice every month. In some embodiments, about 690 mg to about 710 mg of an amorphous form of the free acid form of the compound of Formula Ia is administered to a patient in need thereof, once or twice every month. In some embodiments, about 690 mg to about 710 mg of an amorphous form of the free acid form of the compound of Formula Ib is administered to a patient in need thereof, once or twice every month.

In some embodiments, about 700 mg of the compound of Formula Ia or Ib, or a pharmaceutically acceptable salt thereof, is administered to a patient in need thereof, once or twice every month. In some embodiments, about 700 mg of the free acid form of the compound of Formula Ia or Ib is administered to a patient in need thereof, once or twice every month. In some embodiments, about 700 mg of the free acid form of the compound of Formula Ia is administered to a patient in need thereof, once or twice every month. In some embodiments, about 700 mg of the free acid form of the compound of Formula Ib is administered to a patient in need thereof, once or twice every month. In some embodiments, about 700 mg of an amorphous form of the free acid form of the compound of Formula Ia is administered to a patient in need thereof, once or twice every month. In some embodiments, about 700 mg of an amorphous form of the free acid form of the compound of Formula Ib is administered to a patient in need thereof, once or twice every month.

In some embodiments, about 100 mg to about 500 mg of the compound of Formula Ia or Ib, or a pharmaceutically acceptable salt thereof, is administered to a patient in need thereof, once or twice every month. In some embodiments, about 200 mg to about 475 mg of the compound of Formula Ia or Ib, or a pharmaceutically acceptable salt thereof, is administered to a patient in need thereof, once or twice every month. In some embodiments, about 200 mg to about 450 mg of the compound of Formula Ia or Ib, or a pharmaceutically acceptable salt thereof, is administered to a patient in need thereof, once or twice every month. In some embodiments, about 250 mg to about 450 mg of the compound of Formula Ia or Ib, or a pharmaceutically acceptable salt thereof, is administered to a patient in need thereof, once or twice every month. In some embodiments, about 300 mg to about 400 mg of the compound of Formula Ia or Ib, or a pharmaceutically acceptable salt thereof, is administered to a patient in need thereof, once or twice every month. In some embodiments, about 325 mg to about 375 mg of the compound of Formula Ia or Ib, or a pharmaceutically acceptable salt thereof, is administered to a patient in need thereof, once or twice every month. In some embodiments, about 340 mg to about 360 mg of the compound of Formula Ia or Ib, or a pharmaceutically acceptable salt thereof, is administered to a patient in need thereof, once or twice every month. In some embodiments, about 345 mg to about 355 mg of the compound of Formula Ia or Ib, or a pharmaceutically acceptable salt thereof, is administered to a patient in need thereof, once or twice every month. In some embodiments, about 349 mg to about 351 mg of the compound of Formula Ia or Ib, or a pharmaceutically acceptable salt thereof, is administered to a patient in need thereof, once or twice every month. In some embodiments, about 350 mg of the compound of Formula Ia or Ib, or a pharmaceutically acceptable salt thereof, is administered to a patient in need thereof, once or twice every month.

In some embodiments, about 100 mg to about 500 mg of the free acid form of the compound of Formula Ia or Ib is administered to a patient in need thereof, once or twice every month. In some embodiments, about 200 mg to about 475 mg of the free acid form of the compound of Formula Ia or Ib is administered to a patient in need thereof, once or twice every month. In some embodiments, about 200 mg to about 450 mg of the free acid form of the compound of Formula Ia or Ib is administered to a patient in need thereof, once or twice every month. In some embodiments, about 250 mg to about 450 mg of the free acid form of the compound of Formula Ia or Ib is administered to a patient in need thereof, once or twice every month. In some embodiments, about 300 mg to about 400 mg of the free acid form of the compound of Formula Ia or Ib is administered to a patient in need thereof, once or twice every month. In some embodiments, about 325 mg to about 375 mg of the free acid form of the compound of Formula Ia or Ib is administered to a patient in need thereof, once or twice every month. In some embodiments, about 340 mg to about 360 mg of the free acid form of the compound of Formula Ia or Ib is administered to a patient in need thereof, once or twice every month. In some embodiments, about 345 mg to about 355 mg of the free acid form of the compound of Formula Ia or Ib is administered to a patient in need thereof, once or twice every month. In some embodiments, about 349 mg to about 351 mg of the free acid form of the compound of Formula Ia or Ib is administered to a patient in need thereof, once or twice every month. In some embodiments, about 350 mg of the compound of the free acid form of the compound of Formula Ia or Ib is administered to a patient in need thereof, once or twice every month.

In some embodiments, about 100 mg to about 500 mg of the free acid form of the compound of Formula Ia is administered to a patient in need thereof, once or twice every month. In some embodiments, about 200 mg to about 475 mg of the free acid form of the compound of Formula Ia is administered to a patient in need thereof, once or twice every month. In some embodiments, about 200 mg to about 450 mg of the free acid form of the compound of Formula Ia is administered to a patient in need thereof, once or twice every month. In some embodiments, about 250 mg to about 450 mg of the free acid form of the compound of Formula Ia is administered to a patient in need thereof, once or twice every month. In some embodiments, about 300 mg to about 400 mg of the free acid form of the compound of Formula Ia is administered to a patient in need thereof, once or twice every month. In some embodiments, about 325 mg to about 375 mg of the free acid form of the compound of Formula Ia is administered to a patient in need thereof, once or twice every month. In some embodiments, about 340 mg to about 360 mg of the free acid form of the compound of Formula Ia is administered to a patient in need thereof, once or twice every month. In some embodiments, about 345 mg to about 355 mg of the free acid form of the compound of Formula Ia is administered to a patient in need thereof, once or twice every month. In some embodiments, about 349 mg to about 351 mg of the free acid form of the compound of Formula Ia is administered to a patient in need thereof, once or twice every month. In some embodiments, about 350 mg of the compound of the free acid form of the compound of Formula Ia is administered to a patient in need thereof, once or twice every month.

In some embodiments, about 100 mg to about 500 mg of the free acid form of the compound of Formula Ib is administered to a patient in need thereof, once or twice every month. In some embodiments, about 200 mg to about 475 mg of the free acid form of the compound of Formula Ib is administered to a patient in need thereof, once or twice every month. In some embodiments, about 200 mg to about 450 mg of the free acid form of the compound of Formula Ib is administered to a patient in need thereof, once or twice every month. In some embodiments, about 250 mg to about 450 mg of the free acid form of the compound of Formula Ib is administered to a patient in need thereof, once or twice every month. In some embodiments, about 300 mg to about 400 mg of the free acid form of the compound of Formula Ib is administered to a patient in need thereof, once or twice every month. In some embodiments, about 325 mg to about 375 mg of the free acid form of the compound of Formula Ib is administered to a patient in need thereof, once or twice every month. In some embodiments, about 340 mg to about 360 mg of the free acid form of the compound of Formula Ib is administered to a patient in need thereof, once or twice every month. In some embodiments, about 345 mg to about 355 mg of the free acid form of the compound of Formula Ib is administered to a patient in need thereof, once or twice every month. In some embodiments, about 349 mg to about 351 mg of the free acid form of the compound of Formula Ib is administered to a patient in need thereof, once or twice every month. In some embodiments, about 350 mg of the compound of the free acid form of the compound of Formula Ib is administered to a patient in need thereof, once or twice every month.

In some embodiments, about 100 mg to about 500 mg of an amorphous form of the free acid form of the compound of Formula Ia or Ib is administered to a patient in need thereof, once or twice every month. In some embodiments, about 200 mg to about 475 mg of the free acid form of an amorphous form of the free acid form of the compound of Formula Ia or Ib is administered to a patient in need thereof, once or twice every month. In some embodiments, about 200 mg to about 450 mg of an amorphous form of the free acid form of the compound of Formula Ia or Ib is administered to a patient in need thereof, once or twice every month. In some embodiments, about 250 mg to about 450 mg of an amorphous form of the free acid form of the compound of Formula Ia or Ib is administered to a patient in need thereof, once or twice every month. In some embodiments, about 300 mg to about 400 mg of an amorphous form of the free acid form of the compound of Formula Ia or Ib is administered to a patient in need thereof, once or twice every month. In some embodiments, about 325 mg to about 375 mg of an amorphous form of the free acid form of the compound of Formula Ia or Ib is administered to a patient in need thereof, once or twice every month. In some embodiments, about 340 mg to about 360 mg of an amorphous form of the free acid form of the compound of Formula Ia or Ib is administered to a patient in need thereof, once or twice every month. In some embodiments, about 345 mg to about 355 mg of an amorphous form of the free acid form of the compound of Formula Ia or Ib is administered to a patient in need thereof, once or twice every month. In some embodiments, about 349 mg to about 351 mg of an amorphous form of the free acid form of the compound of Formula Ia or Ib is administered to a patient in need thereof, once or twice every month. In some embodiments, about 350 mg of the compound of an amorphous form of the free acid form of the compound of Formula Ia or Ib is administered to a patient in need thereof, once or twice every month.

In some embodiments, about 100 mg to about 500 mg of an amorphous form of the free acid form of the compound of Formula Ia is administered to a patient in need thereof, once or twice every month. In some embodiments, about 200 mg to about 475 mg of the free acid form of an amorphous form of the free acid form of the compound of Formula Ia is administered to a patient in need thereof, once or twice every month. In some embodiments, about 200 mg to about 450 mg of an amorphous form of the free acid form of the compound of Formula Ia is administered to a patient in need thereof, once or twice every month. In some embodiments, about 250 mg to about 450 mg of an amorphous form of the free acid form of the compound of Formula Ia is administered to a patient in need thereof, once or twice every month. In some embodiments, about 300 mg to about 400 mg of an amorphous form of the free acid form of the compound of Formula Ia is administered to a patient in need thereof, once or twice every month. In some embodiments, about 325 mg to about 375 mg of an amorphous form of the free acid form of the compound of Formula Ia is administered to a patient in need thereof, once or twice every month. In some embodiments, about 340 mg to about 360 mg of an amorphous form of the free acid form of the compound of Formula Ia is administered to a patient in need thereof, once or twice every month. In some embodiments, about 345 mg to about 355 mg of an amorphous form of the free acid form of the compound of Formula Ia is administered to a patient in need thereof, once or twice every month. In some embodiments, about 349 mg to about 351 mg of an amorphous form of the free acid form of the compound of Formula Ia is administered to a patient in need thereof, once or twice every month. In some embodiments, about 350 mg of the compound of an amorphous form of the free acid form of the compound of Formula Ia is administered to a patient in need thereof, once or twice every month.

In some embodiments, about 100 mg to about 500 mg of an amorphous form of the free acid form of the compound of Formula Ib is administered to a patient in need thereof, once or twice every month. In some embodiments, about 200 mg to about 475 mg of the free acid form of an amorphous form of the free acid form of the compound of Formula Ib is administered to a patient in need thereof, once or twice every month. In some embodiments, about 200 mg to about 450 mg of an amorphous form of the free acid form of the compound of Formula Ib is administered to a patient in need thereof, once or twice every month. In some embodiments, about 250 mg to about 450 mg of an amorphous form of the free acid form of the compound of Formula Ib is administered to a patient in need thereof, once or twice every month. In some embodiments, about 300 mg to about 400 mg of an amorphous form of the free acid form of the compound of Formula Ib is administered to a patient in need thereof, once or twice every month. In some embodiments, about 325 mg to about 375 mg of an amorphous form of the free acid form of the compound of Formula Ib is administered to a patient in need thereof, once or twice every month. In some embodiments, about 340 mg to about 360 mg of an amorphous form of the free acid form of the compound of Formula Ib is administered to a patient in need thereof, once or twice every month. In some embodiments, about 345 mg to about 355 mg of an amorphous form of the free acid form of the compound of Formula Ib is administered to a patient in need thereof, once or twice every month. In some embodiments, about 349 mg to about 351 mg of an amorphous form of the free acid form of the compound of Formula Ib is administered to a patient in need thereof, once or twice every month. In some embodiments, about 350 mg of the compound of an amorphous form of the free acid form of the compound of Formula Ib is administered to a patient in need thereof, once or twice every month.

In some embodiments, about 500 mg to about 8000 mg of the compound of Formula Ia or Ib, or a pharmaceutically acceptable salt thereof, is administered to a patient in need thereof, once or twice every month. In some embodiments, about 1000 mg to about 1500 mg of the compound of Formula Ia or Ib, or a pharmaceutically acceptable salt thereof, is administered to a patient in need thereof, once or twice every month. In some embodiments, about 1300 mg to about 1500 mg of the compound of Formula Ia or Ib, or a pharmaceutically acceptable salt thereof, is administered to a patient in need thereof, once or twice every month. In some embodiments, about 1350 mg to about 1450 mg of the compound of Formula Ia or Ib, or a pharmaceutically acceptable salt thereof, is administered to a patient in need thereof, once or twice every month. In some embodiments, about 1375 mg to about 1425 mg of the compound of Formula Ia or Ib, or a pharmaceutically acceptable salt thereof, is administered to a patient in need thereof, once or twice every month. In some embodiments, about 1390 mg to about 1400 mg of the compound of Formula Ia or Ib, or a pharmaceutically acceptable salt thereof, is administered to a patient in need thereof, once or twice every month. In some embodiments, about 1395 mg to about 1405 mg of the compound of Formula Ia or Ib, or a pharmaceutically acceptable salt thereof, is administered to a patient in need thereof, once or twice every month. In some embodiments, about 1399 mg to about 1401 mg of the compound of Formula Ia or Ib, or a pharmaceutically acceptable salt thereof, is administered to a patient in need thereof, once or twice every month. In some embodiments, about 1400 mg of the compound of Formula Ia or Ib, or a pharmaceutically acceptable salt thereof, is administered to a patient in need thereof, once or twice every month.

In some embodiments, about 500 mg to about 8000 mg of the free acid form of the compound of Formula Ia or Ib, is administered to a patient in need thereof, once or twice every month. In some embodiments, about 1000 mg to about 1500 mg of the free acid form of the compound of Formula Ia or Ib, is administered to a patient in need thereof, once or twice every month. In some embodiments, about 1300 mg to about 1500 mg of the free acid form of the compound of Formula Ia or Ib, is administered to a patient in need thereof, once or twice every month. In some embodiments, about 1350 mg to about 1450 mg of the free acid form of the compound of Formula Ia or Ib, is administered to a patient in need thereof, once or twice every month. In some embodiments, about 1375 mg to about 1425 mg of the free acid form of the compound of Formula Ia or Ib, is administered to a patient in need thereof, once or twice every month. In some embodiments, about 1390 mg to about 1400 mg of the free acid form of the compound of Formula Ia or Ib, is administered to a patient in need thereof, once or twice every month. In some embodiments, about 1395 mg to about 1405 mg of the free acid form of the compound of Formula Ia or Ib, is administered to a patient in need thereof, once or twice every month. In some embodiments, about 1399 mg to about 1401 mg of the free acid form of the compound of Formula Ia or Ib, is administered to a patient in need thereof, once or twice every month. In some embodiments, about 1400 mg of the free acid form of the compound of Formula Ia or Ib, is administered to a patient in need thereof, once or twice every month.

In some embodiments, about 500 mg to about 8000 mg of the free acid form of the compound of Formula Ia, is administered to a patient in need thereof, once or twice every month. In some embodiments, about 1000 mg to about 1500 mg of the free acid form of the compound of Formula Ia, is administered to a patient in need thereof, once or twice every month. In some embodiments, about 1300 mg to about 1500 mg of the free acid form of the compound of Formula Ia, is administered to a patient in need thereof, once or twice every month. In some embodiments, about 1350 mg to about 1450 mg of the free acid form of the compound of Formula Ia, is administered to a patient in need thereof, once or twice every month. In some embodiments, about 1375 mg to about 1425 mg of the free acid form of the compound of Formula Ia, is administered to a patient in need thereof, once or twice every month. In some embodiments, about 1390 mg to about 1400 mg of the free acid form of the compound of Formula Ia, is administered to a patient in need thereof, once or twice every month. In some embodiments, about 1395 mg to about 1405 mg of the free acid form of the compound of Formula Ia, is administered to a patient in need thereof, once or twice every month. In some embodiments, about 1399 mg to about 1401 mg of the free acid form of the compound of Formula Ia, is administered to a patient in need thereof, once or twice every month. In some embodiments, about 1400 mg of the free acid form of the compound of Formula Ia, is administered to a patient in need thereof, once or twice every month.

In some embodiments, about 500 mg to about 8000 mg of the free acid form of the compound of Formula Ib, is administered to a patient in need thereof, once or twice every month. In some embodiments, about 1000 mg to about 1500 mg of the free acid form of the compound of Formula Ib, is administered to a patient in need thereof, once or twice every month. In some embodiments, about 1300 mg to about 1500 mg of the free acid form of the compound of Formula Ib, is administered to a patient in need thereof, once or twice every month. In some embodiments, about 1350 mg to about 1450 mg of the free acid form of the compound of Formula Ib, is administered to a patient in need thereof, once or twice every month. In some embodiments, about 1375 mg to about 1425 mg of the free acid form of the compound of Formula Ib, is administered to a patient in need thereof, once or twice every month. In some embodiments, about 1390 mg to about 1400 mg of the free acid form of the compound of Formula Ib, is administered to a patient in need thereof, once or twice every month. In some embodiments, about 1395 mg to about 1405 mg of the free acid form of the compound of Formula Ib, is administered to a patient in need thereof, once or twice every month. In some embodiments, about 1399 mg to about 1401 mg of the free acid form of the compound of Formula Ib, is administered to a patient in need thereof, once or twice every month. In some embodiments, about 1400 mg of the free acid form of the compound of Formula Ib, is administered to a patient in need thereof, once or twice every month.

In some embodiments, about 500 mg to about 8000 mg of an amorphous form of the free acid form of the compound of Formula Ia or Ib, is administered to a patient in need thereof, once or twice every month. In some embodiments, about 1000 mg to about 1500 mg of an amorphous form of the free acid form of the compound of Formula Ia or Ib, is administered to a patient in need thereof, once or twice every month. In some embodiments, about 1300 mg to about 1500 mg of an amorphous form of the free acid form of the compound of Formula Ia or Ib, is administered to a patient in need thereof, once or twice every month. In some embodiments, about 1350 mg to about 1450 mg of an amorphous form of the free acid form of the compound of Formula Ia or Ib, is administered to a patient in need thereof, once or twice every month. In some embodiments, about 1375 mg to about 1425 mg of an amorphous form of the free acid form of the compound of Formula Ia or Ib, is administered to a patient in need thereof, once or twice every month. In some embodiments, about 1390 mg to about 1400 mg of an amorphous form of the free acid form of the compound of Formula Ia or Ib, is administered to a patient in need thereof, once or twice every month. In some embodiments, about 1395 mg to about 1405 mg of an amorphous form of the free acid form of the compound of Formula Ia or Ib, is administered to a patient in need thereof, once or twice every month. In some embodiments, about 1399 mg to about 1401 mg of an amorphous form of the free acid form of the compound of Formula Ia or Ib, is administered to a patient in need thereof, once or twice every month. In some embodiments, about 1400 mg of an amorphous form of the free acid form of the compound of Formula Ia or Ib, is administered to a patient in need thereof, once or twice every month.

In some embodiments, about 500 mg to about 8000 mg of an amorphous form of the free acid form of the compound of Formula Ia, is administered to a patient in need thereof, once or twice every month. In some embodiments, about 1000 mg to about 1500 mg of an amorphous form of the free acid form of the compound of Formula Ia, is administered to a patient in need thereof, once or twice every month. In some embodiments, about 1300 mg to about 1500 mg of an amorphous form of the free acid form of the compound of Formula Ia, is administered to a patient in need thereof, once or twice every month. In some embodiments, about 1350 mg to about 1450 mg of an amorphous form of the free acid form of the compound of Formula Ia, is administered to a patient in need thereof, once or twice every month. In some embodiments, about 1375 mg to about 1425 mg of an amorphous form of the free acid form of the compound of Formula Ia, is administered to a patient in need thereof, once or twice every month. In some embodiments, about 1390 mg to about 1400 mg of an amorphous form of the free acid form of the compound of Formula Ia, is administered to a patient in need thereof, once or twice every month. In some embodiments, about 1395 mg to about 1405 mg of an amorphous form of the free acid form of the compound of Formula Ia, is administered to a patient in need thereof, once or twice every month. In some embodiments, about 1399 mg to about 1401 mg of an amorphous form of the free acid form of the compound of Formula Ia, is administered to a patient in need thereof, once or twice every month. In some embodiments, about 1400 mg of an amorphous form of the free acid form of the compound of Formula Ia, is administered to a patient in need thereof, once or twice every month.

In some embodiments, about 500 mg to about 8000 mg of an amorphous form of the free acid form of the compound of Formula Ib, is administered to a patient in need thereof, once or twice every month. In some embodiments, about 1000 mg to about 1500 mg of an amorphous form of the free acid form of the compound of Formula Ib, is administered to a patient in need thereof, once or twice every month. In some embodiments, about 1300 mg to about 1500 mg of an amorphous form of the free acid form of the compound of Formula Ib, is administered to a patient in need thereof, once or twice every month. In some embodiments, about 1350 mg to about 1450 mg of an amorphous form of the free acid form of the compound of Formula Ib, is administered to a patient in need thereof, once or twice every month. In some embodiments, about 1375 mg to about 1425 mg of an amorphous form of the free acid form of the compound of Formula Ib, is administered to a patient in need thereof, once or twice every month. In some embodiments, about 1390 mg to about 1400 mg of an amorphous form of the free acid form of the compound of Formula Ib, is administered to a patient in need thereof, once or twice every month. In some embodiments, about 1395 mg to about 1405 mg of an amorphous form of the free acid form of the compound of Formula Ib, is administered to a patient in need thereof, once or twice every month. In some embodiments, about 1399 mg to about 1401 mg of an amorphous form of the free acid form of the compound of Formula Ib, is administered to a patient in need thereof, once or twice every month. In some embodiments, about 1400 mg of an amorphous form of the free acid form of the compound of Formula Ib, is administered to a patient in need thereof, once or twice every month.

In some embodiments, about 2500 mg to about 3000 mg of the compound of Formula Ia or Ib, or a pharmaceutically acceptable salt thereof, is administered to a patient in need thereof, once or twice every month. In some embodiments, about 2700 mg to about 2900 mg of the compound of Formula Ia or Ib, or a pharmaceutically acceptable salt thereof, is administered to a patient in need thereof, once or twice every month. In some embodiments, about 2750 mg to about 2950 mg of the compound of Formula Ia or Ib, or a pharmaceutically acceptable salt thereof, is administered to a patient in need thereof, once or twice every month. In some embodiments, about 2775 mg to about 2925 mg of the compound of Formula Ia or Ib, or a pharmaceutically acceptable salt thereof, is administered to a patient in need thereof, once or twice every month. In some embodiments, about 2790 mg to about 2810 mg of the compound of Formula Ia or Ib, or a pharmaceutically acceptable salt thereof, is administered to a patient in need thereof, once or twice every month. In some embodiments, about 2795 mg to about 2805 mg of the compound of Formula Ia or Ib, or a pharmaceutically acceptable salt thereof, is administered to a patient in need thereof, once or twice every month. In some embodiments, about 2799 mg to about 2801 mg of the compound of Formula Ia or Ib, or a pharmaceutically acceptable salt thereof, is administered to a patient in need thereof, once or twice every month. In some embodiments, about 2800 mg of the compound of Formula Ia or Ib, or a pharmaceutically acceptable salt thereof, is administered to a patient in need thereof, once or twice every month.

In some embodiments, about 2500 mg to about 3000 of the free acid form of the compound of Formula Ia or Ib, is administered to a patient in need thereof, once or twice every month. In some embodiments, about 2700 mg to about 2900 mg of the free acid form of the compound of Formula Ia or Ib, is administered to a patient in need thereof, once or twice every month. In some embodiments, about 2750 mg to about 2950 mg of the free acid form of the compound of Formula Ia or Ib, is administered to a patient in need thereof, once or twice every month. In some embodiments, about 2775 mg to about 2925 mg of the free acid form of the compound of Formula Ia or Ib, is administered to a patient in need thereof, once or twice every month. In some embodiments, about 2790 mg to about 2810 mg of the free acid form of the compound of Formula Ia or Ib, is administered to a patient in need thereof, once or twice every month. In some embodiments, about 2795 mg to about 2805 mg of the free acid form of the compound of Formula Ia or Ib, is administered to a patient in need thereof, once or twice every month. In some embodiments, about 2799 mg to about 2801 mg of the free acid form of the compound of Formula Ia or Ib, is administered to a patient in need thereof, once or twice every month. In some embodiments, about 2800 mg of the free acid form of the compound of Formula Ia or Ib, is administered to a patient in need thereof, once or twice every month.

In some embodiments, about 2500 mg to about 3000 of the free acid form of the compound of Formula Ia, is administered to a patient in need thereof, once or twice every month. In some embodiments, about 2700 mg to about 2900 mg of the free acid form of the compound of Formula Ia, is administered to a patient in need thereof, once or twice every month. In some embodiments, about 2750 mg to about 2950 mg of the free acid form of the compound of Formula Ia, is administered to a patient in need thereof, once or twice every month. In some embodiments, about 2775 mg to about 2925 mg of the free acid form of the compound of Formula Ia, is administered to a patient in need thereof, once or twice every month. In some embodiments, about 2790 mg to about 2810 mg of the free acid form of the compound of Formula Ia, is administered to a patient in need thereof, once or twice every month. In some embodiments, about 2795 mg to about 2805 mg of the free acid form of the compound of Formula Ia, is administered to a patient in need thereof, once or twice every month. In some embodiments, about 2799 mg to about 2801 mg of the free acid form of the compound of Formula Ia, is administered to a patient in need thereof, once or twice every month. In some embodiments, about 2800 mg of the free acid form of the compound of Formula Ia, is administered to a patient in need thereof, once or twice every month.

In some embodiments, about 2500 mg to about 3000 of the free acid form of the compound of Formula Ib, is administered to a patient in need thereof, once or twice every month. In some embodiments, about 2700 mg to about 2900 mg of the free acid form of the compound of Formula Ib, is administered to a patient in need thereof, once or twice every month. In some embodiments, about 2750 mg to about 2950 mg of the free acid form of the compound of Formula Ib, is administered to a patient in need thereof, once or twice every month. In some embodiments, about 2775 mg to about 2925 mg of the free acid form of the compound of Formula Ib, is administered to a patient in need thereof, once or twice every month. In some embodiments, about 2790 mg to about 2810 mg of the free acid form of the compound of Formula Ib, is administered to a patient in need thereof, once or twice every month. In some embodiments, about 2795 mg to about 2805 mg of the free acid form of the compound of Formula Ib, is administered to a patient in need thereof, once or twice every month. In some embodiments, about 2799 mg to about 2801 mg of the free acid form of the compound of Formula Ib, is administered to a patient in need thereof, once or twice every month. In some embodiments, about 2800 mg of the free acid form of the compound of Formula Ib, is administered to a patient in need thereof, once or twice every month.

In some embodiments, about 2500 mg to about 3000 of an amorphous form of the free acid form of the compound of Formula Ia or Ib, is administered to a patient in need thereof, once or twice every month. In some embodiments, about 2700 mg to about 2900 mg of an amorphous form of the free acid form of the compound of Formula Ia or Ib, is administered to a patient in need thereof, once or twice every month. In some embodiments, about 2750 mg to about 2950 mg of an amorphous form of the free acid form of the compound of Formula Ia or

Ib, is administered to a patient in need thereof, once or twice every month. In some embodiments, about 2775 mg to about 2925 mg of an amorphous form of the free acid form of the compound of Formula Ia or Ib, is administered to a patient in need thereof, once or twice every month. In some embodiments, about 2790 mg to about 2810 mg of an amorphous form of the free acid form of the compound of Formula Ia or Ib, is administered to a patient in need thereof, once or twice every month. In some embodiments, about 2795 mg to about 2805 mg of an amorphous form of the free acid form of the compound of Formula Ia or Ib, is administered to a patient in need thereof, once or twice every month. In some embodiments, about 2799 mg to about 2801 mg of an amorphous form of the free acid form of the compound of Formula Ia or Ib, is administered to a patient in need thereof, once or twice every month. In some embodiments, about 2800 mg of an amorphous form of the free acid form of the compound of Formula Ia or Ib, is administered to a patient in need thereof, once or twice every month.

In some embodiments, about 2500 mg to about 3000 of an amorphous form of the free acid form of the compound of Formula Ia, is administered to a patient in need thereof, once or twice every month. In some embodiments, about 2700 mg to about 2900 mg of an amorphous form of the free acid form of the compound of Formula Ia, is administered to a patient in need thereof, once or twice every month. In some embodiments, about 2750 mg to about 2950 mg of an amorphous form of the free acid form of the compound of Formula Ia, is administered to a patient in need thereof, once or twice every month. In some embodiments, about 2775 mg to about 2925 mg of an amorphous form of the free acid form of the compound of Formula Ia, is administered to a patient in need thereof, once or twice every month. In some embodiments, about 2790 mg to about 2810 mg of an amorphous form of the free acid form of the compound of Formula Ia, is administered to a patient in need thereof, once or twice every month. In some embodiments, about 2795 mg to about 2805 mg of an amorphous form of the free acid form of the compound of Formula Ia, is administered to a patient in need thereof, once or twice every month. In some embodiments, about 2799 mg to about 2801 mg of an amorphous form of the free acid form of the compound of Formula Ia, is administered to a patient in need thereof, once or twice every month. In some embodiments, about 2800 mg of an amorphous form of the free acid form of the compound of Formula Ia, is administered to a patient in need thereof, once or twice every month.

In some embodiments, about 2500 mg to about 3000 of an amorphous form of the free acid form of the compound of Formula Ib, is administered to a patient in need thereof, once or twice every month. In some embodiments, about 2700 mg to about 2900 mg of an amorphous form of the free acid form of the compound of Formula Ib, is administered to a patient in need thereof, once or twice every month. In some embodiments, about 2750 mg to about 2950 mg of an amorphous form of the free acid form of the compound of Formula Ib, is administered to a patient in need thereof, once or twice every month. In some embodiments, about 2775 mg to about 2925 mg of an amorphous form of the free acid form of the compound of Formula Ib, is administered to a patient in need thereof, once or twice every month. In some embodiments, about 2790 mg to about 2810 mg of an amorphous form of the free acid form of the compound of Formula Ib, is administered to a patient in need thereof, once or twice every month. In some embodiments, about 2795 mg to about 2805 mg of an amorphous form of the free acid form of the compound of Formula Ib, is administered to a patient in need thereof, once or twice every month. In some embodiments, about 2799 mg to about 2801 mg of an amorphous form of the free acid form of the compound of Formula Ib, is administered to a patient in need thereof, once or twice every month. In some embodiments, about 2800 mg of an amorphous form of the free acid form of the compound of Formula Ib, is administered to a patient in need thereof, once or twice every month.

In some embodiments, about 6000 mg to about 8000 mg of the compound of Formula Ia or Ib, or a pharmaceutically acceptable salt thereof, is administered to a patient in need thereof, once or twice every month. In some embodiments, about 6500 mg to about 8000 mg of the compound of Formula Ia or Ib, or a pharmaceutically acceptable salt thereof, is administered to a patient in need thereof, once or twice every month. In some embodiments, about 6500 mg to about 7500 mg of the compound of Formula Ia or Ib, or a pharmaceutically acceptable salt thereof, is administered to a patient in need thereof, once or twice every month. In some embodiments, about 6900 mg to about 7100 mg of the compound of Formula Ia or Ib, or a pharmaceutically acceptable salt thereof, is administered to a patient in need thereof, once or twice every month. In some embodiments, about 6925 mg to about 7025 mg of the compound of Formula Ia or Ib, or a pharmaceutically acceptable salt thereof, is administered to a patient in need thereof, once or twice every month. In some embodiments, about 6990 mg to about 7010 mg of the compound of Formula Ia or Ib, or a pharmaceutically acceptable salt thereof, is administered to a patient in need thereof, once or twice every month. In some embodiments, about 6995 mg to about 7005 mg of the compound of Formula Ia or Ib, or a pharmaceutically acceptable salt thereof, is administered to a patient in need thereof, once or twice every month. In some embodiments, about 6999 mg to about 7001 mg of the compound of Formula Ia or Ib, or a pharmaceutically acceptable salt thereof, is administered to a patient in need thereof, once or twice every month. In some embodiments, about 7000 mg of the compound of Formula Ia or

Ib, or a pharmaceutically acceptable salt thereof, is administered to a patient in need thereof, once or twice every month.

In some embodiments, about 6000 mg to about 8000 mg of the free acid form of the compound of Formula Ia or Ib, is administered to a patient in need thereof, once or twice every month. In some embodiments, about 6500 mg to about 8000 mg of the free acid form of the compound of Formula Ia or Ib, is administered to a patient in need thereof, once or twice every month. In some embodiments, about 6500 mg to about 7500 mg of the free acid form of the compound of Formula Ia or Ib, is administered to a patient in need thereof, once or twice every month. In some embodiments, about 6900 mg to about 7100 mg of the free acid form of the compound of Formula Ia or Ib, is administered to a patient in need thereof, once or twice every month. In some embodiments, about 6925 mg to about 7025 mg of the free acid form of the compound of Formula Ia or Ib, is administered to a patient in need thereof, once or twice every month. In some embodiments, about 6990 mg to about 7010 mg of the free acid form of the compound of Formula Ia or Ib, is administered to a patient in need thereof, once or twice every month. In some embodiments, about 6995 mg to about 7005 mg of the free acid form of the compound of Formula Ia or Ib, is administered to a patient in need thereof, once or twice every month. In some embodiments, about 6999 mg to about 7001 mg of the free acid form of the compound of Formula Ia or Ib, is administered to a patient in need thereof, once or twice every month. In some embodiments, about 7000 mg of the free acid form of the compound of Formula Ia or Ib, is administered to a patient in need thereof, once or twice every month.

In some embodiments, about 6000 mg to about 8000 mg of the free acid form of the compound of Formula Ia, is administered to a patient in need thereof, once or twice every month. In some embodiments, about 6500 mg to about 8000 mg of the free acid form of the compound of Formula Ia, is administered to a patient in need thereof, once or twice every month. In some embodiments, about 6500 mg to about 7500 mg of the free acid form of the compound of Formula Ia, is administered to a patient in need thereof, once or twice every month. In some embodiments, about 6900 mg to about 7100 mg of the free acid form of the compound of Formula Ia, is administered to a patient in need thereof, once or twice every month. In some embodiments, about 6925 mg to about 7025 mg of the free acid form of the compound of Formula Ia, is administered to a patient in need thereof, once or twice every month. In some embodiments, about 6990 mg to about 7010 mg of the free acid form of the compound of Formula Ia, is administered to a patient in need thereof, once or twice every month. In some embodiments, about 6995 mg to about 7005 mg of the free acid form of the compound of Formula Ia, is administered to a patient in need thereof, once or twice every month. In some embodiments, about 6999 mg to about 7001 mg of the free acid form of the compound of Formula Ia, is administered to a patient in need thereof, once or twice every month. In some embodiments, about 7000 mg of the free acid form of the compound of Formula Ia, is administered to a patient in need thereof, once or twice every month.

In some embodiments, about 6000 mg to about 8000 mg of the free acid form of the compound of Formula Ib, is administered to a patient in need thereof, once or twice every month. In some embodiments, about 6500 mg to about 8000 mg of the free acid form of the compound of Formula Ib, is administered to a patient in need thereof, once or twice every month. In some embodiments, about 6500 mg to about 7500 mg of the free acid form of the compound of Formula Ib, is administered to a patient in need thereof, once or twice every month. In some embodiments, about 6900 mg to about 7100 mg of the free acid form of the compound of Formula Ib, is administered to a patient in need thereof, once or twice every month. In some embodiments, about 6925 mg to about 7025 mg of the free acid form of the compound of Formula Ib, is administered to a patient in need thereof, once or twice every month. In some embodiments, about 6990 mg to about 7010 mg of the free acid form of the compound of Formula Ib, is administered to a patient in need thereof, once or twice every month. In some embodiments, about 6995 mg to about 7005 mg of the free acid form of the compound of Formula Ib, is administered to a patient in need thereof, once or twice every month. In some embodiments, about 6999 mg to about 7001 mg of the free acid form of the compound of Formula Ib, is administered to a patient in need thereof, once or twice every month. In some embodiments, about 7000 mg of the free acid form of the compound of Formula Ib, is administered to a patient in need thereof, once or twice every month.

In some embodiments, about 6000 mg to about 8000 mg of an amorphous form of the free acid form of the compound of Formula Ia or Ib, is administered to a patient in need thereof, once or twice every month. In some embodiments, about 6500 mg to about 8000 mg of an amorphous form of the free acid form of the compound of Formula Ia or Ib, is administered to a patient in need thereof, once or twice every month. In some embodiments, about 6500 mg to about 7500 mg of an amorphous form of the free acid form of the compound of Formula Ia or Ib, is administered to a patient in need thereof, once or twice every month. In some embodiments, about 6900 mg to about 7100 mg of an amorphous form of the free acid form of the compound of Formula Ia or Ib, is administered to a patient in need thereof, once or twice every month. In some embodiments, about 6925 mg to about 7025 mg of an amorphous form of the free acid form of the compound of Formula Ia or Ib, is administered to a patient in need thereof, once or twice every month. In some embodiments, about 6990 mg to about 7010 mg of an amorphous form of the free acid form of the compound of Formula Ia or Ib, is administered to a patient in need thereof, once or twice every month. In some embodiments, about 6995 mg to about 7005 mg of an amorphous form of the free acid form of the compound of Formula Ia or Ib, is administered to a patient in need thereof, once or twice every month. In some embodiments, about 6999 mg to about 7001 mg of an amorphous form of the free acid form of the compound of Formula Ia or Ib, is administered to a patient in need thereof, once or twice every month. In some embodiments, about 7000 mg of an amorphous form of the free acid form of the compound of Formula Ia or Ib, is administered to a patient in need thereof, once or twice every month.

In some embodiments, about 6000 mg to about 8000 mg of an amorphous form of the free acid form of the compound of Formula Ia, is administered to a patient in need thereof, once or twice every month. In some embodiments, about 6500 mg to about 8000 mg of an amorphous form of the free acid form of the compound of Formula Ia, is administered to a patient in need thereof, once or twice every month. In some embodiments, about 6500 mg to about 7500 mg of an amorphous form of the free acid form of the compound of Formula Ia, is administered to a patient in need thereof, once or twice every month. In some embodiments, about 6900 mg to about 7100 mg of an amorphous form of the free acid form of the compound of Formula Ia, is administered to a patient in need thereof, once or twice every month. In some embodiments, about 6925 mg to about 7025 mg of an amorphous form of the free acid form of the compound of Formula Ia, is administered to a patient in need thereof, once or twice every month. In some embodiments, about 6990 mg to about 7010 mg of an amorphous form of the free acid form of the compound of Formula Ia, is administered to a patient in need thereof, once or twice every month. In some embodiments, about 6995 mg to about 7005 mg of an amorphous form of the free acid form of the compound of Formula Ia, is administered to a patient in need thereof, once or twice every month. In some embodiments, about 6999 mg to about 7001 mg of an amorphous form of the free acid form of the compound of Formula Ia, is administered to a patient in need thereof, once or twice every month. In some embodiments, about 7000 mg of an amorphous form of the free acid form of the compound of Formula Ia, is administered to a patient in need thereof, once or twice every month.

In some embodiments, about 6000 mg to about 8000 mg of an amorphous form of the free acid form of the compound of Formula Ib, is administered to a patient in need thereof, once or twice every month. In some embodiments, about 6500 mg to about 8000 mg of an amorphous form of the free acid form of the compound of Formula Ib, is administered to a patient in need thereof, once or twice every month. In some embodiments, about 6500 mg to about 7500 mg of an amorphous form of the free acid form of the compound of Formula Ib, is administered to a patient in need thereof, once or twice every month. In some embodiments, about 6900 mg to about 7100 mg of an amorphous form of the free acid form of the compound of Formula Ib, is administered to a patient in need thereof, once or twice every month. In some embodiments, about 6925 mg to about 7025 mg of an amorphous form of the free acid form of the compound of Formula Ib, is administered to a patient in need thereof, once or twice every month. In some embodiments, about 6990 mg to about 7010 mg of an amorphous form of the free acid form of the compound of Formula Ib, is administered to a patient in need thereof, once or twice every month. In some embodiments, about 6995 mg to about 7005 mg of an amorphous form of the free acid form of the compound of Formula Ib, is administered to a patient in need thereof, once or twice every month. In some embodiments, about 6999 mg to about 7001 mg of an amorphous form of the free acid form of the compound of Formula Ib, is administered to a patient in need thereof, once or twice every month. In some embodiments, about 7000 mg of an amorphous form of the free acid form of the compound of Formula Ib, is administered to a patient in need thereof, once or twice every month.

In some embodiments, the compound of Formula Ia or Ib, or a pharmaceutically acceptable salt thereof, is administered once every month. In some embodiments, the free acid form of the compound of Formula Ia or Ib, is administered once every month. In some embodiments, the free acid form of the compound of Formula Ia, is administered once every month. In some embodiments, the free acid form of the compound of Formula Ib, is administered once every month. In some embodiments, an amorphous form of the free acid form of the compound of Formula Ia or Ib, is administered once every month. In some embodiments, an amorphous form of the free acid form of the compound of Formula Ia, is administered once every month. In some embodiments, an amorphous form of the free acid form of the compound of Formula Ib, is administered once every month.

In some embodiments, the compound of Formula Ia or Ib, or a pharmaceutically acceptable salt thereof, is administered twice every month. In some embodiments, the free acid form of the compound of Formula Ia or Ib, is administered twice every month. In some embodiments, the free acid form of the compound of Formula Ia, is administered twice every month. In some embodiments, the free acid form of the compound of Formula Ib, is administered twice every month. In some embodiments, an amorphous form of the free acid form of the compound of Formula Ia or Ib, is administered twice every month. In some embodiments, an amorphous form of the free acid form of the compound of Formula Ia, is administered twice every month. In some embodiments, an amorphous form of the free acid form of the compound of Formula Ib, is administered twice every month.

In some embodiments, the methods provided herein comprise administering to the patient one or more of the tablets provided herein (e.g., administration of one tablet, two tablets, three tablets, and the like), once every month.

In some embodiments, the methods provided herein comprise administering to the patient one tablet provided herein, once every month. In some embodiments, the methods provided herein comprise administering to the patient one tablet provided herein, twice every month.

In some embodiments, the methods provided herein comprise administering to the patient two tablets provided herein, once every month. In some embodiments, the methods provided herein comprise administering to the patient two tablets provided herein, twice every month.

In some embodiments, the methods provided herein comprise administering to the patient three tablets provided herein, once every month. In some embodiments, the methods provided herein comprise administering to the patient three tablets provided herein, twice every month.

In some embodiments, the methods provided herein comprise administering to the patient four tablets provided herein, once every month. In some embodiments, the methods provided herein comprise administering to the patient four tablets provided herein, twice every month.

In some embodiments, the tablets provided herein are administered as a monotherapy.

In some embodiments, the methods provided herein further comprise administering to the patient a P-glycoprotein (PGP) inhibitor. In some embodiments, the P-glycoprotein (PGP) inhibitor provided herein is selected from verapamil, dexverapamil, cyclosporine, zosuquidar, laniquidar, elacridar, tariquidar, and encequidar.

In some embodiments, the PGP inhibitor is a compound of Formula II:

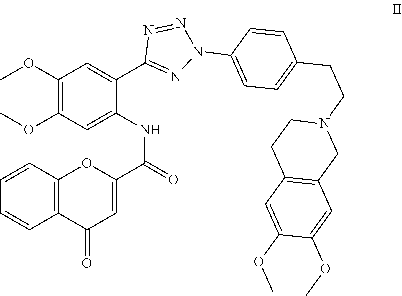 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]

or a pharmaceutically acceptable salt thereof. The compound of Formula II may also be referred to as encequidar (or ENC) or N-(2-(2-(4-(2-(6,7-dimethoxy-3,4-dihydroisoquinolin-2 (1H)-yl)ethyl)phenyl)-2H-tetrazol-5-yl)-4,5-dimethoxyphenyl)-4-oxo-4H-chromene-2-carboxamide. In some embodiments, the PGP inhibitor is a pharmaceutically acceptable salt of encequidar. In some embodiments, the PGP inhibitor is a mesylate salt of encequidar.

Increased intestinal expression of P-glycoprotein can reduce the absorption of drugs that are substrates for P-glycoprotein. PGP inhibitors may therefore be useful to modulate the PK properties of HIV capsid inhibitors (i.e., compounds of Formula Ia or Ib) disclosed herein. In some embodiments, the use of a PGP inhibitor in combination with a compound of Formula Ia or Ib, or a pharmaceutically acceptable salt thereof, disclosed herein can modulate (e.g. increase) the PK properties of the compound of Formula Ia or Ib, such as AUC, Cₘₐₓ, and oral bioavailability.

In some embodiments, the methods provided herein comprise administering to the subject about 1 mg to 4,000 mg of the PGP inhibitor, for example, about 2,000 to 4,000 mg, about 1 to 2,000 mg, about 1 to 1,000 mg, about 1 to 500 mg, about 10 to 500 mg, about 20 to 500 mg, about 1 to 300 mg, about 10 to 300 mg, about 20 to 300 mg, about 1 to 200 mg, about 10 to 200 mg, about 20 to 200 mg, about 1 to 100 mg, about 10 to 100 mg, about 20 to 100 mg, about 50 to 300 mg, about 75 to 200 mg, or about 15 to 150 mg of the PGP inhibitor.

In some embodiments, the methods provided herein comprise administering to the subject about 1 mg to about 50 mg of the PGP inhibitor.

In some embodiments, the methods provided herein comprise administering to the subject about 10 mg to about 50 mg of the PGP inhibitor.

In some embodiments, the methods provided herein comprise administering to the subject about 10 mg to about 30 mg of the PGP inhibitor.

In some embodiments, the methods provided herein comprise administering to the subject about 10 mg to about 20 mg of the PGP inhibitor.

In some embodiments, the methods provided herein comprise administering to the subject about 14 mg to about 16 mg of the PGP inhibitor.

In some embodiments, the methods provided herein comprise administering to the subject about 15 mg of the PGP inhibitor.

In some embodiments, the methods provided herein comprise administering to the subject about 1 mg to 4,000 mg of the compound of Formula II, or a pharmaceutically acceptable salt thereof, for example, about 2,000 to 4,000 mg, about 1 to 2,000 mg, about 1 to 1,000 mg, about 1 to 500 mg, about 10 to 500 mg, about 20 to 500 mg, about 1 to 300 mg, about 10 to 300 mg, about 20 to 300 mg, about 1 to 200 mg, about 10 to 200 mg, about 20 to 200 mg, about 1 to 100 mg, about 10 to 100 mg, about 20 to 100 mg, about 50 to 300 mg, about 75 to 200 mg, or about 15 to 150 mg of the compound of Formula II, or a pharmaceutically acceptable salt thereof.

In some embodiments, the methods provided herein comprise administering to the subject about 1 mg to about 50 mg of the compound of Formula II, or a pharmaceutically acceptable salt thereof. In some embodiments, the methods provided herein comprise administering to the subject about 10 mg to about 50 mg of the compound of Formula II, or a pharmaceutically acceptable salt thereof. In some embodiments, the methods provided herein comprise administering to the subject about 10 mg to about 30 mg of the compound of Formula II, or a pharmaceutically acceptable salt thereof. In some embodiments, the methods provided herein comprise administering to the subject about 10 mg to about 25 mg of the compound of Formula II, or a pharmaceutically acceptable salt thereof. In some embodiments, the methods provided herein comprise administering to the subject about 15 mg to about 25 mg of the compound of Formula II, or a pharmaceutically acceptable salt thereof. In some embodiments, the methods provided herein comprise administering to the subject about 15 mg to about 20 mg of the compound of Formula II, or a pharmaceutically acceptable salt thereof. In some embodiments, the methods provided herein comprise administering to the subject about 10 mg to about 20 mg of the compound of Formula II, or a pharmaceutically acceptable salt thereof. In some embodiments, the methods provided herein comprise administering to the subject about 19 mg to about 21 mg of the compound of Formula II, or a pharmaceutically acceptable salt thereof. In some embodiments, the methods provided herein comprise administering to the subject about 20 mg of the compound of Formula II, or a pharmaceutically acceptable salt thereof. In some embodiments, the methods provided herein comprise administering to the subject about 14 mg to about 16 mg of the compound of Formula II, or a pharmaceutically acceptable salt thereof. In some embodiments, the methods provided herein comprise administering to the subject about 15 mg of the compound of Formula II, or a pharmaceutically acceptable salt thereof.

In some embodiments, the methods provided herein comprise administering to the subject about 1 mg to about 50 mg of a pharmaceutically acceptable salt of the compound of Formula II. In some embodiments, the methods provided herein comprise administering to the subject about 10 mg to about 50 mg of a pharmaceutically acceptable salt of the compound of Formula II. In some embodiments, the methods provided herein comprise administering to the subject about 10 mg to about 30 mg of a pharmaceutically acceptable salt of the compound of Formula II. In some embodiments, the methods provided herein comprise administering to the subject about 10 mg to about 25 mg of a pharmaceutically acceptable salt of the compound of Formula II. In some embodiments, the methods provided herein comprise administering to the subject about 15 mg to about 25 mg of a pharmaceutically acceptable salt of the compound of Formula II. In some embodiments, the methods provided herein comprise administering to the subject about 15 mg to about 20 mg of a pharmaceutically acceptable salt of the compound of Formula II. In some embodiments, the methods provided herein comprise administering to the subject about 10 mg to about 20 mg of a pharmaceutically acceptable salt of the compound of Formula II. In some embodiments, the methods provided herein comprise administering to the subject about 19 mg to about 21 mg of a pharmaceutically acceptable salt of the compound of Formula II. In some embodiments, the methods provided herein comprise administering to the subject about 20 mg of a pharmaceutically acceptable salt of the compound of Formula II. In some embodiments, the methods provided herein comprise administering to the subject about 14 mg to about 16 mg of a pharmaceutically acceptable salt of the compound of Formula II. In some embodiments, the methods provided herein comprise administering to the subject about 15 mg of a pharmaceutically acceptable salt of the compound of Formula II.

In some embodiments, the methods provided herein comprise administering to the subject about 1 mg to about 50 mg of a mesylate salt of the compound of Formula II. In some embodiments, the methods provided herein comprise administering to the subject about 10 mg to about 50 mg of a mesylate salt of the compound of Formula II. In some embodiments, the methods provided herein comprise administering to the subject about 10 mg to about 30 mg of a mesylate salt of the compound of Formula II. In some embodiments, the methods provided herein comprise administering to the subject about 10 mg to about 25 mg of a mesylate salt of the compound of Formula II. In some embodiments, the methods provided herein comprise administering to the subject about 15 mg to about 25 mg of a mesylate salt of the compound of Formula II. In some embodiments, the methods provided herein comprise administering to the subject about 15 mg to about 20 mg of a mesylate salt of the compound of Formula II. In some embodiments, the methods provided herein comprise administering to the subject about 10 mg to about 20 mg of a mesylate salt of the compound of Formula II. In some embodiments, the methods provided herein comprise administering to the subject about 19 mg to about 21 mg of a mesylate salt of the compound of Formula II. In some embodiments, the methods provided herein comprise administering to the subject about 20 mg of a mesylate salt of the compound of Formula II. In some embodiments, the methods provided herein comprise administering to the subject about 14 mg to about 16 mg of a mesylate salt of the compound of Formula II. In some embodiments, the methods provided herein comprise administering to the subject about 15 mg of a mesylate salt of the compound of Formula II.

In some embodiments, the compound of Formula II, or a pharmaceutically acceptable salt thereof, is administered in the form of a pharmaceutical composition. In some embodiments, the pharmaceutical composition (i.e. formulation) comprising the compound of Formula II, or a pharmaceutically acceptable salt thereof, is suitable for oral administration. In some embodiments, the pharmaceutical composition comprising the compound of Formula II, or a pharmaceutically acceptable salt thereof, is a suspension or solution.

In some embodiments, the pharmaceutical composition comprising the compound of Formula II, or a pharmaceutically acceptable salt thereof, is a solution. In some embodiments, the pharmaceutical composition comprising the compound of Formula II, or a pharmaceutically acceptable salt thereof, is an injectable solution. In some embodiments, the pharmaceutical composition comprising the compound of Formula II, or a pharmaceutically acceptable salt thereof, is a solution suitable for oral administration. In some embodiments, the pharmaceutical composition comprising the compound of Formula II, or a pharmaceutically acceptable salt thereof, is an injectable solution suitable for oral administration.

In some embodiments, the pharmaceutical composition comprising the compound of Formula II, or a pharmaceutically acceptable salt thereof, is a suspension. In some embodiments, the pharmaceutical composition comprising the compound of Formula II, or a pharmaceutically acceptable salt thereof, is an injectable suspension. In some embodiments, the pharmaceutical composition comprising the compound of Formula II, or a pharmaceutically acceptable salt thereof, is a suspension suitable for oral administration. In some embodiments, the pharmaceutical composition comprising the compound of Formula II, or a pharmaceutically acceptable salt thereof, is an injectable suspension suitable for oral administration.

In some embodiments, the suspension comprising the compound of Formula II, or a pharmaceutically acceptable salt thereof, comprises about 1 mg/mL to about 10 mg/mL of the compound of Formula II, or a pharmaceutically acceptable salt thereof, for example, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, or about 10 mg/mL of the compound of Formula II, or a pharmaceutically acceptable salt thereof.

In some embodiments, the suspension comprising the compound of Formula II, or a pharmaceutically acceptable salt thereof, comprises about 1 mg/mL to about 5 mg/mL of the compound of Formula II, or a pharmaceutically acceptable salt thereof.

In some embodiments, the suspension comprising the compound of Formula II, or a pharmaceutically acceptable salt thereof, comprises about 1 mg/mL to about 3 mg/mL of the compound of Formula II, or a pharmaceutically acceptable salt thereof.

In some embodiments, the suspension comprising the compound of Formula II, or a pharmaceutically acceptable salt thereof, comprises about 1.5 mg/mL to about 2.5 mg/mL of the compound of Formula II, or a pharmaceutically acceptable salt thereof.

In some embodiments, the suspension comprising the compound of Formula II, or a pharmaceutically acceptable salt thereof, comprises about 1.9 mg/mL to about 2.1 mg/ml of the compound of Formula II, or a pharmaceutically acceptable salt thereof.

In some embodiments, the suspension comprising the compound of Formula II, or a pharmaceutically acceptable salt thereof, comprises about 2 mg/mL of the compound of Formula II, or a pharmaceutically acceptable salt thereof.

In some embodiments, the suspension comprising the compound of Formula II, or a pharmaceutically acceptable salt thereof, comprises carboxymethyl cellulose.

In some embodiments, the suspension comprising the compound of Formula II, or a pharmaceutically acceptable salt thereof, comprises about 0.1 w/w % to about 2 w/w % carboxymethyl cellulose, for example, about 0.1 w/w %, about 0.2 w/w %, about 0.3 w/w %, about 0.4 w/w %, about 0.5 w/w %, about 0.6 w/w %, about 0.7 w/w %, about 0.8 w/w %, about 0.9 w/w %, about 1 w/w %, about 1.1 w/w %, about 1.2 w/w %, about 1.3 w/w %, about 1.4 w/w %, about 1.5 w/w %, about 1.6 w/w %, about 1.7 w/w %, about 1.8 w/w %, about 1.9 w/w %, or about 2 w/w % carboxymethyl cellulose.

In some embodiments, the suspension comprising the compound of Formula II, or a pharmaceutically acceptable salt thereof, comprises about 0.1 w/w % to about 1 w/w % carboxymethyl cellulose.

In some embodiments, the suspension comprising the compound of Formula II, or a pharmaceutically acceptable salt thereof, comprises about 0.4 w/w % to about 0.6 w/w % carboxymethyl cellulose.

In some embodiments, the suspension comprising the compound of Formula II, or a pharmaceutically acceptable salt thereof, comprises about 0.5 w/w % carboxymethyl cellulose.

In some embodiments, the suspension comprising the compound of Formula II, or a pharmaceutically acceptable salt thereof, comprises about 98 w/w % to about 99.9 w/w % water, for example, about 98 w/w %, about 98.1 w/w %, about 98.2 w/w %, about 98.3 w/w %, about 98.4 w/w %, about 98.5 w/w %, about 98.6 w/w %, about 98.7 w/w %, about 98.8 w/w %, about 98.9 w/w %, about 99 w/w %, about 99.2 w/w %, about 99.3 w/w %, about 99.4 w/w %, about 99.5 w/w %, about 99.6 w/w %, about 99.7 w/w %, about 99.8 w/w %, or about 99.9 w/w % water.

In some embodiments, the suspension comprising the compound of Formula II, or a pharmaceutically acceptable salt thereof, comprises about 99 w/w % to about 99.9 w/w % water.

In some embodiments, the suspension comprising the compound of Formula II, or a pharmaceutically acceptable salt thereof, comprises about 99.5 w/w % water.

In some embodiments, the suspension comprising the compound of Formula II, or a pharmaceutically acceptable salt thereof, comprises carboxymethyl cellulose and water.

In some embodiments, the suspension comprising the compound of Formula II, or a pharmaceutically acceptable salt thereof, comprises about 1 mg/mL to about 10 mg/mL of the compound of Formula II, or a pharmaceutically acceptable salt thereof, about 0.1 w/w % to about 2 w/w % carboxymethyl cellulose, and about 98 w/w % to about 99.9 w/w % water.

In some embodiments, the suspension comprising the compound of Formula II, or a pharmaceutically acceptable salt thereof, comprises about 1 mg/mL to about 5 mg/mL of the compound of Formula II, or a pharmaceutically acceptable salt thereof, about 0.1 w/w % to about 1 w/w % carboxymethyl cellulose, and about 99 w/w % to about 99.9 w/w % water.

In some embodiments, the suspension comprising the compound of Formula II, or a pharmaceutically acceptable salt thereof, comprises about 2 mg/mL of the compound of Formula II, or a pharmaceutically acceptable salt thereof, about 0.5 w/w % carboxymethyl cellulose, and about 99.5 w/w % water.

In some embodiments, the solution comprising the compound of Formula II, or a pharmaceutically acceptable salt thereof, comprises Kolliphor HS-15, N-methylpyrrolidone, PEG 300, water, or any combination thereof.

In some embodiments, the solution comprising the compound of Formula II, or a pharmaceutically acceptable salt thereof, comprises Kolliphor HS-15.

In some embodiments, the solution comprising the compound of Formula II, or a pharmaceutically acceptable salt thereof, comprises about 5 w/w % to about 15 w/w % Kolliphor HS-15, for example, about 6 w/w %, about 7 w/w %, about 8 w/w %, about 9 w/w %, about 10 w/w %, about 11 w/w %, about 12 w/w %, about 13 w/w %, about 14 w/w %, or about 15 w/w % Kolliphor HS-15.

In some embodiments, the solution comprising the compound of Formula II, or a pharmaceutically acceptable salt thereof, comprises about 8 w/w % to about 12 w/w % Kolliphor HS-15.

In some embodiments, the solution comprising the compound of Formula II, or a pharmaceutically acceptable salt thereof, comprises about 9 w/w % to about 11 w/w % Kolliphor HS-15.

In some embodiments, the solution comprising the compound of Formula II, or a pharmaceutically acceptable salt thereof, comprises about 10 w/w % Kolliphor HS-15.

In some embodiments, the solution comprising the compound of Formula II, or a pharmaceutically acceptable salt thereof, comprises N-methylpyrrolidone.

In some embodiments, the solution comprising the compound of Formula II, or a pharmaceutically acceptable salt thereof, comprises about 1 w/w % to about 10 w/w % N-methylpyrrolidone, for example, about 1 w/w %, about 2 w/w %, about 3 w/w %, about 4 w/w %, about 5 w/w %, about 6 w/w %, about 7 w/w %, about 8 w/w %, about 9 w/w %, about 10 w/w % N-methylpyrrolidone.

In some embodiments, the solution comprising the compound of Formula II, or a pharmaceutically acceptable salt thereof, comprises about 3 w/w % to about 7 w/w % N-methylpyrrolidone.

In some embodiments, the solution comprising the compound of Formula II, or a pharmaceutically acceptable salt thereof, comprises about 4 w/w % to about 6 w/w % N-methylpyrrolidone.

In some embodiments, the solution comprising the compound of Formula II, or a pharmaceutically acceptable salt thereof, comprises about 5 w/w % N-methylpyrrolidone.

In some embodiments, the solution comprising the compound of Formula II, or a pharmaceutically acceptable salt thereof, comprises polyethylene glycol 300 (i.e., PEG 300).

In some embodiments, the solution comprising the compound of Formula II, or a pharmaceutically acceptable salt thereof, comprises about 20 w/w % to about 40 w/w % PEG 300, for example, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, about 30 w/w %, about 31 w/w %, about 32 w/w %, about 33 w/w %, about 34 w/w %, about 35 w/w %, about 36 w/w %, about 37 w/w %, about 38 w/w %, about 39 w/w %, or about 40 w/w % PEG 300.

In some embodiments, the solution comprising the compound of Formula II, or a pharmaceutically acceptable salt thereof, comprises about 25 w/w % to about 35 w/w % PEG 300.

In some embodiments, the solution comprising the compound of Formula II, or a pharmaceutically acceptable salt thereof, comprises about 29 w/w % to about 31 w/w % PEG 300.

In some embodiments, the solution comprising the compound of Formula II, or a pharmaceutically acceptable salt thereof, comprises about 30 w/w % PEG 300.

In some embodiments, the solution comprising the compound of Formula II, or a pharmaceutically acceptable salt thereof, comprises water.

In some embodiments, the solution comprising the compound of Formula II, or a pharmaceutically acceptable salt thereof, comprises about 45 w/w % to about 65 w/w % water, for example, about 45 w/w %, about 46 w/w %, about 47 w/w %, about 48 w/w %, about 49 w/w %, about 50 w/w %, about 51 w/w %, about 52 w/w %, about 53 w/w %, about 54 w/w %, about 55 w/w %, about 56 w/w %, about 57 w/w %, about 58 w/w %, about 59 w/w %, or about 60 w/w % water.

In some embodiments, the solution comprising the compound of Formula II, or a pharmaceutically acceptable salt thereof, comprises about 50 w/w % to about 60 w/w % water.

In some embodiments, the solution comprising the compound of Formula II, or a pharmaceutically acceptable salt thereof, comprises about 54 w/w % to about 56 w/w % water.

In some embodiments, the solution comprising the compound of Formula II, or a pharmaceutically acceptable salt thereof, comprises about 55 w/w % water.

In some embodiments, the solution comprising the compound of Formula II, or a pharmaceutically acceptable salt thereof, comprises Kolliphor HS-15, N-methylpyrrolidone, PEG 300, and water.

In some embodiments, the solution comprising the compound of Formula II, or a pharmaceutically acceptable salt thereof, comprises about 1 mg/mL to about 10 mg/mL of the compound of Formula II, or a pharmaceutically acceptable salt thereof, about 5 w/w % to about 15 w/w % Kolliphor HS-15, about 1 w/w % to about 10 w/w % N-methylpyrrolidone, about 20 w/w % to about 40 w/w % PEG 300, and about 45 w/w % to about 65 w/w % water.

In some embodiments, the solution comprising the compound of Formula II, or a pharmaceutically acceptable salt thereof, comprises about 1 mg/mL to about 5 mg/mL of the compound of Formula II, or a pharmaceutically acceptable salt thereof, about 8 w/w % to about 12 w/w % Kolliphor HS-15, about 3 w/w % to about 7 w/w % N-methylpyrrolidone, about 25 w/w % to about 35 w/w % PEG 300, and about 50 w/w % to about 60 w/w % water.

In some embodiments, the solution comprising the compound of Formula II, or a pharmaceutically acceptable salt thereof, comprises about 1.5 mg/mL to about 2.5 mg/mL of the compound of Formula II, or a pharmaceutically acceptable salt thereof, about 9 w/w % to about 11 w/w % Kolliphor HS-15, about 4 w/w % to about 6 w/w % N-methylpyrrolidone, about 29 w/w % to about 31 w/w % PEG 300, and about 54 w/w % to about 56 w/w % water.

In some embodiments, the solution comprising the compound of Formula II, or a pharmaceutically acceptable salt thereof, comprises about 2.0 mg/mL of the compound of Formula II, or a pharmaceutically acceptable salt thereof, about 10 w/w % Kolliphor HS-15, about 5 w/w % N-methylpyrrolidone, about 30 w/w % PEG 300, and about 55 w/w % water.

In some embodiments, the compounds provided herein are administered on a split dosing schedule (e.g., a compound of Formula Ia or Ib and the PGP inhibitor are administered in the morning and a compound of Formula Ia or Ib and/or the PGP inhibitor is administered in the evening).

In some embodiments, a compound provided herein (e.g., a compound of Formula Ia or Ib) and the PGP inhibitor provided herein (e.g., encequidar) are administered to the patient in the morning and in the evening.

In some embodiments, a compound provided herein (e.g., a compound of Formula Ia or Ib) and the PGP inhibitor provided herein (e.g., encequidar) are administered to the patient in the morning and a compound provided herein (e.g., a compound of Formula Ia or Ib) is administered to the patient in the evening.

In some embodiments, a compound provided herein (e.g., a compound of Formula Ia or Ib) and the PGP inhibitor provided herein (e.g., encequidar) are administered to the patient in the morning and the PGP inhibitor provided herein (e.g., encequidar) is administered to the patient in the evening.

In the regimens disclosed herein that involve administration in the morning and the evening, the morning and the evening may be between 8 hours apart and 16 hours apart, such as between 10 hours apart and 14 hours apart, or 12 hours apart. In some embodiments, a compound provided herein (e.g., a compound of Formula Ia or Ib) and the PGP inhibitor provided herein (e.g., encequidar) are administered to the patient in the morning and in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a compound provided herein (e.g., a compound of Formula Ia or Ib) and the PGP inhibitor provided herein (e.g., encequidar) are administered to the patient in the morning and a compound provided herein (e.g., a compound of Formula Ia or Ib) is administered to the patient in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a compound provided herein (e.g., a compound of Formula Ia or Ib) and the PGP inhibitor provided herein (e.g., encequidar) are administered to the patient in the morning and the PGP inhibitor provided herein (e.g., encequidar) is administered to the patient in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a compound provided herein (e.g., a compound of Formula Ia or Ib) and the PGP inhibitor (e.g., encequidar) are administered to the patient in the morning and in the evening, once per month.

In some embodiments, a compound provided herein (e.g., a compound of Formula Ia or Ib) and the PGP inhibitor (e.g., encequidar) are administered in the morning and a compound provided herein (e.g., a compound of Formula Ia or Ib) is administered to the patient in the evening, once per month.

In some embodiments, a compound provided herein (e.g., a compound of Formula Ia or Ib) and the PGP inhibitor (e.g., encequidar) are administered in the morning and the PGP inhibitor (e.g., encequidar) is administered to the patient in the evening, once per month.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and in the evening.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition provided herein (e.g., a tablet comprising a compound of Formula Ia or Ib) is administered to the patient in the evening.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition comprising the PGP inhibitor provided herein (e.g., a solution or suspension comprising a compound of Formula II) is administered to the patient in the evening.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition provided herein (e.g., a tablet comprising a compound of Formula Ia or Ib) is administered to the patient in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition comprising the PGP inhibitor provided herein (e.g., a solution or suspension comprising a compound of Formula II) is administered to the patient in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and in the evening, once per month.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition provided herein (e.g., a tablet comprising a compound of Formula Ia or Ib) is administered to the patient in the evening, once per month.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition comprising the PGP inhibitor provided herein (e.g., a solution or suspension comprising a compound of Formula II) is administered to the patient in the evening, once per month.

### EXAMPLES

The disclosure will be described in greater detail by way of specific examples. The following examples are offered for illustrative purposes, and are not intended to limit the disclosure in any manner. Those of skill in the art will readily recognize a variety of noncritical parameters which can be changed or modified to yield essentially the same results.

### Example 1. Tablet Formulations of Compound 1

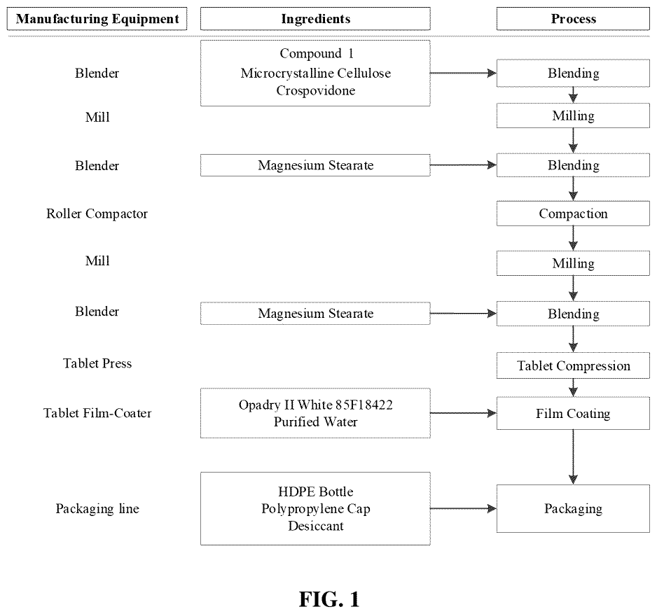

The following tablet formulations of Table 1 were prepared according to the manufacturing process flow diagram shown in FIG. 1. The tablets were prepared by dry granulation process, where the intragranular mixture was blended, milled, blended, compacted, milled, blended, and then compressed into tablets. Formulations 10-11 were coated with an outer film coating after being compressed into tablets.

TABLE 1
Tablet Formulations

| | w/w % | | | | | | |
|---|---|---|---|---|---|---|---|
| | Compound | | Microcrystalline | | Poloxamer | Magnesium | Dose |
| Formulation | 1 | Mannitol | Cellulose | Crospovidone | 407 | Stearate | (mg) |
| 2 | 40 | 27 | 27.5 | 4 | 0 | 1.5 | 306 |
| 3 | 40 | 26.75 | 26.75 | 5 | 0 | 1.5 | 350 |
| 4 | 60 | 16.75 | 16.75 | 5 | 0 | 1.5 | |
| 5 | 70 | 11.75 | 11.75 | 5 | 0 | 1.5 | |
| 6 | 60 | 14.25 | 14.25 | 10 | 0 | 1.5 | |
| 7 | 60 | 12.75 | 12.75 | 10 | 3 | 1.5 | |
| 8 | 60 | 0 | 28.5 | 10 | 0 | 1.5 | |
| 9 | 60 | 0 | 28 | 10 | 0 | 2 | |
| 10 | 60 | 0 | 28 | 10 | 0 | 2 | 300 |
| 11 | 60 | 0 | 28 | 10 | 0 | 2 | 700 |

### Example 2. In Vivo Dog Studies

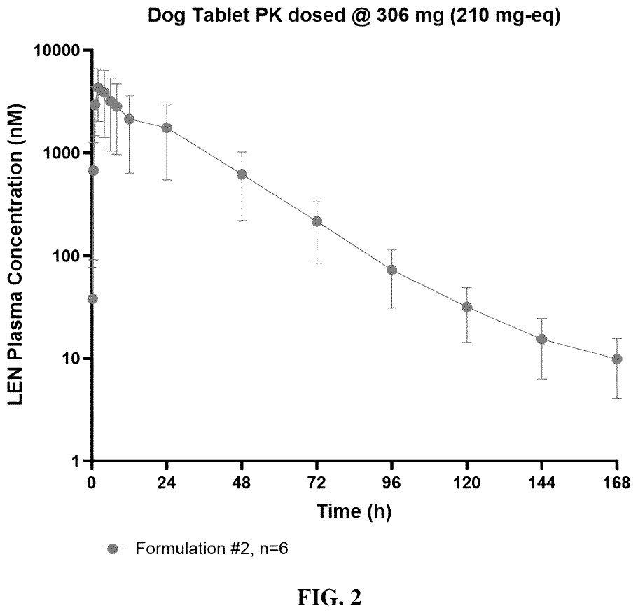

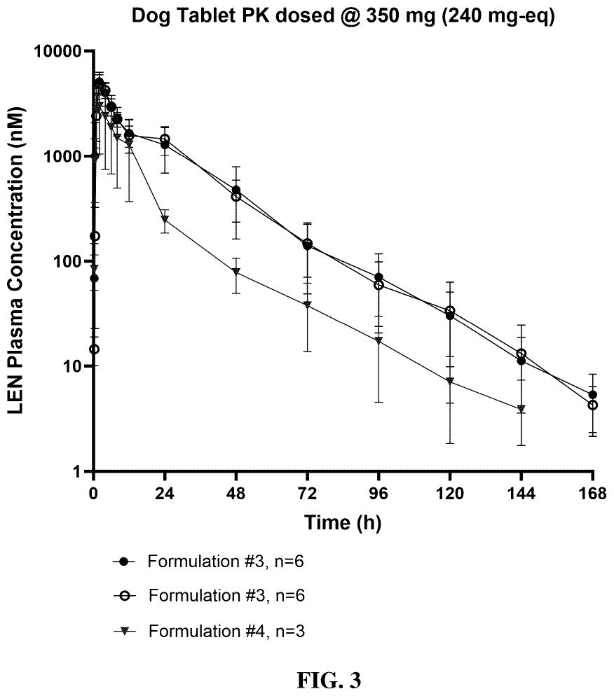

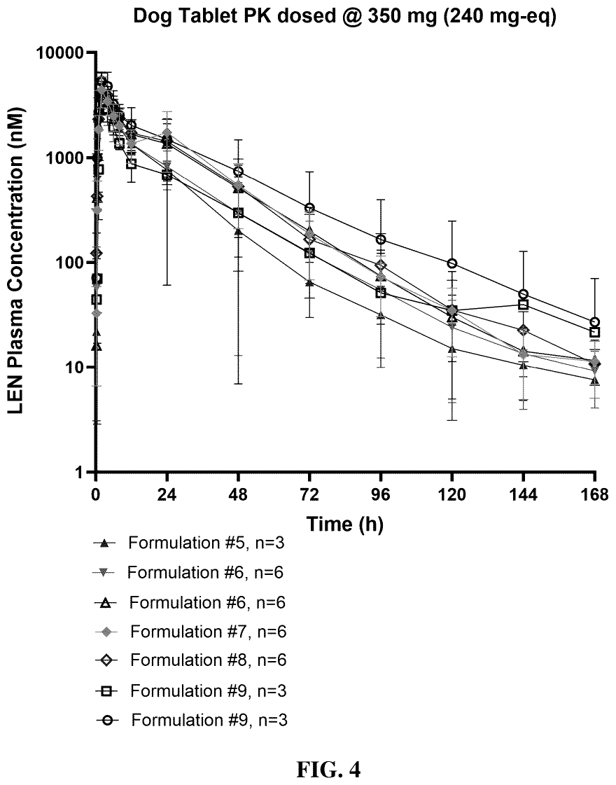

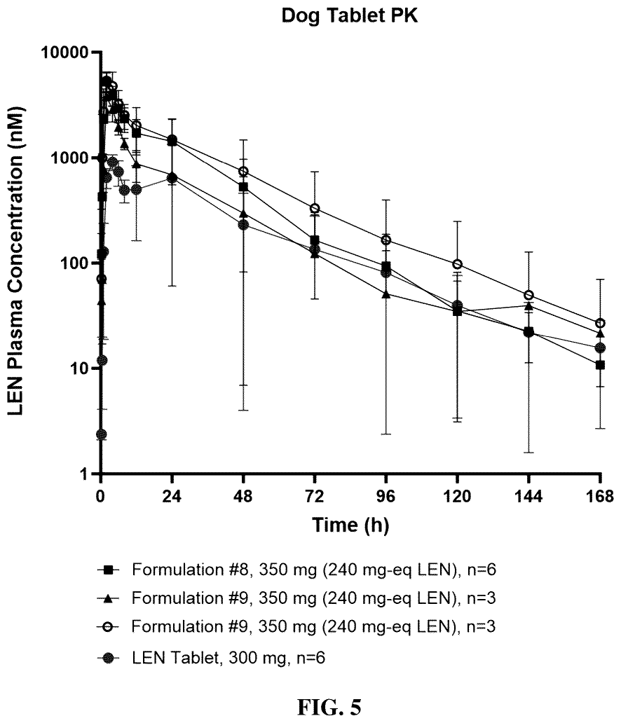

Tablets 2-9 of Example 1 were administered orally at 306 mg fixed dose to six beagle dogs or 350 mg fixed dose to either three or six beagle dogs and the pharmacokinetic (PK) profile was determined. A 306 mg-fixed dose of Compound 1 was equivalent to a 210 mg-fixed dose of lenacapavir (LEN). A 350 mg-fixed dose of Compound 1 was equivalent to a 240 mg-fixed dose of lenacapavir (LEN). For comparison, LEN spray dried dispersion (SDD) tablets (300 mg) were administered to six beagle dogs. Each LEN SDD tablet contained approximately 20.46 w/w % LEN sodium, 4.88 w/w % copovidone, 1.33 w/w % poloxamer 407, 42.55 w/w % mannitol, 21.28 w/w % microcrystalline cellulose, 8.0 w/w % croscarmellose sodium, and 1.5 w/w % magnesium stearate. Results of the dog studies are shown in FIGS. 2-5.

### Example 3. Encequidar Increases Oral Bioavailability and Decreases Intestinal Secretion Across Non-Clinical Species

Oral bioavailability of novel chemical entities can be limited by poor solubility, permeability, metabolic stability or transporter-mediated efflux. As one of the important factors, transporter-mediated efflux can greatly impact oral bioavailability (F %) and make achieving sufficient exposures a challenge (see e.g., Chan et al, European Journal of Pharmaceutical Sciences 2004, 21 (1), 25-51). Understanding the extent of transporter-mediated efflux in preclinical species such as rat, dog, and monkey can help with rational drug design to eliminate P-gp substrate affinity. For this reason, elacridar has been widely used as an efflux transporter inhibitor to help boost % F in preclinical species but suffers from poor formulatability (see e.g., Sawicki et al, Drug Delivery and Translational Research 2017, 7 (1), 125-131). Additionally, elacridar's systemic absorption can lead to drug-drug interactions (DDI) by inhibiting P-gp and BCRP at the bile-canalicular junction.

Recently, the ability of encequidar to inhibit P-gp was reported (see e.g., Chu et al, Pharm. Res. 2023; DOI: 10.1007/s11095-023-03563-4; and Smolinski et al, Journal of Medicinal Chemistry 2021, 64 (7), 3677-3693). Encequidar is a gut-restricted efflux transporter inhibitor that has been tested in rats and dogs with paclitaxel as a substrate (see e.g., Chu et al, Pharm. Res. 2023; DOI: 10.1007/s11095-023-03563-4). Encequidar is a useful tool to understand the influence of P-gp on % F limitations. Additionally, due to its gut-restricted properties, encequidar is a valuable tool for assessing the intestinal excretion clearance of molecules, a process mediated by efflux transporters such as P-gp and BCRP in the intestinal lumen. Because encequidar is not systemically absorbed, it selectively inhibits efflux transporters at the enterocyte level without affecting the bile-canalicular junction. This distinction enables encequidar to differentiate between intestinal secretion and biliary elimination, both influenced by systemic P-gp-mediated efflux.

This example describes the inhibition of encequidar in vitro against common transporters and cytochrome P450 enzymes to confirm its selectivity for human P-gp. In vivo experiments were then conducted in rats, dogs, and monkeys to demonstrate its impact on % F enhancement for P-gp substrates. To further illustrate encequidar's utility in distinguishing between intestinal excretion and biliary or hepatic metabolism-mediated clearances, IV studies on P-gp substrates known for intestinal excretion were conducted in rats and dogs.

### Formulation for In Vivo Assessment

The efflux probe substrate (test article) paclitaxel was formulated in 5% ethanol, 5% Kolliphor RH40, 20% PEG300, and 70% of 20% Kleptose in saline; digoxin was formulated in 5% NMP, 25% PEG300, and 70% of 20% Kleptose in saline; oral talinolol was formulated in 2% DMSO, 10% polyethylene glycol 300, and 88% sulfobutylether-β-cyclodextrin; IV talinolol and apixaban were co-formulated in 0.2% sodium phosphate, 30% 2-hydroxypropyl-β-cyclodextrin, and 69.8% water for injection. The efflux inhibitor encequidar was formulated in two forms-solution and suspension form: solution form was prepared in 5% NMP, 10% Kolliphor HS-15, 30% polyethylene glycol 300, and 55% water while the suspension form was prepared in 0.5% carboxymethylcellulose and 99.5% water. Elacridar HCl salt was formulated as a solution in 10% NMP, 20% PEG300, and 70% water.

### In Vivo Assessment in Sprague Dawley Rats

All animals were fasted overnight and had food returned at approximately 4 hours postdose. The inhibitor co-administered arm had a single dose of either encequidar (5 mg/kg, solution or suspension) or elacridar (10 mg/kg) 30 minutes prior to the test article. The no-inhibitor group was used as the control. Each group consisted of three male SD rats. The test article was either administered IV via lateral tail vein or PO via oral gavage. Whole blood was sampled up to 24 hours postdose following the test article administration and was treated with K₂EDTA, after which was centrifuged to obtain plasma. Feces was collected only for the IV group, and at intervals of 0 to 4, 4 to 8, and 8 to 24 hours postdose.

### In Vivo Assessment in Beagle Dogs and Cynomolgus Monkeys

Both IV and PO studies were carried out in beagle dog. Talinolol and apixaban were selected as the efflux probe substrates for IV administration at 0.33 mg/kg dose, and paclitaxel was selected for oral administration at 5 mg/kg dose. All animals were fasted overnight and had food returned at approximately 4 hour postdose. The test group was administered 5 mg/kg encequidar (solution) 30-min prior to the administration of efflux probe substrate, and no inhibitor group was used as the control. Each group consisted of three animals. For the IV group, in addition to plasma, feces were also collected up to 72 hours. The same study design was used for monkey in vivo assessment, except that only oral administration of talinolol at 1 mg/kg dose was conducted.

### Bioanalytical Method

200 μL acetonitrile (ACN) containing internal standard was added to a 50 μL aliquot of each plasma and diluted feces homogenate (homogenized using 50% ACN (3 mL/mg feces)), as well as matrix blanks containing reference standards. The precipitated proteins were removed by centrifugation and 100 μL of supernatant was transferred into a clean 96 deep-well plate and mixed with 200 μL of water. An aliquot of 5 μL was injected into a Thermo Fisher Vanquish LC coupled with an Exploris 240 HRMS system. The chromatographic separation was carried out on a Thermo Scientific Hypersil GOLD C18 column (50× 2.1 mm, 1.9 μm particle size). The mobile phase consisted of 0.1% formic acid in water as solvent A and 0.1% formic acid in acetonitrile as solvent B. At 1 mL/min flow rate, the elution program started at 20% B and held for 0.2 min; then linearly ramped to 95% B at 1.1 min and held for 0.2 min. Finally the gradient was re-equilibrated to 20% B for another 0.2 min. Full mass analysis was performed under H-ESI positive mode using the exact mass of each analyte at 30,000 resolution with 5 ppm mass tolerance. The mass spectrometry parameters were configured as 3500 V ion spray voltage, sheath gas 60, auxiliary gas 15, and sweep gas 2 (all arbitrary unit), 350° C. ion transfer tube temperature, and 400° C. vaporizer temperature. The LCMS raw data were processed using TraceFinder 5.1 and the noncompartment analysis was carried out using Dotmatics Ver 5.5-100976-s [2021 May 28].

### In Vitro Transporter Inhibition Assay

Overexpressing OATP 1B1 and 1B3 HEK 293 cells were seeded onto CellCoat Poly-D-Lysine coated 96-well black cell culture plates with clear bottoms at a density of 70,000 cells/well for WT and OATP1B1, or 100,000 cells/well for OATP1B3, respectively, and cultured to confluency overnight. All test compounds and reference inhibitors were serially diluted in DMSO to create spiking solutions at concentrations of 250-fold of final assay concentrations (final DMSO content 0.4%). Cells were preincubated with HBSS buffer containing test compounds for 15 minutes. After removing HBSS buffer, a 100 μL dose solution containing test article or reference inhibitor with probe substrate was added to the HEK293-OATP1B1, HEK-293-OATP1B3, and its associated WT cell plate, respectively. The co-incubation period of test compounds with the corresponding probe substrate for all plates was 5 minutes. Cells were washed three times with ice cold HBSS buffer and immediately extracted with 200 μL methanol:water (70:30; v: v) containing internal standard labetalol (30 nM). The 150 μL supernatants were evaporated to dryness on injection plates and reconstituted in 200 μL 80% water containing 20% acetonitrile and 0.1% formic acid for analysis by LC-MS/MS.

The transporter inhibition assay for human organic cation transporter (OCT) 2, organic anion transporter (OAT) 1 and 3, and multidrug and toxin extrusion protein (MATE) 1 and 2K, and breast cancer resistant protein (BCRP), and P-glycoprotein (P-gp) followed protocols from SOLVO Biotechnology (Budapest, Hungary). In brief, the in vitro test system was a polarized monolayer of MDCK-II cells grown on permeable supports. The MDCK-II cells were treated to express the transporter of interest or treated with a control vector. Unless stated otherwise, a stock solution was prepared with the test article in DMSO. All final assay solutions of probe substrate, reference inhibitor or test article contained 0.5% (v/v) DMSO. For OAT1, OAT3, OCT2, MATE1, and MATE2K, the pre-incubation time with inhibitors were 15 min and the uptake co-incubation time with probe substrate and inhibitors was 5 min. At the end of incubation, aspirated the uptake buffer and washed apical and basolateral side of the permeable support 4 times and added 60 μL cell extraction solution to the 96-well plate wells. After 15 min agitation on an orbital shaker at 120 rpm, collected 30 μL samples of the extract and added 200 μL scintillation fluid to extracted samples. The samples were counted on a 1450 Microbeta (Perkin-Elmer). For P-gp, the co-incubation time with probe substrate and inhibitors was 120 min; the co-incubation time with probe substrate and inhibitors was 60 min for BCRP. After incubation, aliquots (100 μL) were taken from the receptor chambers to determine the amount of translocated prazosin or digoxin, samples were analyzed by scintillation counting. The donor chambers were sampled before and after incubation to determine the initial concentration (CO) and recovery (R) of the probe substrate. Dose recovery samples were also taken from the helper plate to measure the actual initial concentration in the assay, the samples were quantified by LC-MS/MS.

### Results

### In Vitro Inhibition of Encequidar and Elacridar in HEK 293 or MDCK II Cells

As illustrated in Table 2, elacridar exhibited low IC₅₀ values (below 10 μM) for several transporters, including OATP1B1, OCT2, MATE1, MATE2K, and P-gP, indicating its broad in vitro inhibition profile. Conversely, encequidar showed no inhibition of OATP1B1, OATP1B3, OAT3, or BCRP up to the highest tested concentration (100 μM). However, encequidar did display potent inhibition of OCT2, MATE1, MATE2K, and P-gp, with IC₅₀ values in the single-digit micromolar range, and weak inhibition of OAT1 (IC₅₀=37 μM). This in vitro inhibition profile suggests that, unlike elacridar, encequidar preferentially inhibits P-gp without significantly affecting BCRP, highlighting its utility as a selective P-gp inhibitor.

Encequidar is not a substrate for the transporters OATP1B1 and OATP1B3. Additionally, it exhibited no inhibitory effect on any tested cytochrome P450 enzymes (CYP1A2, 2B6, 2C8, 2C9, 2C19, 2D6, 3A4), with IC₅₀ values exceeding 25 μM. Encequidar also did not act as a mechanism-based inhibitor of CYP3A4, as indicated by IC₅₀ values greater than 25 μM in the presence and absence of NADPH. Encequidar demonstrated no inhibition of UGT1A1 (IC₅₀>20 μM), further confirming its limited impact on major metabolic enzymes.

Permeability assessments showed that encequidar was undetectable in the basolateral compartments of both Caco-2 and MDCK systems, indicating low passive permeability. This low permeability supports its classification as a gut-restricted inhibitor, allowing selective inhibition of intestinal P-gp without systemic inhibitory effects. This property is advantageous for differentiating intestinal secretion from biliary elimination pathways, making encequidar a useful tool for investigating transporter-mediated intestinal secretion.

TABLE 2
Encequidar and elacridar in vitro transporter inhibition.

| | |
|---|---|
| Probe | IC₅₀ (μM) |

| Transporter | substrate | Elacridar | Encequidar |
|---|---|---|---|
| OATP1B1 | Pravastatin | 3.2 | NIO up to 100 |
| | | | μM |
| OATP1B3 | Pravastatin | 44 | NIO up to 100 |
| | | | μM |
| OAT1 | Tenofovir | 27 | 37 |
| OAT3 | MTX | 55 | NIO up to 100 |
| | | | μM |
| OCT2 | Metformin | 0.62 | 0.25 |
| MATE1 | Metformin | 0.75 | 0.58 |
| MATE2K | Metformin | 0.17 | 0.13 |
| P-gp | Digoxin | 0.19 | 5.7 |
| BCRP | Prazosin | 6.8 | NIO up to 100 |
| | | | μM |

NIO: no inhibition observed; MTX: methotrexate.

### Pharmacokinetics of Encequidar in Non-Clinical Species

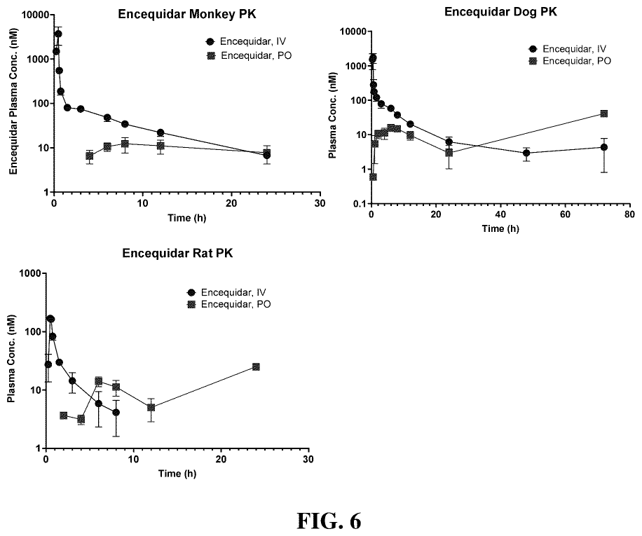

The PK profile of encequidar was investigated in rats, dogs, and monkeys, with the plasma PK profiles shown in FIG. 6 and summarized PK parameters in Tables 3A-3B. Encequidar exhibited moderate to high plasma clearance across species, with hepatic extraction ratio (ER) ranging from 0.48 to >1, indicating substantial systemic elimination. Clearance was highest in rats, exceeding hepatic blood flow, likely due to the observed instability of encequidar in rat plasma (t½=76 min), suggesting significant non-hepatic elimination pathways in this species.

The volume of distribution at steady state (Vₛₛ) was high across species, ranging from 3.2 L/kg in cynos to 15.4 L/kg in rats, indicative of extensive tissue distribution. The terminal half-life (t½) varied markedly, from 2.1 hours in rats to 22.8 hours in dogs, highlighting species-specific differences in encequidar disposition. Encequidar demonstrated a biphasic PK profile characterized by rapid distribution followed by a slower elimination phase.

The oral bioavailability (F %) of encequidar was low in all species (<10%), consistent with its low passive permeability observed in Caco-2 and MDCK transwell systems. This limited oral absorption supports encequidar's utility as a gut-restricted P-gp inhibitor without significant systemic exposure, allowing selective inhibition of intestinal efflux without off-target effects.

In vitro stability assessment indicated that encequidar was stable in human, beagle dog, cynomolgus, and rhesus monkey plasma but degraded rapidly in rat plasma. Plasma protein binding was extensive in humans, cynomolgus monkeys, and rats, with a free fraction of less than 0.1% in plasma and human liver microsomes. Despite high plasma protein binding and low to moderate predicted hepatocytes extraction ratios (15-44% of hepatic blood flow across species), encequidar displayed moderate to high in vivo clearance. The elevated clearance in rats, exceeding hepatic blood flow, aligns with encequidar's plasma instability in this species.

In Tables 3A-3B, for IV administration, parameters include clearance (CL), predicted hepatic clearance (Pred Hep CL), volume of distribution at steady state (Vₛₛ), terminal half-life (t_{1/2}), and mean residence time (MRT). For PO administration, parameters include maximum plasma concentration (Cₘₐₓ), time to reach maximum plasma concentration (Tₘₐₓ), and oral bioavailability (% F).

TABLE 3A
Encequidar IV PK parameters

| | Rat | Dog | Monkey |
|---|---|---|---|
| CL, L/h/kg | 7.88 ± | 0.869 ± | 0.781 ± |
| (ER) | 1.54 (>1) | 0.242 (0.54) | 0.126 (0.49) |
| Pred Hep CL, | 1.76 | 0.72 | 0.25 |
| L/h/kg | | | |
| Vₛₛ, L/kg | 15.4 ± 1.99 | 7.73 ± 1.22 | 3.19 ± 0.749 |
| t_{1/2}, h | 2.09 ± 0.46 | 22.8 | 6.94 ± 0.748 |
| MRT, h | 2.04 ± 0.639 | 9.14 ± 1.62 | 4.05 ± 0.372 |

TABLE 3B
Encequidar PO PK parameters

| | Rat | Dog | Monkey |
|---|---|---|---|
| Cₘₐₓ, nM | 17.9 ± 6.48 | 16.9 ± 2.11 | 12.5 ± 4.38 |
| Tₘₐₓ, h | 12.7 ± 9.87 | 6.67 ± 1.15 | 7.33 ± 1.15 |
| % F | 8.23 ± 4 | 1.44 ± 0.225 | 1.03 ± 0.534 |

### Encequidar Boosts Oral Exposures of P-Gp Substrates Across Species

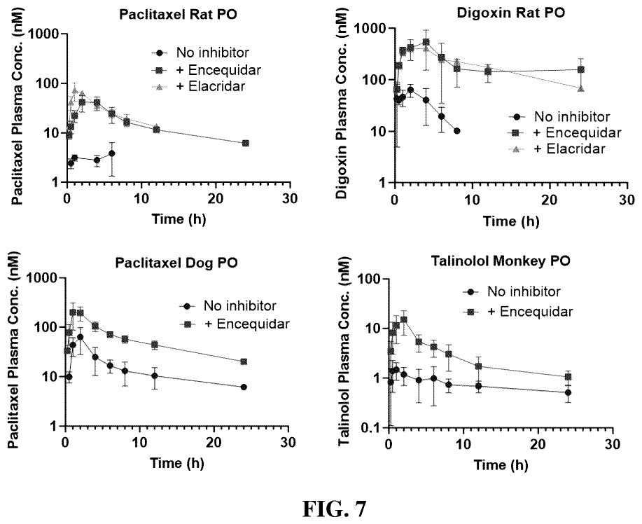

The pharmacokinetics of orally administered digoxin and paclitaxel in rats, with or without efflux inhibitors, are summarized in Table 4 and FIG. 7. Co-administration of encequidar led to a 10-fold increase in Cₘₐₓ and 20-fold increase in AUCₗₐₛₜ for digoxin, while elacridar produced similar effects with a 7-fold and 18-fold increase, respectively. For paclitaxel, encequidar co-administration resulted in a 13-fold increase in Cₘₐₓ and a 20-fold increase in AUCₗₐₛₜ, with elacridar showing comparable effects. Thus, both encequidar and elacridar markedly enhanced the oral exposures of digoxin and paclitaxel in rats.

Further experiments tested encequidar in a suspension formulation with paclitaxel to assess formulation impact on efflux inhibition. Results showed that the suspension formulation of encequidar achieved similar exposure increases as the solution, confirming that the observed inhibition is intrinsic to encequidar itself and not formulation-dependent.

In dogs, oral co-administration of encequidar with paclitaxel increased AUCₗₐₛₜ approximately 5-fold and Cₘₐₓ 4-fold, demonstrating effective P-gp inhibition in the gastrointestinal tract. Similarly, co-administration with talinolol in monkeys increased AUCₗₐₛₜ by 5-fold and Cₘₐₓ by 10-fold, further indicating potent P-gp inhibition by encequidar in primates.

The PK profiles show that the elimination phase of the substrates in dogs and monkeys was unaffected by encequidar, suggesting the increased oral exposure is primarily due to enhanced bioavailability rather than altered systemic clearance. Furthermore, as indicated in the encequidar PK section, plasma concentrations of encequidar remained below 10 nM post-5 mg/kg administration in rats, dogs, and monkeys, minimizing the likelihood of systemic inhibition of drug-metabolizing enzymes or other transporters.

In Table 4, “NC” indicates values that could not be calculated due to the absence of a clear terminal phase, and “NA” indicates parameters not applicable due to lack of fecal sample collection.

TABLE 4
Pharmacokinetic parameters of digoxin, paclitaxel, and talinolol in rats, dogs,
and monkeys following oral (PO) administration with or without co-administration
of the P-gp inhibitors encequidar (5 mg/kg PO) or elacridar (10 mg/kg PO)

| | | | AUCₗₐₛₜ | | |
|---|---|---|---|---|---|
| Species | Substrate, Dose | Inhibitor, Dose | nM*h | Cₘₐₓ, nM | Tₘₐₓ, nM |
| Rat | Digoxin, 5 mg/kg | None | 258 ± 135 | 64 ± 18 | 1.4 ± 1.0 |
| | | Encequidar, 5 mg/kg | 5170 ± 1280 | 623 ± 331 | 3.3 ± 1.2 |
| | | Elacridar, 10 mg/kg | 4720 ± 963 | 447 ± 58 | 3.3 ± 1.2 |
| Rat | Paclitaxel, 5 mg/kg | None | 15.3 ± 4.82 | 4.02 ± 1.48 | 2.7 ± 2.9 |
| | | Encequidar, 5 mg/kg | 361 ± 89 | 52.4 ± 2.84 | 2.7 ± 1.2 |
| | | Elacridar, 10 mg/kg | 358 ± 87 | 78.9 ± 24.5 | 1.3 ± 0.6 |
| Dog | Paclitaxel, 5 mg/kg | None | 300 ± 211 | 62.7 ± 34.8 | 2 |
| | | Encequidar, 5 mg/kg | 1470 ± 276 | 245 ± 68.4 | 1.67 ± 0.58 |
| Monkey | Talinolol, 1 mg/kg | None | 18.3 ± 5.3 | 1.56 ± 0.58 | 0.67 ± 0.29 |
| | | Encequidar, 5 mg/kg | 83.7 ± 38.6 | 15.2 ± 7.92 | 2 ± 0 |

### Encequidar can Inhibit Intestinal Secretion in Rat and Dog

Paclitaxel was dosed IV (1 mpk) in the absence and presence of 5 mpk PO encequidar or 10 mpk elacridar and the amount of parent was quantified in the feces. Encequidar and elacridar reduced the amount of parent in the feces from 20% to less than 1% thereby proving that encequidar and elacridar can inhibit P-gp in the enterocytes thereby stopping intestinal secretion. Correspondingly, the IV clearance of paclitaxel went up by 18% in the presence of the P-gp inhibitors.

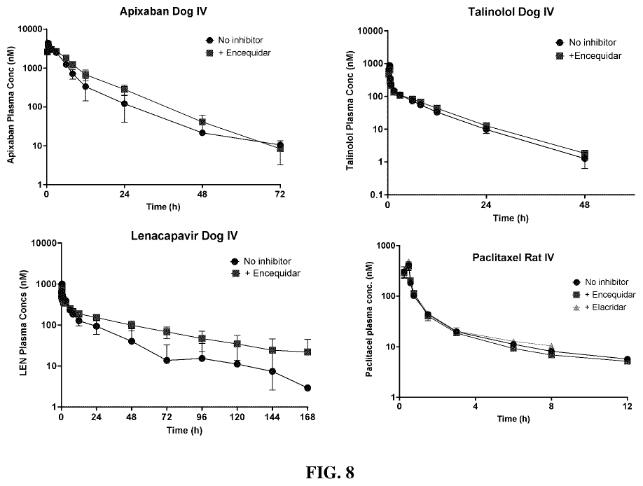

Encequidar is also capable of inhibiting intestinal secretion clearance and this was tested in dogs with apixaban, lenacapavir, and talinolol as compounds that undergo intestinal secretion. As shown in FIG. 8 and Table 5, following apixaban IV, approximately 31% was recovered in feces in unchanged form. This excretion in feces was reduced to 11% when co-administered with encequidar with a corresponding reduction in CL from 0.33 to 0.23 L/kg/h. Similarly, excretion of talinolol in feces was reduced from 10% to less than 1% with encequidar co-administration with a corresponding reduction in CL from 0.54 to 0.51 L/kg/h. With lenacapavir, the excretion in feces was reduced from 32 to 19% with a corresponding reduction in CL from 0.051 to 0.023 L/kg/h. Hence, in dogs, encequidar was capable of significantly reducing the extent of intestinal secretion. The parent found in feces upon coadministration of encequidar could be via biliary elimination, and hence encequidar is a useful tool to quantitatively estimate both intestinal secretion clearance and biliary clearance.

TABLE 5
Pharmacokinetic parameters of apixaban, talinolol, lenacapavir, and paclitaxel
in beagle dogs and Sprague-Dawley rats following IV administration, with or
without co-administration of the P-gp inhibitor encequidar (5 mg/kg PO)

| | | | | CL | Vₛₛ | Excretion |
|---|---|---|---|---|---|---|
| Species | Substrate | Inhibitor | t_{1/2} (h) | (L/hr/kg) | (L/kg) | in feces (%) |
| Dog | Apixaban | None | 6.9 ± 3.4 | 0.033 ± 0.008 | 0.21 ± 0.03 | 31 ± 10 |
| | | Encequidar | 10.1 ± 1.9 | 0.023 ± 0.004 | 0.22 ± 0.02 | 11 ± 3 |
| Dog | Talinolol | None | 7.6 ± 0.8 | 0.54 ± 0.06 | 3.88 ± 0.25 | 10 ± 3 |
| | | Encequidar | 7.6 ± 0.3 | 0.51 ± 0.05 | 4.43 ± 0.36 | 0.7 ± 0.2 |
| Dog | Lenacapavir | None | 15.9 ± 10.1 | 0.051 ± 0.019 | 0.90 ± 0.12 | 31.5 ± 9.0 |
| | | Encequidar | 45.1 ± 20.6 | 0.023 ± 0.008 | 1.34 ± 0.20 | 18.8 ± 4.1 |
| Rat | Paclitaxel | None | 4.0 ± 1.0 | 3.41 ± 0.81 | 6.5 ± 2.0 | 19.8 ± 6.8 |
| | | Encequidar | 6.5 ± 1.0 | 2.78 ± 0.12 | 12.4 ± 2.9 | 0.9 ± 0.7 |
| | | Elacridar | 5.2 ± 1.6 | 2.82 ± 0.47 | 7.5 ± 4.0 | 0.5 ± 0.3 |

### SUMMARY

### Encequidar is a Selective Human P-Gp Inhibitor

To understand the potency and selectivity of elacridar and encequidar, in vitro IC₅₀ of several common human efflux and uptake transporters using cells or membrane vesicles overexpressing these transporters were analyzed. Elacridar was found to potently inhibit multiple transporters, including OATP1B1, OCT2, MATE1, MATE2K, P-gp and BCRP with IC₅₀ values below 10 μM. Additionally, elacridar demonstrated moderate inhibition of OATP1B3, OAT1, and OAT3 with IC₅₀ values ranging from 27 to 55 μM. In contrast, encequidar showed no measurable inhibition of OATP1B1, OATP1B3, OAT3, and BCRP, and only moderate inhibition towards OAT1 (IC₅₀=37 μM).

In pharmacokinetic studies conducted in rats, dogs and monkeys, oral bioavailability of encequidar was observed to be less than 10%, with Cₘₐₓ values below 20 nM. Given encequidar's high plasma protein binding (>99.9% across species), and without being bound by theory, the estimated free circulating concentrations post-oral administration are likely to be in single to double-digit picomolar range, suggesting minimal potential for systemic inhibition. Furthermore, encequidar does not inhibit major cytochrome P450 enzymes and is not a time-dependent inhibitor of CYP3A4.

The in vitro transporter and enzyme inhibition data, combined with the in vivo PK profile, indicate that encequidar's P-gp inhibition is restricted to the gastrointestinal tract. Its selectivity for human P-gp over human BCRP makes it a useful tool for investigating P-gp-mediated efflux mechanism.

### Encequidar Shows High Clearance and Low Oral Bioavailability

Encequidar was administered to rats, dogs, and monkeys, and exhibited moderate to high clearance across species. In cryopreserved hepatocyte assays, predicted clearance was low to moderate, yet in vivo clearance remained moderate to high, despite encequidar's very high plasma protein binding (>99.9% in human, rat, dog and monkey). This discrepancy suggests that factors beyond hepatic metabolism, such as plasma instability, contribute to encequidar's clearance, especially in rats, where clearance exceeded hepatic blood flow, aligning with its observed instability in rat plasma.

In dogs and monkeys, encequidar displayed a biphasic pharmacokinetic profile with a long terminal half-life ranging from 7 to 23 hours. Following oral dosing at 10 mg/kg in solution, encequidar exposures were very low with bioavailability ranging from 1% to 8% across species and Cₘₐₓ values consistently below 20 nM. These low oral exposures correlate well with the low permeability determined from MDCK and Caco-2 where the permeability values were below the detection limit of 0.1×10⁻⁶ cm/s. The Fₐ×F_{g} values (fraction absorbed and fraction escaping gut metabolism) for dog and monkey were 2 and 3% respectively, indicating that both poor permeability and metabolic instability contribute to encequidar's limited oral exposures. These findings underscore encequidar's gut-restricted nature, suggesting that any pharmacokinetic impact or DDIs with encequidar as the perpetrator would be limited to the GI tract with minimal systemic effects.

### Encequidar Boosts Oral Bioavailability of P-Gp Substrates Across Preclinical Species

Administration of 5 mg/kg encequidar in a solution formulation increased Cₘₐₓ and AUC values of P-gp substrates by 5- to 20-fold across rat, dog and monkey. This study represents the first comprehensive report of encequidar's ability to enhance P-gp substrate exposures across multiple preclinical species, highlighting its utility as a pharmacokinetic booster. Given encequidar's gut-restricted nature, this enhancement in oral bioavailability is due to efflux transporter inhibition at enterocyte level, suggesting that encequidar has sufficient permeability to access enterocytes and inhibit the efflux transporters, yet lacks the permeability and metabolic stability necessary for significant systemic exposure.

Furthermore, comparable bioavailability improvements for paclitaxel were observed with both suspension and solution formulations of encequidar, indicating formulation flexibility. This ease of formulation, combined with its selective enterocyte-level action, makes encequidar an attractive tool for investigating drug disposition mechanisms.

### Encequidar is Capable of Inhibiting Intestinal Secretion in Rat and Dog

When paclitaxel was administered intravenously in rats, co-administration of oral elacridar reduced the fecal excretion of paclitaxel from 20% to 1% of the administered dose. This suggests that approximately 20% of paclitaxel could be eliminated via biliary elimination or intestinal secretion, both of which are inhibited by elacridar. However, when a similar 20% reduction of fecal excretion was observed with the co-administration of encequidar, this indicates that paclitaxel undergoes intestinal secretion in rats, instead of biliary elimination. Thus, encequidar serves as a useful tool not only for quantifying the extent of intestinal secretion, but also for differentiating between intestinal and biliary elimination pathways.

Further investigations into the role of encequidar in inhibiting intestinal secretion were conducted in dogs, where apixaban, talinolol, and lenacapavir were co-administered as an IV cassette at 0.33 mg/kg each. For apixaban, fecal excretion decreased from 31 to 11% with encequidar, suggesting approximately 20% intestinal secretion and 11% biliary elimination in dogs. This decrease in fecal excretion was consistent with a 30% reduction in IV clearance. For talinolol, fecal excretion decreased from 10% to 1%, indicating around 9% intestinal secretion and negligible biliary elimination. Correspondingly, IV clearance decreased by 6%, aligning with the reduction in intestinal secretion. For lenacapavir, fecal excretion decreased from 32% to 19%, indicating 13% intestinal secretion and 19% biliary elimination. Lenacapavir's IV clearance reduced by 54%, which exceeded the reduction in intestinal secretion.

In summary, this study demonstrates that encequidar acts as a gut-restricted efflux inhibitor in rats, dogs, and monkeys. Oral co-administration of encequidar PO significantly enhances the oral bioavailability of the efflux substrates and reduces their intestinal excretion into feces. This gut-specific inhibitory effect on intestinal secretion makes encequidar a valuable tool for assessing the contribution of intestinal excretion in the overall disposition of drug candidates in preclinical models, without the need for bile duct cannulation (BDC) studies.

### Example 4. Encequidar Vehicle Exhibits Inhibitory Effect in Compound 1

A 350 mg fixed dose of Compound 1 was orally administered to each animal in a tablet formulation containing 10% crospovidone, 1.5% magnesium stearate, and 28.5% microcrystalline cellulose.

Dog PO solution vehicle (2.0 mg/mL) composed of 10% Kolliphor HS-15, 5% NMP, 30% polyethylene glycol 300, 55% water for injection were prepared according to the following procedures. Animals were dosed at 2.5 mL/kg within four hours of preparing the solution vehicle. Study leg 1=administration of Compound 1 only; Study leg 2=administration of 2.0 mg/mL solution vehicle (no encequidar) followed by Compound 1 tablet.
Leg 1: All animals were administered a 0.1 mL/kg intramuscular injection of pentagastrin approximately 30 minutes (+3 minutes) prior to administration of Compound 1 tablet.
Leg 2: All animals were administered a 2.5 mL/kg oral dose of solution vehicle (no encequidar) composed of 10% Kolliphor HS-15, 5% NMP, 30% polyethylene glycol 300, 55% water for injection, immediately followed by a 0.1 mL/kg intramuscular injection of pentagastrin approximately 30 minutes (+3 minutes) prior to administration of Compound 1 tablet.

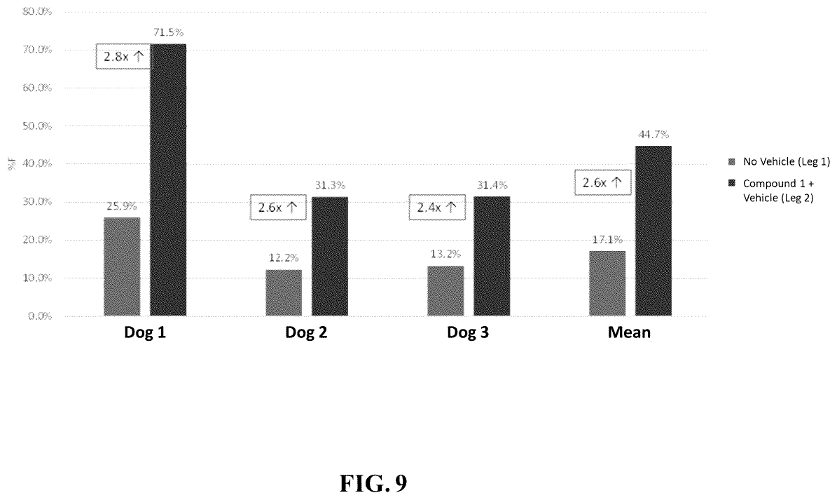

Table 6 shows PK parameters of lenacapavir after an oral dose at 350 mg fixed of Compound 1 in pentagastrin pretreated dogs, with and without solution vehicle (n=3). % F of lenacapavir was calculated using AUCinf 18400 nM·h from average of Dog IV at 1 mg/kg. As shown in FIG. 9, administration of Compound 1, 350 mg fixed PO+solution vehicle showed an average 2.6×% F lenacapavir improvement.

TABLE 6
PK parameters of lenacapavir after an oral dose at 350 mg
fixed of Compound 1 in pentagastrin pretreated dogs (n = 3)

| | Animal ID | Dog 1 | Dog 2 | Dog 3 | Mean | SD |
|---|---|---|---|---|---|---|
| Leg 1 | AUCₗₐₛₜ(nM · h) | 110000 | 56000 | 64900 | 77100 | 29100 |
| (Compound | AUC_{inf} (nM · h) | 111000 | 56100 | 65000 | 77300 | 29400 |
| 1) | Cₘₐₓ (nM) | 5260 | 5330 | 5340 | 5310 | 44 |
| | Tₘₐₓ (h) | 2.00 | 2.00 | 2.00 | 2.00 | 0.00 |
| | % F | 25.9% | 12.2% | 13.2% | 17.1% | 7.7% |
| Leg 2 | AUCₗₐₛₜ(nM · h) | 303000 | 148000 | 147000 | 200000 | 89700 |
| (Compound | AUC_{inf} (nM · h) | 307000 | 148000 | 147000 | 201000 | 91700 |
| 1 + | Cₘₐₓ (nM) | 16300 | 10500 | 10900 | 12600 | 3240 |
| solution | Tₘₐₓ (h) | 2.00 | 2.00 | 2.00 | 2.00 | 0.00 |
| vehicle) | % F | 71.5% | 31.3% | 31.4% | 44.7% | 23.2% |

Plasma concentration-time profiles for lenacapavir in individual animals are shown in Tables 7-8. BLQ: Below the limit of quantification<1.0 nM.

TABLE 7
Individual lenacapavir plasma concentration (nM)
in male beagle dogs following an oral dose of
Compound 1 at 350 mg fixed dose (Leg 1)

| Time (h) | Dog 1 | Dog 2 | Dog 3 | Mean | SD |
|---|---|---|---|---|---|
| 0.00 | BLQ | BLQ | BLQ | NA | NA |
| 0.25 | 2.56 | 2.75 | 2.67 | 2.66 | 0.0954 |
| 0.50 | 37.3 | 26.7 | 11.2 | 25.1 | 13.1 |
| 1.00 | 1320 | 1190 | 1310 | 1273 | 72.3 |
| 2.00 | 5260 | 5330 | 5340 | 5310 | 44 |
| 4.00 | 3970 | 4100 | 4010 | 4027 | 67 |
| 6.00 | 2770 | 3170 | 3010 | 2983 | 201 |
| 8.00 | 1880 | 2240 | 2640 | 2253 | 380 |
| 12.0 | 1360 | 1290 | 1650 | 1433 | 191 |
| 24.0 | 1890 | 604 | 804 | 1099 | 692 |
| 48.0 | NS | 145 | 197 | 171 | 36.8 |
| 72.0 | 272 | 33 | 43 | 116 | 135 |
| 96.0 | 107 | 9.67 | 12.8 | 43.2 | 55.3 |
| 120 | 62.6 | 3.78 | 5.13 | 23.8 | 33.6 |
| 144 | 26.3 | 2.81 | 2.76 | 10.6 | 13.6 |
| 168 | 14.8 | BLQ | 1.86 | 8.33 | 9.15 |

TABLE 8
Individual lenacapavir plasma concentration (nM) in
male beagle dogs following an oral dose of Compound
1 at 350 mg fixed dose with solution vehicle (Leg 2)

| Time (h) | Dog 1 | Dog 2 | Dog 3 | Mean | SD |
|---|---|---|---|---|---|
| 0.00 | BLQ | BLQ | BLQ | NA | NA |
| 0.25 | 3.3 | 21 | 4.63 | 9.64 | 9.86 |
| 0.50 | 21.6 | 326 | 82 | 143 | 161 |
| 1.00 | 5710 | 2960 | 4790 | 4487 | 1400 |
| 2.00 | 16300 | 10500 | 10900 | 12567 | 3239 |
| 4.00 | 12700 | 9150 | 9550 | 10467 | 1944 |
| 6.00 | 7850 | 6230 | 5950 | 6677 | 1026 |
| 8.00 | 5900 | 4730 | 4340 | 4990 | 812 |
| 12.0 | 5450 | 3080 | 2800 | 3777 | 1456 |
| 24.0 | 4470 | 2400 | 2260 | 3043 | 1238 |
| 48.0 | 1940 | 534 | 639 | 1038 | 783 |
| 72.0 | 1010 | 144 | 148 | 434 | 499 |
| 96.0 | 479 | 37.8 | 40.7 | 186 | 254 |
| 120 | 244 | 8.32 | 12.8 | 88.4 | 135 |
| 144 | 106 | 5.06 | 2.89 | 38.0 | 58.9 |
| 168 | 85.2 | 2.16 | 2.52 | 30.0 | 47.8 |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A tablet comprising a compound of Formula Ia: 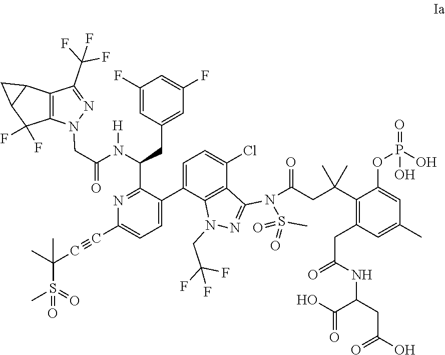 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]
or a pharmaceutically acceptable salt thereof, and one or more pharmaceutically acceptable excipients.

2. The tablet of claim 1, wherein the tablet comprises about 30 w/w % to about 80 w/w % of the compound of Formula Ia, or a pharmaceutically acceptable salt thereof.

3. The tablet of claim 1, wherein the tablet comprises about 55 w/w % to about 65 w/w % of the compound of Formula Ia, or a pharmaceutically acceptable salt thereof.

4. The tablet of claim 1, wherein the tablet comprises about 60 w/w % of the compound of Formula Ia, or a pharmaceutically acceptable salt thereof.

5. The tablet of claim 1, wherein the tablet comprises about 20 w/w % to about 55 w/w % of one or more fillers, about 1 w/w % to 15 w/w % of one or more disintegrants, and about 1 w/w % to 5 w/w % of one or more lubricants.

6. The tablet of claim 1, wherein:
the one or more fillers is selected from microcrystalline cellulose, mannitol, lactose monohydrate, or silicified microcrystalline cellulose;
the one or more disintegrants is crospovidone; and
the one or more lubricants is magnesium stearate.

7. The tablet of claim 1, wherein the tablet comprises about 20 w/w % to about 55 w/w % of microcrystalline cellulose.

8-9. (canceled)

10. The tablet of claim 1, wherein the tablet comprises about 5 w/w % to about 15 w/w % of crospovidone.

11-12. (canceled)

13. The tablet of claim 1, wherein the tablet comprises about 1 w/w % to about 5 w/w % of magnesium stearate.

14-15. (canceled)

16. The tablet of claim 1, wherein the tablet comprises:
about 30 w/w % to about 80 w/w % of the compound of Formula Ia, or a pharmaceutically acceptable salt thereof;
about 10 w/w % to about 35 w/w % of microcrystalline cellulose;
about 5 w/w % to about 15 w/w % of crospovidone; and
about 1 w/w % to about 5 w/w % of magnesium stearate.

17. (canceled)

18. The tablet of claim 1, wherein the tablet comprises:
about 60 w/w % of the compound of Formula Ia, or a pharmaceutically acceptable salt thereof;
about 28 w/w % of microcrystalline cellulose;
about 10 w/w % of crospovidone; and
about 2 w/w % of magnesium stearate.

19. (canceled)

20. The tablet of claim 1, wherein the tablet further comprises about 10 w/w % to about 30 w/w % of mannitol.

21. (canceled)

22. The tablet of claim 1, wherein the tablet further comprises about 0.1 w/w % to about 5 w/w % of poloxamer 407.

23. The tablet of claim 1, wherein the tablet comprises:
about 30 w/w % to about 80 w/w % of the compound of Formula Ia, or a pharmaceutically acceptable salt thereof;
about 0 w/w % to about 30 w/w % of mannitol;
about 10 w/w % to about 35 w/w % of microcrystalline cellulose;
about 1 w/w % to about 15 w/w % of crospovidone;
about 0 w/w % to about 5 w/w % poloxamer 407; and
about 1 w/w % to about 5 w/w % magnesium stearate.

24. The tablet of claim 1, wherein the tablet comprises about 1 mg to about 2000 mg of the compound of Formula Ia, or a pharmaceutically acceptable salt thereof.

25-32. (canceled)

33. The tablet of claim 1, wherein the tablet comprises about 900 mg of the compound of Formula Ia, or a pharmaceutically acceptable salt thereof.

34. The tablet of claim 1, wherein the tablet further comprises an outer film coat.

35-36. (canceled)

37. The tablet of claim 1, wherein the compound of Formula Ia, or a pharmaceutically acceptable salt thereof, is a compound of Formula Ib: 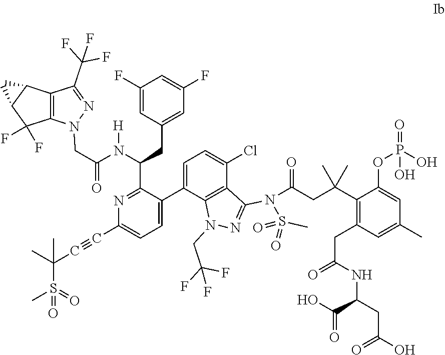 [SMILES: Cc1cc(CC(=O)N[C@@H](CC(=O)O)C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]
or a pharmaceutically acceptable salt thereof.

38-39. (canceled)

40. A method of preventing human immunodeficiency virus (HIV) infection in a patient, comprising administering to the patient a tablet of claim 1.

41. The method of claim 40, wherein the method comprises orally administering the tablet to the patient.

42. The method of claim 40, wherein the method comprises orally administering the tablet to the patient, once every month.

43-59. (canceled)

60. The method of claim 40, wherein the method comprises pre-exposure prophylaxis (PrEP).

61-63. (canceled)

64. The method of claim 40, wherein the tablet is administered before exposure of the patient to the HIV.

65-71. (canceled)

72. The method of claim 40, further comprising administering to the patient a PGP inhibitor.

73. The method of claim 72, wherein the PGP inhibitor is encequidar, or a pharmaceutically acceptable salt thereof.

74. The method of claim 72, wherein the PGP inhibitor is encequidar mesylate.

75. The method of claim 40, wherein the HIV is HIV-1.

76-90. (canceled)

91. The tablet of claim 1, wherein the tablet comprises about 450 mg of the compound of Formula Ia, or a pharmaceutically acceptable salt thereof.

92. The tablet of claim 1, wherein the tablet comprises about 600 mg of the compound of Formula Ia, or a pharmaceutically acceptable salt thereof.
